(19) United States
(12) Patent Application Publication
(10) Pub. No.: US 2007/0293451 A1
(43) Pub. Date: Dec. 20, 2007

(54) Regulation ao ApoB by Hsp110 proteins and related compositions and methods

(75) Inventors: Jeffrey L. Brodsky, Pittsburgh, PA (US); Edward A. Fisher, Scarsdale, NY (US)

Correspondence Address: JESSE A. HIRSHMAN, ESQ., 1722 MURRAY AVENUE, THIRD FLOOR, PITTSBURGH, PA, 15217, US

(73) Assignee: University of Pittsburgh, Pittsburgh, PA (US)

(21) Appl. No.: 11/799,697

(22) Filed: May 2, 2007

Related U.S. Application Data
(60) Provisional application No. 60/796,654, filed on May 2, 2006.

(51) Int. Cl. A61K 38/00; A61P 43/00; C07H 21/04; C12N 1/00; C12N 5/00; C12Q 1/68; G01N 33/53

(52) U.S. Cl. 514/44; 435/255.1; 435/325; 435/6; 435/7.1; 435/7.9; 536/23.5; 536/23.72; 536/23.74

(57) ABSTRACT

Hsp110 proteins are shown herein to stabilize nascent ApoB protein. Methods of down-regulating expression of ApoB, and thus down-regulating serum cholesterol levels, are therefore, provided, including, without limitation, inhibition of Hsp110 expression by any method, for example and without limitation, by RNA interference (RNAi). It also has been found that ApoB gene-containing cells are useful in identifying compounds and genes that affect the expression of longer forms of ApoB. Lastly, a firefly luciferase-derived polypeptide is shown herein to bind the peptide binding site of Hsp110, thus serving as a powerful analytical reagent for identifying compounds, such as small molecule compounds, that would interfere with Hsp110-moderated stabilization of ApoB.

### CROSS REFERENCE TO RELATED APPLICATIONS

This application claims priority to U.S. Provisional Patent Application No. 60/796,654, filed on May 2, 2006, and which is incorporated herein by reference in its entirety.

### STATEMENT REGARDING FEDERAL FUNDING

This invention was made with government support under Grant No. NIH R01 HL-058541 awarded by the National Institutes of Health. The government has certain rights in the invention.

Apolipoprotein B (ApoB, OMIM Reference No. 107730) is a very large (540 kDa) protein and is essential for the assembly and secretion of hepatic lipoproteins. ApoB is a major component of low density lipoprotein (LDL, the atherosclerosis-causing particle that transports most cholesterol in the blood), chylomicrons and very low density lipoprotein (VLDL). ApoB occurs in the plasma in 2 main forms, ApoB48 and ApoB100. In humans, the first is synthesized exclusively in the gut (specifically by intestinal cells), the second by the liver. ApoB100 is a component of VLDL, intermediate density lipoprotein (IDL) and low density lipoproteins (LDL) and contributes to hepatic and peripheral tissue uptake of LDL by receptor recognition. ApoB48 is an important component of chylomicrons (CM) and is required for their formation. ApoB48 lacks the part of ApoB100 that is recognized by the LDL receptor, which is probably why chylomicron remnants are predominantly cleared by the liver via its ApoE content and LDL provides cholesterol to peripheral tissues. A low plasma level of ApoB is indicative of hypobetalipoproteinemia, while a high plasma level is a major risk factor for the development of atherosclerosis. During the translation of ApoB 100 in the liver, lipids are transferred to the nascent protein by the ER-resident microsomal triglyceride transfer protein (MTP) to form a “primordial lipoprotein.” In hepatocytes and hepatocarcinoma-derived cell lines, ApoB100 either undergoes assembly with lipids and secretion or is subjected to endoplasmic reticulum (ER) retention and intracellular degradation. The degree of proteasomal degradation appeared to be regulated by the availability of the lipid ligands for ApoB 100 within the ER. ApoB 100 also undergoes degradation according to a non-ubiquitin-dependent pathway. In species such as rodents in which apoB48 is also made in the liver, apoB48 is translated, assembled into lipoproteins and degraded in a similar manner, as are shorter forms ApoB (e.g., ApoB29, see below).

ER degradation has been shown to be carried out by the ubiquitin-proteasome pathway and involves cytosolic Hsp70 among other factors. There are a number of ER luminal (soluble) and ER transmembrane proteins that have also been reported to undergo proteasomal degradation. This process has been referred to as ER-associated degradation (ERAD) and for a number of substrate proteins a common scenario appears to be that the nascent protein undergoes translocation, followed by retrotranslocation and release into the cytosol, where it is degraded by the proteasome. The mechanism of ApoB degradation is similar, but not identical to this model. ApoB can assume a bitopic topology during or shortly after translocation, resulting in domains that can be accessed by cytosolic or ER luminal factors. When the ApoB lipid ligand is not present in sufficient amounts within the ER and/or MTP cannot deliver the lipid to ApoB, a cytoplasmic loop is formed. The cytoplasmic loop first attracts molecular chaperones, including Hsp70 and Hsp90. The chaperone-bound cytoplasmic loop then recruits components of the ubiquitination machinery to ubiquitinate the cytoplasmic loop, thereby rendering ApoB subject to proteasome degradation. Much of this process has been worked out in in vitro systems (Gusarova, V. et al. (2001), “Apolipoprotein B Degradation is Promoted by the Molecular Chaperones Hsp90 and Hsp70,” J. Biol Chem. 276(27):24891-24900).

Molecular chaperones are required to catalyze a number of cellular activities, including protein folding, transport, and degradation. These activities can be highly interrelated. For example, chaperones that fail to refold denatured proteins may redirect the polypeptide to proteolytic machines in the cell. In addition, chaperones can prevent premature folding or aggregation to deliver nascent polypeptides to translocation complexes localized at the ER or mitochondrial membranes. Finally, specific molecular chaperones may deliver protein substrates to other chaperones. Notably, chaperone “bucket-brigades” were observed when the protein-folding activities of the DnaK-DnaJ-GrpE and GroEL/GroES complexes were first uncovered, and during the translocation and subsequent folding of proteins in the mitochondria. In another example, the mammalian heat-shock protein 110 (Hsp110) molecular chaperone acts as a “holdase,” binding to unfolded proteins and preventing their aggregation until ATP-dependent refolding is catalyzed by the Hsp70 and Hsp40 chaperones (Oh, H. J., et al,. J. Biol Chem. 1997 Dec 12;272(50):31636-40). Not surprisingly, the expression of mammalian Hsp110 is induced by heat, and overexpression of Hsp110 confers thermotolerance to Rat-1 and HeLa cells. Subsequent studies determined that the holdase activity of mammalian Hsp110 requires its putative peptide-binding and C-terminal domains, but not the ATP-binding domain (Oh, H. J., et al., J. Biol Chem. 1999 May 28;274(22):15712-8).

To date, the role of the Hsp110 family of molecular chaperones has been incompletely defined. Deletion of one of the genes encoding an Hsp110 chaperone in Saccharomyces cerevisiae (S. cerevisiae), known as SSE1, results in poor viability at every temperature examined (Mukai H, Kuno T, Tanaka H, Hirata D, Miyakawa T, Tanaka C. Isolation and characterization of SSE1 and SSE2, new members of the yeast HSP70 multigene family. Gene. 1993 Sep 30;132(1):57-66.; Shirayama M, Kawakami K, Matsui Y, Tanaka K, Toh-e A. MSI3, a multicopy suppressor of mutants hyperactivated in the RAS-cAMP pathway, encodes a novel HSP70 protein of Saccharomyces cerevisiae. Mol Gen Genet. 1993 Sep;240(3):323-32). Although it is not clear how Sse1p supports optimal cell growth, purified yeast Sse1p, like mammalian Hsp110, exhibits holdase activity (Brodsky J L, Wemer E D, Dubas M E, Goeckeler J L, Kruse K B, McCracken A A). The requirement for molecular chaperones during endoplasmic reticulum-associated protein degradation demonstrates that protein export and import are mechanistically distinct. J. Biol Chem. 1999 Feb 5;274(6):3453-60.). In addition, Sselp interacts biochemically and genetically with the yeast Hsp90 complex (Liu X D, Morano K A, Thiele D J). The yeast Hsp110 family member, Sse1, is an Hsp90 cochaperone. J. Biol Chem. 1999 Sep 17;274(38):26654-60.), and yeast lacking the Hsp90 homologs Hsc82 (constitutively expressed at high levels and moderately stress inducible) and Hsp82 (constitutively expressed at low levels but heat inducible) are inviable (Borkovich K A, Farrelly F W, Finkelstein D B, Taulien J, Lindquist S. hsp82 is an essential protein that is required in higher concentrations for growth of cells at higher temperatures. Mol Cell Biol. 1989 Sep;9(9):3919-30; Nathan D F, Lindquist S. Mutational analysis of Hsp90 function: interactions with a steroid receptor and a protein kinase. Mol Cell Biol. 1995 Jul;15(7):3917-25).

Goeckler et al., (2002) Mol. Biol. Cell. 13:2760-70, offered some insight into the role of yeast Hsp110 homolog Sselp in yeast, using both genetic and biochemical methods. First, they found that overexpression of Sse1p rescues the thermosensitivity of yeast containing a temperature-sensitive mutation (“ydj1-151”) in the gene encoding a major Hsp40, YDJ1. Purification and functional analyses of wild-type and mutant forms of Sse1p indicated that the presumptive ATP-binding domain is dispensable for the in vitro holdase activity of the chaperone. However, yeast overexpressing holdase-proficient Sselp mutants could not rescue the thermosensitive ydj1-151 growth defect. Those results indicate that the contribution of Sse1p to cell growth arises at least in part from facilitating Hsp90-dependent functions, and more generally that demonstrations of chaperone holdase activity might not correspond to an essential in vivo activity. Despite this increased insight into the role of Hsp110, much about this protein remains unknown.

### SUMMARY

Surprisingly, as shown in further detail below, Hsp110 family members have the opposite effect on ApoB degradation as compared to Hsp70 and Hsp90. In yeast lysate systems, less ApoB degradation was seen in the presence of wild-type (wt.) Hsp110, as compared to assays in which Hsp110 is absent. Thus, Hsp110 is considered to be a stabilizer of ApoB. Hsp110 has not previously been shown to be involved in ApoB degradation pathways, and this discovery has made Hsp110 a suitable target for drug discovery because lowering of plasma ApoB will result in lower levels of LDLs. In another aspect of the present invention, yeast cells expressing a shortened version of ApoB, ApoB29, handle ApoB identically to the manner in which longer forms of ApoB are treated in in vitro systems and in mammalian cells; i.e. the requirements for the degradation are identical between each system. Therefore, the engineered yeast strain or mammalian cells expressing this shorter form of the protein may be co-opted to screen for compounds that affect ApoB expression and for genes, that when mutated, affect ApoB stability. It also has been found that a portion of the firefly luciferase protein binds the protein binding site of the yeast Hsp110 protein, Sse1p, and these tools can therefore be employed as an analytical tool to identify compounds that interfere with Hsp110's stabilizing effect on ApoB.

In one embodiment, a method of identifying compounds that affect ApoB degradation is provided. The method comprises contacting one or more cell populations in one or more discrete physical locations, in which the cells of the cell populations comprise an ApoB29 gene comprising an ApoB29 sequence, with one or more compounds and determining if the compound or compounds affect expression of the ApoB29 gene. The cell may be mammalian or yeast. In one embodiment, the ApoB29 gene encodes a protein comprising approximately the N-terminal 1374 amino acids of ApoB100, and in another, approximately amino acids 27-1374 of ApoB100. In a yeast cell, the ApoB29 gene may encode a yeast prepro sequence consisting of approximately amino acids 1-100 of yeast mating factor alpha 1 locus, which in one embodiment, replaces the N-terminal 26 amino acids of ApoB29. In one embodiment, the ApoB29 gene is inducible, for example and without limitation, when in a yeast cell, by galactose-induction, using, for example, a GAL 1, 10 promoter. Two or more compounds may be screened in parallel in an array of cell populations, such as in a 96-well dish.

In another embodiment, a method is provided of treating high serum cholesterol in a patient in which cholesterol levels are raised, comprising, down-regulating expression of an Hsp110 protein, thereby decreasing ApoB protein levels in the patient. In one preferred embodiment, the patient is a human patient. In one embodiment, the method comprises administering to the patient an siRNA that corresponds to an RNAi target in an Hsp110 and which down-regulates expression of the Hsp110, such as, without limitation, the RNAi targets or the siRNAs provided herein.

In another embodiment, a cell is provided comprising an ApoB29 gene comprising a sequence encoding an ApoB29 protein, which is useful in the methods described herein.

In yet another embodiment, a method is provided of determining if a compound or composition binds an Hsp110 protein. The method comprises determining if the compound or composition dissociates or interferes with association of a complex of the Hsp 110 protein or a portion thereof containing a protein-binding region of the Hsp110, and an Hsp 110-binding polypeptide comprising the amino acid sequence LICGFRVVLMYRF (SEQ ID NO: 1), which may be less than about 100, 50, 25 or 15 amino acids. In one non-limiting embodiment, the polypeptide consists of the amino acid sequence LICGFRVVLMYRF (SEQ ID NO: 1), and is labeled with a fluorescein compound, such as, without limitation, 6-carboxyfluorescein-aminohexanoic acid.

In another embodiment, provided is an isolated nucleic acid comprising a selectable marker useful in yeast, an ApoB29 gene sequence encoding a protein comprising, from its N-terminal to its C-terminal, a yeast prepro sequence, an ApoB29 sequence consisting of approximately amino acids 27-1374 of ApoB100 and one or more binding partners.

In another embodiment, a method is provided of identifying genes affecting ApoB steady-state levels, comprising determining the affect of a mutation in a gene in a cell on expression of an ApoB gene comprising a sequence encoding an ApoB protein.

### BRIEF DESCRIPTION OF THE DRAWINGS

FIGS. 1A and 1B show ApoB degradation in vitro using wild type (SSE1) and Hsp110 mutant (sse1, Δ) cytosol. Degradation was also assessed in the presence or absence of MG 132, a specific inhibitor of the proteasome. In FIG. 1A, the “%ApoB remaining” refers to the relative amount of protein in the absence of MG132 divided by the amount in the presence of MG132 at each time point, thus reflecting only the degree of proteasome-mediated degradation.

FIGS. 2A and 2B show ApoB degradation in vitro using wild type (SSB1SSB2) and mutant (sse1ssb2) cytosol.

FIG. 3 is a graph showing that active, recombinant Sse1p, which was added into the assay as indicated, stabilizes ApoB in vitro.

FIG. 4A is a map of plasmid pSLW1-B29 (also referred to as “pJJB20-B29HA”).

FIGS. 4B and 4C are western blots of yeast extracts using anti-HA (FIG. 4B) or anti-ApoB (FIG. 4C) antisera, as described below.

FIG. 5 shows western blots indicating membrane association of ApoB29 in yeast, as described below.

FIGS. 6A and 6B show slowing of ApoB29 degradation in cim3-1 mutant cells as compared to isogenic wild type yeast (CIM3). In yeast, proof-of-point that the proteasome is required to degrade ApoB and that the in vivo requirements mimic those observed in vitro and in mammalian cells.

FIGS. 7A and 7B show that Sselp stabilizes ApoB in vivo.

FIGS. 8A and 8B show ApoB29 degradation measured in wild type cells (SSB wt.) and in yeast lacking the Ssb chaperones (ssb1Δssb2Δ).

FIGS. 9A and 9B show the effect of Hsp110 over-expression on ApoB100 secretion from McArdle cells, a standard rat hepatoma cell model of lipoprotein metabolism. ApoB100 (FIG. 9A) and albumin (FIG. 9B) biogenesis and secretion were assessed by published pulse-chase analytical methods.

FIG. 10 is a graph showing full length and the peptide binding domain of Sse1p binding to peptide 6-carboxyfluorescein-aminohexanoic acid-linked-LICGFRVVLMYRF (SEQ ID NO: 1) (which derives from firefly luciferase), assessed by fluorescence anisotropy.

FIG. 11 provides the amino acid sequence of the human ApoB precursor, ApoB100 (GenBank Accession No. NP_{—}000375) (SEQ ID NO: 1).

FIG. 12 provides the amino acid sequence of Sse1p (GenBank Accession No. NP_{—}015219) (SEQ ID NO: 2).

FIG. 13 provides the amino acid sequence for one of the homologous human Hsp 110 proteins, Heat-shock protein Apg-1 (HSPA4L, GenBank Accession No. NP_{—}055093. SEQ ID NO: 3).

FIG. 14A provides the nucleotide sequence for a human Hsp110 cDNA (FIG. 14A, SEQ ID NO: 4, Homo sapiens heat shock 70 kDa protein 4-like,GenBank Accession No. NM_{—}014278).

FIG. 14B shows the reverse complement RNA sequence of the sequence shown in FIG. 14A, SEQ ID NO: 1).

### DETAILED DESCRIPTION

As stated above, Hsp110 surprisingly has the opposite effect on ApoB degradation as compared to Hsp70 and Hsp90. In yeast lysate systems, less ApoB degradation was seen in the presence of wild-type (wt.) Hsp110 (in yeast, Sse1p), as compared to assays in which Hsp110 is absent. Thus, Hsp110 is considered to be a stabilizer of ApoB. Consistent with this hypothesis supplementing cytosols with Sse1p stabilizes ApoB48 (as an experimental substitute for ApoB100), and yeast lacking SSE1 degrade ApoB29 (as an experimental substitute for ApoB100) faster. Hsp110 has not previously been shown to be involved in ApoB degradation pathways, and this discovery has made Hsp110 a suitable target for drug discovery because lowering of plasma ApoB will result in lower levels of LDLs.

As used herein “ApoB,” refers generally to ApoB100, preferably human ApoB100, though as a class it includes ApoB48 and ApoB29, which, although naturally-occurring variants of human ApoB100, are also experimental models for ApoB100, as described herein, and, to some extent, by others. Nevertheless, ApoB100, and in one embodiment, human ApoB100, is the ultimate clinically-relevant therapeutic target due to its role in LDL formation.

For this reason, provided herein is a method of decreasing ApoB levels in a patient, typically a human patient, comprising inhibiting expression and/or function of an Hsp110 by any effective method. Because Hsp110 is not considered essential (Shirayama M, et al. Mol Gen Genet. 1993. 240(3):323-32 and Mukai H, et al. Gene. 1993. 132(1):57-66), down-regulation of Hsp110 expression is a realistic method for controlling circulating ApoB, and thus cholesterol, for example, and without limitation, in humans. Targeting of heat shock proteins is a viable therapeutic method. For example in the context of cancer therapies, geldanamycin and its analogs, such as 17-dimethylaminoethylamino- 17-demethoxygeldenamycin (17-DMAG) or 17-(allylamino)-17-demethoxy-geldanamycin (17-AAG) are being investigated for many therapeutic uses, for example and without limitation as shown in U.S. Pat. No. 6,890,917. Any method of inhibiting expression and/or function of Hsp110 in a patient may be employed to achieve the therapeutic goal of lowering ApoB levels, including, without limitation, RNA interference (RNAi, for example and without limitation as described below), antisense RNA, small molecule inhibitors of Hsp110 function, activity and/or binding or binding reagents that interfere with Hsp110 function in ApoB expression.

In one method of modulating ApoB expression in a patient, RNA interference (RNAi) is a relatively new method for gene silencing that has seen unprecedented success, even in vivo. (see, e.g., Fire A, et al. (1998) Nature. 391(6669) 806-11; Tuschl T, et al. (1999) Genes Dev 13(24) 3191-7; Zamore P D, et al. (2000) Cell 101(1) 25-33; Martinez J, et al. (2002) Cell 110(5) 563-74; Nykanen A, et al. Haley B, et al. (2001) Cell 107(3) 309-21; Elbashir S M, et al. (2001) EMBO J. 20(23) 6877-88; Schwarz D, et al. (2003) Cell 115(2) 199-208; Khvorova A, et al. (2003) Cell 115(2) 209-16; Jackson aL, et al. (2003) Nat Biotechnol. 21(6) 635-7 and Persengiev S P, et al. (2004) RNA 10(1), 12-18). U.S. patent application Ser. No. 20050287558, incorporated herein by reference for its technical disclosure, provides antisense and siRNA compounds, compositions and methods for modulating the expression of apolipoprotein B in vivo. Thus, regulation of ApoB directly by antisense and/or RNAi has been achieved in non-human primates (for RNAi, see, Zimmerman, T. S., et al. (2006), DOI:10.1038/nature04688), and in mice (Crooke R M, Graham M J, Lemonidis K M, Whipple C P, Koo S, Perera R J. An apolipoprotein B antisense oligonucleotide lowers LDL cholesterol in hyperlipidemic mice without causing hepatic steatosis J Lipid Res. 2005 May;46(5):872-84. Epub 2005 Feb 16), and given that RNAi technology is so robust, even in vivo, it follows that down-regulation of Hsp110 by RNAi would have an effect on ApoB levels. As used herein, an effector compound or composition, such as siRNA, is said to “modulate” expression of a gene product or levels of a compound or composition, in cells, tissue, organs and/or systemically and/or in bodily fluids, including, without limitation, blood, serum, plasma, cerebrospinal fluid, and/or lymph, if that effector causes expression of the gene product to change and/or affects levels of that product systemically and/or in the cell, tissue, organ and/or bodily fluid.

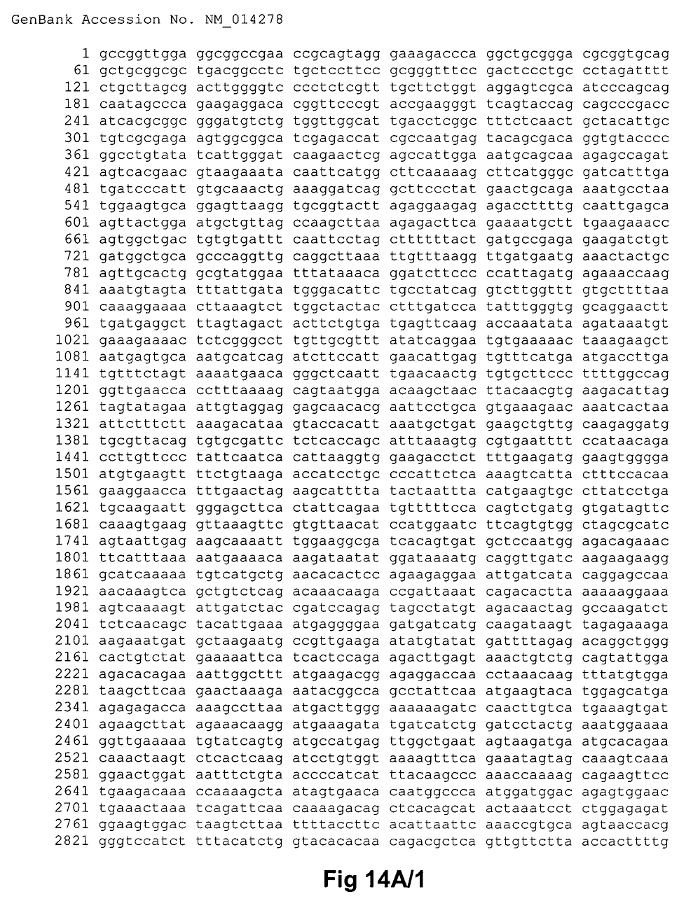

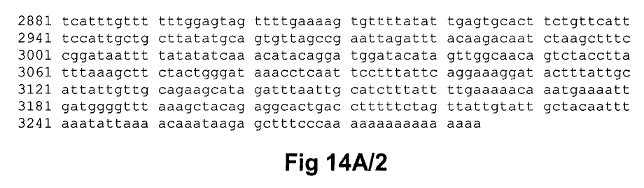

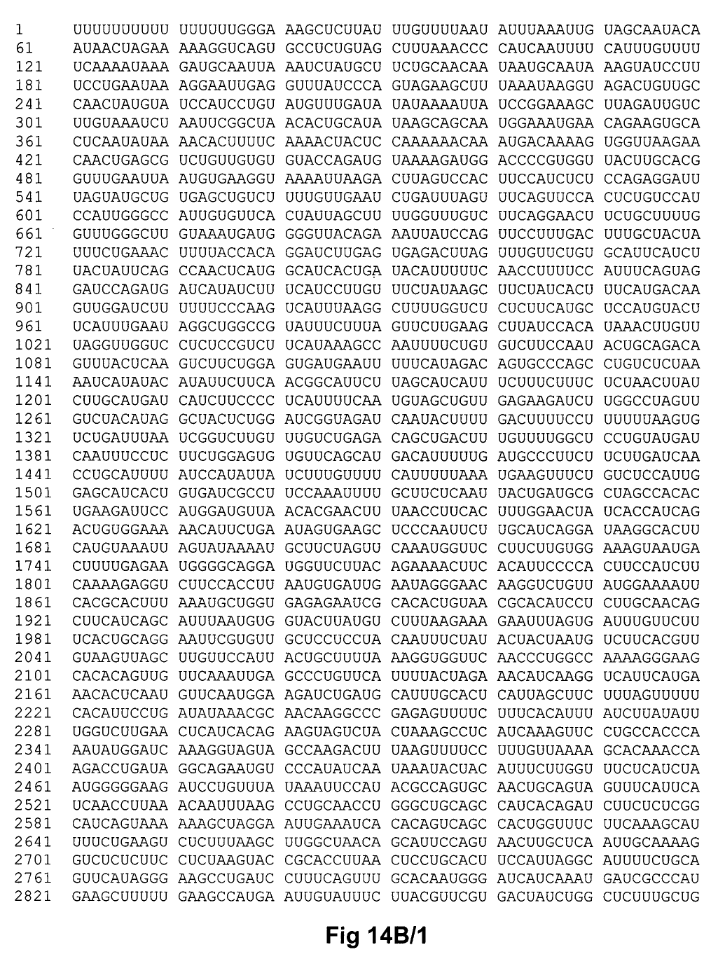

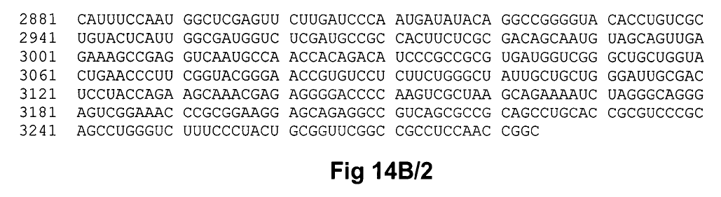

Ambion (www.ambion.com/techlib/misc/siRNA_tools.html) provides a tutorial/program to identify siRNAs for any given gene. About 50% of siRNAs designed using this tool purportedly will reduce target gene expression by >50%. The program utilizes the protocols “described by Tuschl and colleagues.” Note that the identified sequences below are 21 nucleotides. Optimized siRNAs contain much lower GC content—besides the standard AA overhangs—and selecting for those with 30% GC content somewhat reduces the total number of potential siRNAs identified. To further optimize the target selectivity, one can perform BLAST, or other searches on public databases, such as, without limitation, GenBank. In any event, for human Hsp110 (Apg-1, GenBank Accession No. NM_{—}014278, FIG. 14A, see, also, the protein sequence provided in FIG. 13, GenBank Accession No. NP_{—}055093, see, Nonoguchi,K., et al. “Cloning of human cDNAs for Apg-1 and Apg-2, members of the Hsp110 family, and chromosomal assignment of their genes” Gene 237 (1), 21-28 (1999)), the following 279 siRNAs were obtained. This strategy can also be performed on the other human Hsp110 homologues, including, without limitation, HSPH1 (GenBank Accession Nos. NM_{—}006644 and Q92598), human heat shock heat shock 105 kD protein (GenBank Accession No. NP_{—}006635) and Apg-2 (GenBank Accession No. AB023420), and potential siRNAs can be identified using the identical method. Regardless of the Hsp110 target, the number of potential siRNAs can be further refined using other tools commercially available from Ambion and others, for example and without limitation, by reducing GC content and/or by performing a BLAST search or other searches. The following are non-limiting examples of suitable siRNA targets for a human Hsp110 (Apg-1, GenBank Accession No. NM_{—}014278):

target sequence 1: AACCGCAGTAGGGAAAGACCC (residues 19-39 of SEQ ID NO: 5), position in gene sequence: 20, GC content: 57.1%, sense strand siRNA: CCGCAGUAGGGAAAGACCCUU (SEQ ID NO: 2), antisense strand siRNA: GGGUCUUUCCCUACUGCGGUU (residues 3246-3266 of SEQ ID NO: 6);

target sequence 2: AAAGACCCAGGCTGCGGGACG (residues 32-52 of SEQ ID NO: 5), position in gene sequence: 33, GC content: 66.7%, sense strand siRNA: AGACCCAGGCUGCGGGACGUU (SEQ ID NO: 3), antisense strand siRNA: CGUCCCGCAGCCUGGGUCUUU (residues 3233-3253 of SEQ ID NO: 6);

target sequence 3: AATCCCAGCAGCAATAGC (residues 170-187 of SEQ ID NO: 5), position in gene sequence: 176, GC content: 42.9%, sense strand siRNA: UCCCAGCAGCAAUAGCUU (SEQ ID NO: 4), antisense strand siRNA: GCUAUUGCUGCUGGGAWU (residues 3098-3113 of SEQ ID NO: 6);

target sequence 4: AATAGCCCAGAAGAGGACACG (residues 182-202 of SEQ ID NO: 5), position in gene sequence: 191, GC content: 52.4%, sense strand siRNA: UAGCCCAGAAGAGGACACGUU (SEQ ID NO: 5), antisense strand siRNA: CGUGUCCUCUUCUGGGCUAUU (residues 3083-3103 of SEQ ID NO: 6);

target sequence 5: AAGAGGACACGGTTCCCGTAC (residues 192-212 of SEQ ID NO: 5), position in gene sequence: 201, GC content: 57.1%, sense strand siRNA: GAGGACACGGUUCCCGUACUU (SEQ ID NO: 6), antisense strand siRNA: GUACGGGAACCGUGUCCUCUU (residues 3073-3093 of SEQ ID NO: 6);

target sequence 6: AAGGGTTCAGTACCAGCAGCC (residues 215-235 of SEQ ID NO: 5), position in gene sequence: 224, GC content: 57.1%, sense strand siRNA: GGGUUCAGUACCAGCAGCCUU (SEQ ID NO: 7), antisense strand siRNA: GGCUGCUGGUACUGAACCCUU (residues 3050-3070 of SEQ ID NO: 6);

target sequence 7: AACTGCTACATTGCTGTC (residues 287-304 of SEQ ID NO: 5), position in gene sequence: 299, GC content: 38.1%, sense strand siRNA: CUGCUACAUUGCUGUCUU (SEQ ID NO: 8), antisense strand siRNA: GACAGCAAUGUAGCAGUU (residues 2981-2998 of SEQ ID NO: 6);

target sequence 8: AAGTGGCGGCATCGAGACCAT (residues 310-330 of SEQ ID NO: 5), position in gene sequence: 325, GC content: 57.1%, sense strand siRNA: GUGGCGGCAUCGAGACCAUUU (SEQ ID NO: 9), antisense strand siRNA: AUGGUCUCGAUGCCGCCACUU (residues 2955-2975 of SEQ ID NO: 6);

target sequence 9: AATGAGTACAGCGACAGGTGT (residues 335-355 of SEQ ID NO: 5), position in gene sequence: 350, GC content: 47.6%, sense strand siRNA: UGAGUACAGCGACAGGUGUUU (SEQ ID NO: 10), antisense strand siRNA: ACACCUGUCGCUGUACUCAUU (residues 2930-2950 of SEQ ID NO: 6);

target sequence 10: AAGAACTCGAGCCATTGGAAA (residues 382-402 of SEQ ID NO: 5), position in gene sequence: 400, GC content: 42.9%, sense strand siRNA: GAACUCGAGCCAUUGGAAAUU (SEQ ID NO: 11), antisense strand siRNA: UUUCCAAUGGCUCGAGUUCUU (residues 2883-2903 of SEQ ID NO: 6);

target sequence 11: AACTCGAGCCATTGGAAATGC (residues 385-405 of SEQ ID NO: 5), position in gene sequence: 403, GC content: 47.6%, sense strand siRNA: CUCGAGCCAUUGGAAAUGCUU (SEQ ID NO: 12), antisense strand siRNA: GCAUUUCCAAUGGCUCGAGUU (residues 2880-2900 of SEQ ID NO: 6);

target sequence 12: AAATGCAGCAAAGAGCCAGAT (residues 400-420 of SEQ ID NO: 5), position in gene sequence: 418, GC content: 42.9%, sense strand siRNA: AUGCAGCAAAGAGCCAGAUUU (SEQ ID NO: 13), antisense strand siRNA: AUCUGGCUCUUUGCUGCAUUU (residues 2865-2885 of SEQ ID NO: 6);

target sequence 13: AAAGAGCCAGATAGTCAC (residues 409-426 of SEQ ID NO: 5), position in gene sequence: 427, GC content: 38.1%, sense strand siRNA: AGAGCCAGAUAGUCACUU (SEQ ID NO: 14), antisense strand siRNA: GUGACUAUCUGGCUCUUU (residues 2859-2876 of SEQ ID NO: 6);

target sequence 14: AACGTAAGAAATACAATTCAT (residues 428-448 of SEQ ID NO: 5), position in gene sequence: 449, GC content: 23.8%, sense strand siRNA: CGUAAGAAAUACAAUUCAUUU (SEQ ID NO: 15), antisense strand siRNA: AUGAAUUGUAUUUCUUACGUU (residues 2837-2857 of SEQ ID NO: 6);

target sequence 15: AAGAAATACAATTCATGGCTT (residues 433-453 of SEQ ID NO: 5), position in gene sequence: 454, GC content: 28.6%, sense strand siRNA: GAAAUACAAUUCAUGGCUUUU (SEQ ID NO: 16), antisense strand siRNA: AAGCCAUGAAUUGUAUUUCUU (residues 2832-2852 of SEQ ID NO: 6);

target sequence 16: AAATACAATTCATGGCTTCAA436-456 of SEQ ID NO: 5), position in gene sequence: 457, GC content: 28.6%, sense strand siRNA: AUACAAUUCAUGGCUUCAAUU (SEQ ID NO: 17), antisense strand siRNA: UUGAAGCCAUGAAUUGUAUUU2829-2849 of SEQ ID NO: 6);

target sequence 17: AATTCATGGCTTCAAAAAGCT (residues 442-462 of SEQ ID NO: 5), position in gene sequence: 463, GC content: 33.3%, sense strand siRNA: UUCAUGGCUUCAAAAAGCUUU (SEQ ID NO: 18), antisense strand siRNA: AGCUUUUUGAAGCCAUGAAUU (residues 2823-2843 of SEQ ID NO: 6);

target sequence 18: AAAAAGCTTCATGGGCGATCA (residues 455-475 of SEQ ID NO: 5), position in gene sequence: 476, GC content: 42.9%, sense strand siRNA: AAAGCUUCAUGGGCGAUCAUU (SEQ ID NO: 19), antisense strand siRNA: UGAUCGCCCAUGAAGCUUUUU (residues 2810-2830 of SEQ ID NO: 6);

target sequence 19: AAAGCTTCATGGGCGATCATT (residues 457-477 of SEQ ID NO: 5), position in gene sequence: 478, GC content: 42.9%, sense strand siRNA: AGCUUCAUGGGCGAUCAUUUU (SEQ ID NO: 20), antisense strand siRNA: AAUGAUCGCCCAUGAAGCUUU (residues 2808-2828 of SEQ ID NO: 6);

target sequence 20: AAACTGAAAGGATCAGGCTTC495-515 of SEQ ID NO: 5), position in gene sequence: 519, GC content: 42.9%, sense strand siRNA: ACUGAAAGGAUCAGGCUUCUU (SEQ ID NO: 21), antisense strand siRNA: GAAGCCUGAUCCUUUCAGUUU2770-2790;

target sequence 21: AAAGGATCAGGCTTCCCTATG (residues 501-521 of SEQ ID NO: 5), position in gene sequence: 525, GC content: 47.6%, sense strand siRNA: AGGAUCAGGCUUCCCUAUGUU (SEQ ID NO: 22), antisense strand siRNA: CAUAGGGAAGCCUGAUCCUUU (residues 2764-2784 of SEQ ID NO: 6);

target sequence 22: AACTGCAGAAAATGCCTAA (residues 522-540 of SEQ ID NO: 5), position in gene sequence: 546, GC content: 33.3%, sense strand siRNA: CUGCAGAAAAUGCCUAAUU (SEQ ID NO: 23), antisense strand siRNA: UUAGGCAUUUCUGCAGUU (residues 2745-2763 of SEQ ID NO: 6);

target sequence 23: AAAATGCCTAATGGAAGT (residues 530-547 of SEQ ID NO: 5), position in gene sequence: 554, GC content: 28.6%, sense strand siRNA: AAUGCCUAAUGGAAGUUU (SEQ ID NO: 24), antisense strand siRNA: ACUUCCAUUAGGCAUUUU (residues 2738-2755 of SEQ ID NO: 6);

target sequence 24: AATGCCTAATGGAAGTGC (residues 532-549 of SEQ ID NO: 5), position in gene sequence: 556, GC content: 38.1%, sense strand siRNA: UGCCUAAUGGAAGUGCUU (SEQ ID NO: 25), antisense strand siRNA: GCACUUCCAUUAGGCAUU (residues 2736-2753 of SEQ ID NO: 6);

target sequence 25: AATGGAAGTGCAGGAGTT (residues 539-556 of SEQ ID NO: 5), position in gene sequence: 563, GC content: 38.1%, sense strand siRNA: UGGAAGUGCAGGAGUUUU (SEQ ID NO: 26), antisense strand siRNA: AACUCCUGCACUUCCAUU (residues 2729-2746 of SEQ ID NO: 6);

target sequence 26: AAGTGCAGGAGTTAAGGTGCG (residues 544-564 of SEQ ID NO: 5), position in gene sequence: 571, GC content: 52.4%, sense strand siRNA: GUGCAGGAGUUAAGGUGCGUU (SEQ ID NO: 27), antisense strand siRNA: CGCACCUUAACUCCUGCACUU (residues 2721-2741 of SEQ ID NO: 6);

target sequence 27: AAGGTGCGGTACTTAGAGGAA (residues 557-577 of SEQ ID NO: 5), position in gene sequence: 584, GC content: 47.6%, sense strand siRNA: GGUGCGGUACUUAGAGGAAUU (SEQ ID NO: 28), antisense strand siRNA: UUCCUCUAAGUACCGCACCUU (residues 2708-2728 of SEQ ID NO: 6);

target sequence 28: AAGAGAGACCTTTTGCAATTG (residues 576-596 of SEQ ID NO: 5), position in gene sequence: 603, GC content: 38.1%, sense strand siRNA: GAGAGACCUUUUGCAAUUGUU (SEQID NO: 29), antisense strand siRNA: CAAUUGCAAAAGGUCUCUCUU (residues 2689-2709 of SEQ ID NO: 6);

target sequence 29: AATTGAGCAAGTTACTGG (residues 592-609 of SEQ ID NO: 5), position in gene sequence: 619, GC content: 33.3%, sense strand siRNA: UUGAGCAAGUUACUGGUU (SEQ ID NO: 30), antisense strand siRNA: CCAGUAACUUGCUCAAUU (residues 2676-2693 of SEQ ID NO: 6);

target sequence 30: AATGCTGTTAGCCAAGCTTAA (residues 610-630 of SEQ ID NO: 5), position in gene sequence: 640, GC content: 38.1%, sense strand siRNA: UGCUGUUAGCCAAGCUUAAUU (SEQ ID NO: 31), antisense strand siRNA: UUAAGCUUGGCUAACAGCAUU (residues 2655-2675 of SEQ ID NO: 6);

target sequence 31: AAGCTTAAAGAGACTTCAGAA (residues 623-643 of SEQ ID NO: 5), position in gene sequence: 653, GC content: 33.3%, sense strand siRNA: GCUUAAAGAGACUUCAGAAUU (SEQ ID NO: 32), antisense strand siRNA: UUCUGAAGUCUCUUUAAGCUU (residues 2642-2662 of SEQ ID NO: 6);

target sequence 32: AAAGAGACTTCAGAAAATGCT (residues 629-649 of SEQ ID NO: 5), position in gene sequence: 659, GC content: 33.3%, sense strand siRNA: AGAGACUUCAGAAAAUGCUUU (SEQ ID NO: 33), antisense strand siRNA: AGCAUUUUCUGAAGUCUCUUU (residues 2636-2656 of SEQ ID NO: 6);

target sequence 33: AAAATGCTTTGAAGAAACC (residues 642-660 of SEQ ID NO: 5), position in gene sequence: 672, GC content: 28.6%, sense strand siRNA: AAUGCUUUGAAGAAACCUU (SEQ ID NO: 34), antisense strand siRNA: GGUUUCUUCAAAGCAUUUU (residues 2625-2643 of SEQ ID NO: 6);

target sequence 34: AATGCTTTGAAGAAACCA (residues 644-661 of SEQ ID NO: 5), position in gene sequence: 674, GC content: 28.6%, sense strand siRNA: UGCUUUGAAGAAACCAUU (SEQ ID NO: 35), antisense strand siRNA: UGGUUUCUUCAAAGCAUU (residues 2624-2641 of SEQ ID NO: 6);

target sequence 35: AAGAAACCAGTGGCTGAC (residues 653-670 of SEQ ID NO: 5), position in gene sequence: 683, GC content: 42.9%, sense strand siRNA: GAAACCAGUGGCUGACUU (SEQ ID NO: 36), antisense strand siRNA: GUCAGCCACUGGUUUCUU (residues 2615-2632 of SEQ ID NO: 6);

target sequence 36: AAACCAGTGGCTGACTGT (residues 656-673 of SEQ ID NO: 5), position in gene sequence: 686, GC content: 42.9%, sense strand siRNA: ACCAGUGGCUGACUGUUU (SEQ ID NO: 37), antisense strand siRNA: ACAGUCAGCCACUGGUUU (residues 2612-2629 of SEQ ID NO: 6);

target sequence 37: AATTCCTAGCTTTTTTACTGA (residues 682-702 of SEQ ID NO: 5), position in gene sequence: 715, GC content: 28.6%, sense strand siRNA: UUCCUAGCUUUUUACUGAUU (SEQ ID NO: 38), antisense strand siRNA: UCAGUAAAAAAGCUAGGAAUU (residues 2583-2603 of SEQ ID NO: 6);

target sequence 38: AAGATCTGTGATGGCTGC (residues 712-729 of SEQ ID NO: 5), position in gene sequence: 745, GC content: 42.9%, sense strand siRNA: GAUCUGUGAUGGCUGCUU (SEQ ID NO: 39), antisense strand siRNA: GCAGCCAUCACAGAUCUU (residues 2556-2573 of SEQ ID NO: 6);

target sequence 39: AAATTGTTTAAGGTTGATGAA (residues 748-768 of SEQ ID NO: 5), position in gene sequence: 784, GC content: 23.8%, sense strand siRNA: AUUGUUUAAGGUUGAUGAAUU (SEQ ID NO: 40), antisense strand siRNA: UUCAUCAACCUUAAACAAUUU (residues 2517-2537 of SEQ ID NO: 6);

target sequence 40: AAGGTTGATGAATGAAACTAC (residues 757-777 of SEQ ID NO: 5), position in gene sequence: 793, GC content: 33.3%, sense strand siRNA: GGUUGAUGAAUGAAACUACUU (SEQ ID NO: 41), antisense strand siRNA: GUAGUUUCAUUCAUCAACCUU (residues 2508-2528 of SEQ ID NO: 6);

target sequence 41: AATGAAACTACTGCAGTT (residues 767-784 of SEQ ID NO: 5), position in gene sequence: 803, GC content: 28.6%, sense strand siRNA: UGAAACUACUGCAGUUUU (SEQ ID NO: 42), antisense strand siRNA: AACUGCAGUAGUUUCAUU (residues 2501-2518 of SEQ ID NO: 6);

target sequence 42: AAACTACTGCAGTTGCAC (residues 771-788 of SEQ ID NO: 5), position in gene sequence: 807, GC content: 38.1%, sense strand siRNA: ACUACUGCAGUUGCACUU (SEQ ID NO: 43), antisense strand siRNA: GUGCAACUGCAGUAGUUU (residues 2497-2514 of SEQ ID NO: 6);

target sequence 43: AATTTATAAACAGGATCTTCC (residues 799-819 of SEQ ID NO: 5), position in gene sequence: 838, GC content: 28.6%, sense strand siRNA: UUUAUAAACAGGAUCUUCCUU (SEQ ID NO: 44), antisense strand siRNA: GGAAGAUCCUGUUUAUAAAUU (residues 2466-2486 of SEQ ID NO: 6);

target sequence 44: AAACAGGATCTTCCCCCATTA (residues 806-826 of SEQ ID NO: 5), position in gene sequence: 845, GC content: 42.9%, sense strand siRNA: ACAGGAUCUUCCCCCAUUAUU (SEQ ID NO: 45), antisense strand siRNA: UAAUGGGGGAAGAUCCUGUUU (residues 2459-2479 of SEQ ID NO: 6);

target sequence 45: AAACCAAGAAATGTAGTA (residues 833-850 of SEQ ID NO: 5), position in gene sequence: 872, GC content: 23.8%, sense strand siRNA: ACCAAGAAAUGUAGUAUU (SEQ ID NO: 46), antisense strand siRNA: UACUACAUUUCUUGGUUU (residues 2435-2452 of SEQ ID NO: 6);

target sequence 46: AAGAAATGTAGTATTTAT (residues 838-855 of SEQ ID NO: 5), position in gene sequence: 877, GC content: 14.3%, sense strand siRNA: GAAAUGUAGUAUUUAUUU (SEQ ID NO: 2), antisense strand siRNA: AUAAAUACUACAUUUCUU (residues 2430-2447 of SEQ ID NO: 6);

target sequence 47: AAATGTAGTATTTATTGATAT841-861 of SEQ ID NO: 5), position in gene sequence: 883, GC content: 14.3%, sense strand siRNA: AUGUAGUAUUUAUUGAUAUUU (SEQ ID NO: 3), antisense strand siRNA: AUAUCAAUAAAUACUACAUUU (residues 2424-2444 of SEQ ID NO: 6);

target sequence 48: AACAAAGGAAAACTTAAA (residues 899-916 of SEQ ID NO: 5), position in gene sequence: 941, GC content: 19%, sense strand siRNA: CAAAGGAAAACUUAAAUU (SEQ ID NO: 4), antisense strand siRNA: UUUAAGUUUUCCUUUGUU (residues 2369-2386 of SEQ ID NO: 6);

target sequence 49: AAAGGAAAACTTAAAGTCTTG (residues 902-922 of SEQ ID NO: 5), position in gene sequence: 947, GC content: 28.6%, sense strand siRNA: AGGAAAACUUAAAGUCUUGUU (SEQ ID NO: 5), antisense strand siRNA: CAAGACUUUAAGUUUUCCUUU (residues 2363-2383 of SEQ ID NO: 6);

target sequence 50: AAAACTTAAAGTCTTGGCTAC (residues 907-927 of SEQ ID NO: 5), position in gene sequence: 952, GC content: 33.3%, sense strand siRNA: AACWUAAAGUCUUGGCUACUU (SEQ ID NO: 6), antisense strand siRNA: GUAGCCAAGACUUUAAGUUUU (residues 2358-2378 of SEQ ID NO: 6);

target sequence 51: AACTTAAAGTCTTGGCTACTA (residues 909-929 of SEQ ID NO: 5), position in gene sequence: 954, GC content: 33.3%, sense strand siRNA: CUUAAAGUCUUGGCUACUAUU (SEQ ID NO: 7), antisense strand siRNA: UAGUAGCCAAGACUUUAAGUU (residues 2356-2376 of SEQ ID NO: 6);

target sequence 52: AAAGTCTTGGCTACTACCTTT (residues 914-934 of SEQ ID NO: 5), position in gene sequence: 959, GC content: 38.1%, sense strand siRNA: AGUCUUGGCUACUACCUUUUU (SEQ ID NO: 8), antisense strand siRNA: AAAGGUAGUAGCCAAGACUUU (residues 2351-2371 of SEQ ID NO: 6);

target sequence 53: AACTTTGATGAGGCTTTA (residues 956-973 of SEQ ID NO: 5), position in gene sequence: 1001, GC content: 28.6%, sense strand siRNA: CUUUGAUGAGGCUUUAUU (SEQ ID NO: 9), antisense strand siRNA: UAAAGCCUCAUCAAAGUU (residues 2312-2329 of SEQ ID NO: 6);

target sequence 54: AAGACCAAATATAAGATAAAT (residues 998-1018 of SEQ ID NO: 5), position in gene sequence: 1046, GC content: 19%, sense strand siRNA: GACCAAAUAUAAGAUAAAUUU (SEQ ID NO: 10), antisense strand siRNA: AUUUAUCUUAUAUUUGGUCUU (residues 2267-2287 of SEQ ID NO: 6);

target sequence 55: AAATATAAGATAAATGT (residues 1004-1020 of SEQ ID NO: 5), position in gene sequence: 1052, GC content: 9.5%, sense strand siRNA: AUAUAAGAUAAAUGUUU (SEQ ID NO: 11), antisense strand siRNA: ACAUUUAUCUUAUAUUU (residues 2265-2281 of SEQ ID NO: 6);

target sequence 56: AAGATAAATGTGAAAGA (residues 1010-1026 of SEQ ID NO: 5), position in gene sequence: 1058, GC content: 19%, sense strand siRNA: GAUAAAUGUGAAAGAUU (SEQ ID NO: 12), antisense strand siRNA: UCUUUCACAUUUAUCUU (residues 2259-2275 of SEQ ID NO: 6);

target sequence 57: AAATGTGAAAGAAAACT (residues 1015-1031 of SEQ ID NO: 5), position in gene sequence: 1063, GC content: 19%, sense strand siRNA: AUGUGAAAGAAAACUUU (SEQ ID NO: 13), antisense strand siRNA: AGUUUUCUUUCACAUUU (residues 2254-2270 of SEQ ID NO: 6);

target sequence 58: AAAGAAAACTCTCGGGCCTTG (residues 1022-1042 of SEQ ID NO: 5), position in gene sequence: 1074, GC content: 47.6%, sense strand siRNA: AGAAAACUCUCGGGCCUUGUU (SEQ ID NO: 14), antisense strand siRNA: CAAGGCCCGAGAGUUUUCUUU (residues 2243-2263 of SEQ ID NO: 6);

target sequence 59: AAAACTCTCGGGCCTTGTTGC (residues 1026-1046 of SEQ ID NO: 5), position in gene sequence: 1078, GC content: 52.4%, sense strand siRNA: AACUCUCGGGCCUUGUUGCUU (SEQ ID NO: 15), antisense strand siRNA: GCAACAAGGCCCCGAGAGUUUU (residues 2239-2259 of SEQ ID NO: 6);

target sequence 60: AACTCTCGGGCCTTGTTGCGT (residues 1028-1048 of SEQ ID NO: 5), position in gene sequence: 1080, GC content: 57.1%, sense strand siRNA: CUCUCGGGCCUUGUUGCGUUU (SEQ ID NO: 16), antisense strand siRNA: ACGCAACAAGGCCCGAGAGUU (residues 2237-2257

target sequence 61: AATGTGAAAAACTAAAGAAGC (residues 1059-1079 of SEQ ID NO: 5), position in gene sequence: 1111, GC content: 28.6%, sense strand siRNA: UGUGAAAAACUAAAGAAGCCUU (SEQ ID NO: 17), antisense strand siRNA: GCUUCUUUAGUUUUUCACAUU (residues 2206-2226 of SEQ ID NO: 6);

target sequence 62: AAAAACTAAAGAAGCTA (residues 1065-1081 of SEQ ID NO: 5), position in gene sequence: 1117, GC content: 19%, sense strand siRNA: AAACUAAAGAAGCUAUU (SEQ ID NO: 18), antisense strand siRNA: UAGCUUCUUUAGUUUUU (residues 2204-2220 of SEQ ID NO: 6);

target sequence 63: AAACTAAAGAAGCTAAT (residues 1067-1083 of SEQ ID NO: 5), position in gene sequence: 1119, GC content: 19%, sense strand siRNA: ACUAAAGAAGCUAAUUU (SEQ ID NO: 19), antisense strand siRNA: AUUAGCUUCUUUAGUUU (residues 2202-2218 of SEQ ID NO: 6);

target sequence 64: AAAGAAGCTAATGAGTG (residues 1072-1088 of SEQ ID NO: 5), position in gene sequence: 1124, GC content: 28.6%, sense strand siRNA: AGAAGCUAAUGAGUGUU (SEQ ID NO: 20), antisense strand siRNA: CACUCAUUAGCUUCUUU (residues 2197-2213 of SEQ ID NO: 6);

target sequence 65: AAGCTAATGAGTGCAAA (residues 1076-1092 of SEQ ID NO: 5), position in gene sequence: 1128, GC content: 28.6%, sense strand siRNA: GCUAAUGAGUGCAAAUU (SEQ ID NO: 3), antisense strand siRNA: UUUGCACUCAUUAGCUU (residues 2193-2209 of SEQ ID NO: 6);

target sequence 66: AATGAGTGCAAATGCATCAGA (residues 1081-1101 of SEQ ID NO: 5), position in gene sequence: 1137, GC content: 38.1%, sense strand siRNA: UGAGUGCAAAUGCAUCAGAUU (SEQ ID NO: 4), antisense strand siRNA: UCUGAUGCAUUUGCACUCAUU2184-2204 of SEQ ID NO: 6);

target sequence 67: AAATGCATCAGATCTTCCATT (residues 1090-1110 of SEQ ID NO: 5), position in gene sequence: 1146, GC content: 33.3%, sense strand siRNA: AUGCAUCAGAUCUUCCAUUUU (SEQ ID NO: 5), antisense strand siRNA: AAUGGAAGAUCUGAUGCAUUU (residues 2175-2195 of SEQ ID NO: 6);

target sequence 68: AACATTGAGTGTTTCATGAAT (residues 1112-1132 of SEQ ID NO: 5), position in gene sequence: 1168, GC content: 28.6%, sense strand siRNA: CAUUGAGUGUUUCAUGAAUUU (SEQ ID NO: 6), antisense strand siRNA: AUUCAUGAAACACUCAAUGUU (residues 2153-2173 of SEQ ID NO: 6);

target sequence 69: AATGACCTTGATGTTTC (residues 1130-1146 of SEQ ID NO: 5), position in gene sequence: 1186, GC content: 28.6%, sense strand siRNA: UGACCUUGAUGUUUCUU (SEQ ID NO: 7), antisense strand siRNA: GAAACAUCAAGGUCAUU (residues 2139-2155 of SEQ ID NO: 6);

target sequence 70: AAAATGAACAGGGCTCAATTT (residues 1151-1171 of SEQ ID NO: 5), position in gene sequence: 1211, GC content: 33.3%, sense strand siRNA: AAUGAACAGGGCUCAAUUUUU (SEQ ID NO: 8), antisense strand siRNA: AAAUUGAGCCCUGUUCAUUUU (residues 2114-2134 of SEQ ID NO: 6);

target sequence 71: AATGAACAGGGCTCAATTTGA (residues 1153-1173 of SEQ ID NO: 5), position in gene sequence: 1213, GC content: 38.1%, sense strand siRNA: UGAACAGGGCUCAAUUUGAUU (SEQ ID NO: 9), antisense strand siRNA: UCAAAUUGAGCCCUGUUCAUU (residues 2112-2132 of SEQ ID NO: 6);

target sequence 72: AACAGGGCTCAATTTGAACAA (residues 1157-1177 of SEQ ID NO: 5), position in gene sequence: 1217, GC content: 38.1%, sense strand siRNA: CAGGGCUCAAUUGAACAAUU (SEQ ID NO: 10), antisense strand siRNA: UUGUUCAAAUUGAGCCCUGCUU (residues 2108-2128 of SEQ ID NO: 6);

target sequence 73: AATTTGAACAACTGTGTGCTT (residues 1167-1187 of SEQ ID NO: 5), position in gene sequence: 1227, GC content: 33.3%, sense strand siRNA: UUUGAACAACUGUGUGCUUUU (SEQ ID NO: 11), antisense strand siRNA: AAGCACACAGUUGUUCAAAUU (residues 2098-2118 of SEQ ID NO: 6);

target sequence 74: AACAACTGTGTGCTTCCCTTT (residues 1173-1193 of SEQ ID NO: 5), position in gene sequence: 1233, GC content: 42.9%, sense strand siRNA: CAACUGUGUGCUUCCCUUUUU (SEQ ID NO: 12), antisense strand siRNA: AAAGGGAAGCACACAGUUGUU (residues 2092-2112 of SEQ ID NO: 6);

target sequence 75: AACTGTGTGCTTCCCTTTTGG (residues 1176-1196 of SEQ ID NO: 5), position in gene sequence: 1236, GC content: 47.6%, sense strand siRNA: CUGUGUGCUUCCCUUUUGGUU (SEQ ID NO: 13), antisense strand siRNA: CCAAAAGGGAAGCACACAGUU (residues 2089-2109 of SEQ ID NO: 6);

target sequence 76: AACCACCTTTAAAAGCAGTAA (residues 1206-1226 of SEQ ID NO: 5), position in gene sequence: 1270, GC content: 33.3%, sense strand siRNA: CCACCUUUAAAAGCAGUAAUU (SEQ ID NO: 14), antisense strand siRNA: UUACUGCUUUUAAAGGUGGUU (residues 2059-2079 of SEQ ID NO: 6);

target sequence 77: AAAAGCAGTAATGGAACAAGC1216-1236 ofSEQ ID NO: 5), position in gene sequence: 1280, GC content: 38.1%, sense strand siRNA: AAGCAGUAAUGGAACAAGCUU (SEQ ID NO: 15), antisense strand siRNA: GCUUGUUCCAUUACUGCUUUU2049-2069 of SEQ ID NO: 6);

target sequence 78: AAGCAGTAATGGAACAAGCTA (residues 1218-1238 of SEQ ID NO: 5), position in gene sequence: 1282, GC content: 38.1%, sense strand siRNA: GCAGUAAUGGAACAAGCUAUU (SEQ ID NO: 16), antisense strand siRNA: UAGCUUGUUCCAUUACUGCUU (residues 2047-2067 ofSEQ ID NO: 6);

target sequence 79: AATGGAACAAGCTAACTTACA (residues 1225-1245 ofSEQ ID NO: 5), position in gene sequence: 1289, GC content: 33.3%, sense strand siRNA: UGGAACAAGCUAACUUACAUU (SEQ ID NO: 17), antisense strand siRNA: UGUAAGUUAGCUUGUUCCAUU (residues 2040-2060 of SEQ ID NO: 6);

target sequence 80: AACAAGCTAACTTACAACGTG (residues 1230-1250 of SEQ ID NO: 5), position in gene sequence: 1294, GC content: 38.1%, sense strand siRNA: CAAGCUAACUUACAACGUGUU (SEQ ID NO: 18), antisense strand siRNA: CACGUUGUAAGUUAGCUUGUU (residues 2035-2055 of SEQ ID NO: 6);

target sequence 81: AAGCTAACTTACAACGTGAAG (residues 1233-1253 of SEQ ID NO: 5), position in gene sequence: 1297, GC content: 38.1%, sense strand siRNA: GCUAACUUACAACGUGAAGUU (SEQ ID NO: 19), antisense strand siRNA: CUUCACGUUGUAAGUUAGCUU (residues 2032-2052 of SEQ ID NO: 6);

target sequence 82: AACTTACAACGTGAAGACATT (residues 1238-1258 of SEQ ID NO: 5), position in gene sequence: 1302, GC content: 33.3%, sense strand siRNA: CUUACAACGUGAAGACAUUUU (SEQ ID NO: 20), antisense strand siRNA: AAUGUCUUCACGUUGUAAGUU (residues 2027-2047 of SEQ ID NO: 6);

target sequence 83: AACGTGAAGACATTAGT (residues 1245-1261 of SEQ ID NO: 5), position in gene sequence: 1309, GC content: 28.6%, sense strand siRNA: CGUGAAGACAUUAGUUU (SEQ ID NO: 21), antisense strand siRNA: ACUAAUGUCUUCACGUU (residues 2024-2040 of SEQ ID NO: 6);

target sequence 84: AAGACATTAGTAGTATA (residues 1251-1267 of SEQ ID NO: 5), position in gene sequence: 1315, GC content: 19%, sense strand siRNA: GACAUUAGUAGUAUAUU (SEQ ID NO: 22), antisense strand siRNA: UAUACUACUAAUGUCUU (residues 2018-2034 of SEQ ID NO: 6);

target sequence 85: AAATTGTAGGAGGAGCAACAC (residues 1269-1289 of SEQ ID NO: 5), position in gene sequence: 1337, GC content: 42.9%, sense strand siRNA: AUUGUAGGAGGAGCAACACUU (SEQ ID NO: 23), antisense strand siRNA: GUGUUGCUCCUCCUACAAUUU (residues 1996-2016 of SEQ ID NO: 6);

target sequence 86: AACACGAATTCCTGCAGTGAA (residues 1285-1305 of SEQ ID NO: 5), position in gene sequence: 1353, GC content: 42.9%, sense strand siRNA: CACGAAUUCCUGCAGUGAAUU (SEQ ID NO: 24), antisense strand siRNA: UUCACUGCAGGAAUUCGUGUU (residues 1980-2000 of SEQ ID NO: 6);

target sequence 87: AATTCCTGCAGTGAAAGAACA (residues 1291-1311 of SEQ ID NO: 5), position in gene sequence: 1359, GC content: 38.1%, sense strand siRNA: UUCCUGCAGUGAAAGAACAUU (SEQ ID NO: 25), antisense strand siRNA: UGUUCUUUCACUGCAGGAAUU (residues 1974-1994 of SEQ ID NO: 6);

target sequence 88: AAAGAACAAATCACTAA (residues 1304-1320 of SEQ ID NO: 5), position in gene sequence: 1372, GC content: 19%, sense strand siRNA: AGAACAAAUCACUAAUU (SEQ ID NO: 26), antisense strand siRNA: UUAGUGAUUUGUUCUUU (residues 1965-1981 of SEQ ID NO: 6);

target sequence 89: AACAAATCACTAAATTC (residues 1308-1324 of SEQ ID NO: 5), position in gene sequence: 1376, GC content: 19%, sense strand siRNA: CAAAUCACUAAAUUCUU (SEQ ID NO: 27), antisense strand siRNA: GAAUUUAGUGAUUUGUU (residues 1961-1977 of SEQ ID NO: 6);

target sequence 90: AAATCACTAAATTCTTT (residues 1311-1327 of SEQ ID NO: 5), position in gene sequence: 1379, GC content: 14.3%, sense strand siRNA: AUCACUAAAUUCUUUUU (SEQ ID NO: 28), antisense strand siRNA: AAAGAAUUUAGUGAUUU (residues 1958-1974 of SEQ ID NO: 6);

target sequence 91: AAATTCTTTCTTAAAGA (residues 1319-1335 of SEQ ID NO: 5), position in gene sequence: 1387, GC content: 14.3%, sense strand siRNA: AUUCUUUCUUAAAGAUU (SEQ ID NO: 29), antisense strand siRNA: UCUUUAAGAAAGAAUUU (residues 1950-1966 of SEQ ID NO: 6);

target sequence 92: AAAGACATAAGTACCACATTA (residues 1331-1351 of SEQ ID NO: 5), position in gene sequence: 1403, GC content: 28.6%, sense strand siRNA: AGACAUAAGUACCACAUUAUU (SEQ ID NO: 30), antisense strand siRNA: UAAUGUGGUACUUAUGUCUUU (residues 1934-1954 of SEQ ID NO: 6);

target sequence 93: AAGTACCACATTAAATGCTGA1339-1359 of SEQ ID NO: 5), position in gene sequence: 1411, GC content: 33.3%, sense strand siRNA: GUACCACAUUAAAUGCUGAUU (SEQ ID NO: 31), antisense strand siRNA: UCAGCAUUUAAUGUGGUACUU1926-1946 of SEQ ID NO: 6);

target sequence 94: AAATGCTGATGAAGCTGTTGC (residues 1351-1371 of SEQ ID NO: 5), position in gene sequence: 1423, GC content: 42.9%, sense strand siRNA: AUGCUGAUGAAGCUGUUGCUU (SEQ ID NO: 32), antisense strand siRNA: GCAACAGCUUCAUCAGCAUUU (residues 1914-1934 of SEQ ID NO: 6);

target sequence 95: AAGCTGTTGCAAGAGGATG (residues 1362-1380 of SEQ ID NO: 5), position in gene sequence: 1434, GC content: 42.9%, sense strand siRNA: GCUGUUGCAAGAGGAUGUU (SEQ ID NO: 33), antisense strand siRNA: CAUCCUCUUGCAACAGCUU (residues 1905-1923 of SEQ ID NO: 6);

target sequence 96: AAGAGGATGTGCGTTAC (residues 1372-1388 of SEQ ID NO: 5), position in gene sequence: 1444, GC content: 38.1%, sense strand siRNA: GAGGAUGUGCGUUACUU (SEQ ID NO: 34), antisense strand siRNA: GUAACGCACAUCCUCUU (residues 1897-1913 of SEQ ID NO: 6);

target sequence 97: AAAGTGCGTGAATTTTCCATA (residues 1415-1435 of SEQ ID NO: 5), position in gene sequence: 1491, GC content: 33.3%, sense strand siRNA: AGUGCGUGAAUUUUCCAUAUU (SEQ ID NO: 35), antisense strand siRNA: UAUGGAAAAUUCACGCACUUU (residues 1850-1870 of SEQ ID NO: 6);

target sequence 98: AATTTTCCATAACAGAC (residues 1425-1441 of SEQ ID NO: 5), position in gene sequence: 1501, GC content: 23.8%, sense strand siRNA: UUUUCCAUAACAGACUU (SEQ ID NO: 36), antisense strand siRNA: GUCUGUUAUGGAAAAUU (residues 1844-1860 of SEQ ID NO: 6);

target sequence 99: AACAGACCTTGTTCCCT (residues 1435-1451 of SEQ ID NO: 5), position in gene sequence: 1511, GC content: 38.1%, sense strand siRNA: CAGACCUUGUUCCCUUU (SEQID NO: 37), antisense strand siRNA: AGGGAACAAGGUCUGUU (residues 1834-1850 of SEQ ID NO: 6);

target sequence 100: AATCACATTAAGGTGGAAGAC (residues 1456-1476 of SEQ ID NO: 5), position in gene sequence: 1536, GC content: 38.1%, sense strand siRNA: UCACAUUAAGGUGGAAGACUU (SEQ ID NO: 38), antisense strand siRNA: GUCUUCCACCUUAAUGUGAUU (residues 1809-1829 of SEQ ID NO: 6);

target sequence 101: AAGGTGGAAGACCTCTTTTGA (residues 1465-1485 of SEQ ID NO: 5), position in gene sequence: 1545, GC content: 42.9%, sense strand siRNA: GGUGGAAGACCUCUUUUGAUU (SEQ ID NO: 39), antisense strand siRNA: UCAAAAGAGGUCUUCCACCUU (residues 1800-1820 of SEQ ID NO: 6);

target sequence 102: AAGACCTCTTTTGAAGATGGA (residues 1472-1492 of SEQ ID NO: 5), position in gene sequence: 1552, GC content: 38.1%, sense strand siRNA: GACCUCUUUUGAAGAUGGAUU (SEQ ID NO: 40), antisense strand siRNA: UCCAUCUUCAAAAGAGGUCUU (residues 1793-1813 of SEQ ID NO: 6);

target sequence 103: AAGATGGAAGTGGGGAA (residues 1485-1501 of SEQ ID NO: 5), position in gene sequence: 1565, GC content: 38.1%, sense strand siRNA: GAUGGAAGUGGGGAAUU (SEQ ID NO: 41), antisense strand siRNA: UUCCCCACUUCCAUCUU (residues 1784-1800 of SEQ ID NO: 6);

target sequence 104: AAGTGGGGAATGTGAAG (residues 1492-1508 of SEQ ID NO: 5), position in gene sequence: 1572, GC content: 38.1%, sense strand siRNA: GUGGGGAAUGUGAAGUU (SEQ ID NO: 42), antisense strand siRNA: CUUCACAUUCCCCACUU (residues 1777-1793 of SEQ ID NO: 6);

target sequence 105: AAGTTTTCTGTAAGAACCATC (residues 1506-1526 of SEQ ID NO: 5), position in gene sequence: 1590, GC content: 33.3%, sense strand siRNA: GUUUUCUGUAAGAACCAUCUU (SEQ ID NO: 43), antisense strand siRNA: GAUGGUUCUUACAGAAAACUU (residues 1759-1779 of SEQ ID NO: 6);

target sequence 106: AAGAACCATCCTGCCCCATTC (residues 1517-1537 of SEQ ID NO: 5), position in gene sequence: 1601, GC content: 52.4%, sense strand siRNA: GAACCAUCCUGCCCCAUUCUU (SEQ ID NO: 44), antisense strand siRNA: GAAUGGGGCAGGAUGGUUCUU (residues 1748-1768 of SEQ ID NO: 6);

target sequence 107: AACCATCCTGCCCCATTCTCA (residues 1520-1540 of SEQ ID NO: 5), position in gene sequence: 1604, GC content: 52.4%, sense strand siRNA: CCAUCCUGCCCCAUUCUCAUU (SEQ ID NO: 45), antisense strand siRNA: UGAGAAUGGGGCAGGAUGGUU (residues 1745-1765 of SEQ ID NO: 6);

target sequence 108: AAAAGTCATTACTTTCCACAA (residues 1540-1560 of SEQ ID NO: 5), position in gene sequence: 1624, GC content: 28.6%, sense strand siRNA: AAGUCAUUACUUUCCACAAUU (SEQ ID NO: 46), antisense strand siRNA: UUGUGGAAAGUAAUGACUUUU (residues 1725-1745 of SEQ ID NO: 6);

target sequence 109: AAGTCATTACTTTCCACAA (residues 1542-1560 of SEQ ID NO: 5), position in gene sequence: 1626, GC content: 28.6%, sense strand siRNA: GUCAUUACUUUCCACAAUU (SEQ ID NO: 47), antisense strand siRNA: UUGUGGAAAGUAAUGACUU (residues 1725-1743 of SEQ ID NO: 6);

target sequence 110: AAGAAGGAACCATTTGA (residues 1559-1575 of SEQ ID NO: 5), position in gene sequence: 1643, GC content: 28.6%, sense strand siRNA: GAAGGAACCAUUUGAUU (SEQ ID NO: 48), antisense strand siRNA: UCAAAUGGUUCCUUCUU (residues 1710-1726 of SEQ ID NO: 6);

target sequence 111: AAGGAACCATTTGAACTAGAA (residues 1562-1582 of SEQ ID NO: 5), position in gene sequence: 1650, GC content: 33.3%, sense strand siRNA: GGAACCAUUUGAACUAGAAUU (SEQ ID NO: 49), antisense strand siRNA: UUCUAGUUCAAAUGGUUCCUU (residues 1703-1723 of SEQ ID NO: 6);

target sequence 112: AACCATTTGAACTAGAAGCAT (residues 1566-1586 of SEQ ID NO: 5), position in gene sequence: 1654, GC content: 33.3%, sense strand siRNA: CCAUUUGAACUAGAAGCAUUU (SEQ ID NO: 50), antisense strand siRNA: AUGCUUCUAGUUCAAAUGGUU1699-1719 of SEQ ID NO: 6);

target sequence 113: AACTAGAAGCATTTTATACTA (residues 1575-1595 of SEQ ID NO: 5), position in gene sequence: 1663, GC content: 23.8%, sense strand siRNA: CUAGAAGCAUUUUAUACUAUU (SEQ ID NO: 51), antisense strand siRNA: UAGUAUAAAAUGCUUCUAGUU (residues 1690-1710 of SEQ ID NO: 6);

target sequence 114: AAGCATTTTATACTAATTTAC (residues 1581-1601 of SEQ ID NO: 5), position in gene sequence: 1669, GC content: 19%, sense strand siRNA: GCAUUUUAUACUAAUUUACUU (SEQ ID NO: 52), antisense strand siRNA: GUAAAUUAGUAUAAAAUGCUU (residues 1684-1704 of SEQ ID NO: 6);

target sequence 115: AATTTACATGAAGTGCCTTAT (residues 1595-1615 of SEQ ID NO: 5), position in gene sequence: 1683, GC content: 28.6%, sense strand siRNA: UUUACAUGAAGUGCCUUAUUU (SEQ ID NO: 53), antisense strand siRNA: AUAAGGCACUUCAUGUAAAUU (residues 1670-1690 of SEQ ID NO: 6);

target sequence 116: AAGTGCCTTATCCTGAT (residues 1605-1621 of SEQ ID NO: 5), position in gene sequence: 1693, GC content: 33.3%, sense strand siRNA: GUGCCUUAUCCUGAUUU (SEQ ID NO: 54), antisense strand siRNA: AUCAGGAUAAGGCACUU (residues 1664-1680 of SEQ ID NO: 6);

target sequence 117: AAGAATTGGGAGCTTCACTAT (residues 1624-1644 of SEQ ID NO: 5), position in gene sequence: 1716, GC content: 38.1%, sense strand siRNA: GAAUUGGGAGCUUCACUAUUU (SEQ ID NO: 55), antisense strand siRNA: AUAGUGAAGCUCCCAAUUCUU (residues 1641-1661 of SEQ ID NO: 6);

target sequence 118: AATTGGGAGCTTCACTATTCA (residues 1627-1647 of SEQ ID NO: 5), position in gene sequence: 1719, GC content: 38.1%, sense strand siRNA: UUGGGAGCCUCACUAUUCAUU (SEQ ID NO: 56), antisense strand siRNA: UGAAUAGUGAAGCUCCCAAUU (residues 1638-1658 of SEQ ID NO: 6);

target sequence 119: AATGTTTTTCCACAGTCTGAT (residues 1649-1669 of SEQ ID NO: 5), position in gene sequence: 1741, GC content: 33.3%, sense strand siRNA: UGUUUUUCCACAGUCUGAUUU (SEQ ID NO: 57), antisense strand siRNA: AUCAGACUGUGGAAAAACAUU (residues 1616-1636 of SEQ ID NO: 6);

target sequence 120: AAAGTGAAGGTTAAAGTTCGT (residues 1682-1702 of SEQ ID NO: 5), position in gene sequence: 1778, GC content: 33.3%, sense strand siRNA: AGUGAAGGCUAAAGUUCGUUU (SEQ ID NO: 58), antisense strand siRNA: ACGAACUUUAACCUUCACUUU (residues 1583-1603 of SEQ ID NO: 6);

target sequence 121: AAGGTTAAAGTTCGTGTTAAC (residues 1688-1708 of SEQ ID NO: 5), position in gene sequence: 1784, GC content: 33.3%, sense strand siRNA: GGCUAAAGUCGUGCUUAACUU (SEQ ID NO: 59), antisense strand siRNA: GUUAACACGAACUUUAACCUU (residues 1577-1597 of SEQ ID NO: 6);

target sequence 122: AAAGTTCGTGTTAACATCCAT (residues 1694-1714 of SEQ ID NO: 5), position in gene sequence: 1790, GC content: 33.3%, sense strand siRNA: AGUUCGUGUUAACAUCCAUUU (SEQ ID NO: 60), antisense strand siRNA: AUGGAUGUUAACACGAACUUU (residues 1571-1591 of SEQ ID NO: 6);

target sequence 123: AACATCCATGGAATCTTCAGT (residues 1706-1726 of SEQ ID NO: 5), position in gene sequence: 1802, GC content: 38.1%, sense strand siRNA: CAUCCAUGGAAUCUUCAGUUU (SEQID NO: 61), antisense strand siRNA: ACUGAAGAUUCCAUGGAUGUU (residues 1559-1579 of SEQ ID NO: 6);

target sequence 124: AATCTTCAGTGTGGCTAGCGC (residues 1717-1737 of SEQID NO: 5), position in gene sequence: 1813, GC content: 52.4%, sense strand siRNA: UCUUCAGUGUGGCUAGCGCUU (SEQID NO: 62), antisense strand siRNA: GCGCUAGCCACACUGAAGAUU (residues 1548-1568 of SEQ ID NO: 6);

target sequence 125: AATTGAGAAGCAAAATTTGGA (residues 1744-1764 of SEQ ID NO: 5), position in gene sequence: 1844, GC content: 28.6%, sense strand siRNA: UUGAGAAGCAAAAUUUGGAUU (SEQ ID NO: 63), antisense strand siRNA: UCCAAAUUUUGCUUCUCAAUU (residues 1521-1541 of SEQ ID NO: 6);

target sequence 126: AAGCAAAATTTGGAAGGCGAT (residues 1751-1771 of SEQ ID NO: 5), position in gene sequence: 1851, GC content: 38.1%, sense strand siRNA: GCAAAAUUUGGAAGGCGAUUU (SEQID NO: 64), antisense strand siRNA: AUCGCCUUCCAAAUUUUGCUU (residues 1514-1534 of SEQ ID NO: 6);

target sequence 127: AAAATTTGGAAGGCGATCACA (residues 1755-1775 of SEQ ID NO: 5), position in gene sequence: 1855, GC content: 38.1%, sense strand siRNA: AAUUUGGAAGGCGAUCACAUU (SEQID NO: 65), antisense strand siRNA: UGUGAUCGCCUUCCAAAUUUU (residues 1510-1530 of SEQ ID NO: 6);

target sequence 128: AATTTGGAAGGCGATCACAGT (residues 1757-1777 of SEQ ID NO: 5), position in gene sequence: 1857, GC content: 42.9%, sense strand siRNA: UUUGGAAGGCGAUCACAGUUU (SEQ ID NO: 66), antisense strand siRNA: ACUGUGAUCGCCUUCCAAAUU (residues 1508-1528 of SEQ ID NO: 6);

target sequence 129: AAGGCGATCACAGTGATGCTC (residues 1764-1784 of SEQ ID NO: 5), position in gene sequence: 1864, GC content: 52.4%, sense strand siRNA: GGCGAUCACAGUGAUGCUCUU (SEQ ID NO: 67), antisense strand siRNA: GAGCAUCACUGUGAUCGCCUU (residues 1501-1521 of SEQ ID NO: 6);

target sequence 130: AATGGAGACAGAAACTT (residues 1786-1802 of SEQ ID NO: 5), position in gene sequence: 1886, GC content: 28.6%, sense strand siRNA: UGGAGACAGAAACUUUU (SEQ ID NO: 68), antisense strand siRNA: AAGUUUCUGUCUCCAUU (residues 1483-1499 of SEQ ID NO: 6);

target sequence 131: AAACTTCATTTAAAAAT (residues 1797-1813 of SEQID NO: 5), position in gene sequence: 1897, GC content: 9.5%, sense strand siRNA: ACUUCAUUUAAAAAUUU (SEQID NO: 69), antisense strand siRNA: AUUUUUAAAUGAAGUUU (residues 1472-1488 of SEQ ID NO: 6);

target sequence 132: AAAAATGAAAACAAAGATAAT (residues 1808-1828 of SEQ ID NO: 5), position in gene sequence: 1912, GC content: 14.3%, sense strand siRNA: AAAUGAAAACAAAGAUAAUUU (SEQ ID NO: 70), antisense strand siRNA: AUUAUCUUUGUUUUCAUUUUU (residues 1457-1477 of SEQ ID NO: 6);

target sequence 133: AAATGAAAACAAAGATAATAT (residues 1810-1830 of SEQ ID NO: 5), position in gene sequence: 1914, GC content: 14.3%, sense strand siRNA: AUGAAAACAAAGAUAAUAUUU (SEQ ID NO: 71), antisense strand siRNA: AUAUUAUCUUUGUUUUCAUUU (residues 1455-1475 of SEQ ID NO: 6);

target sequence 134: AAAACAAAGATAATATGGATA (residues 1815-1835 of SEQ ID NO: 5), position in gene sequence: 1919, GC content: 19%, sense strand siRNA: AACAAAGAUAAUAUGGAUAUU (SEQ ID NO: 72), antisense strand siRNA: UAUCCAUAUUAUCUUUGUUUU (residues 1450-1470 of SEQ ID NO: 6);

target sequence 135: AACAAAGATAATATGGATAAA (residues 1817-1837 of SEQ ID NO: 5), position in gene sequence: 1921, GC content: 19%, sense strand siRNA: CAAAGAUAAUAUGGAUAAAUU (SEQ ID NO: 73), antisense strand siRNA: UUUAUCCAUAUUAUCUUUGUU (residues 1448-1468 of SEQ ID NO: 6);

target sequence 136: AAAGATAATATGGATAAAATG (residues 1820-1840 of SEQ ID NO: 5), position in gene sequence: 1924, GC content: 19%, sense strand siRNA: AGAUAAUAUGGAUAAAAUGUU (SEQ ID NO: 74), antisense strand siRNA: CAUUUUAUCCAUAUUAUCUUU (residues 1445-1465 of SEQ ID NO: 6);

target sequence 137: AATATGGATAAAATGCAGGTT (residues 1829-1846 of SEQ ID NO: 5), position in gene sequence: 1930, GC content: 28.6%, sense strand siRNA: UAUGGAUAAAAUGCAGGUUUU (SEQ ID NO: 75), antisense strand siRNA: AACCUGCAUUUUAUCCAUAUU (residues 1439-1459 of SEQ ID NO: 6);

target sequence 138: AAAATGCAGGTTGATCAAGAA (residues 1835-1855 of SEQ ID NO: 5), position in gene sequence: 1939, GC content: 33.3%, sense strand siRNA: AAUGCAGGUUGAUCAAGAAUU (SEQ ID NO: 76), antisense strand siRNA: UUCUUGAUCAACCUGCAUUUU (residues 1430-1450 of SEQ ID NO: 6);

target sequence 139: AATGCAGGTTGATCAAGAAGA (residues 1837-1857 of SEQ ID NO: 5), position in gene sequence: 1941, GC content: 38.1%, sense strand siRNA: UGCAGGUUGAUCAAGAAGAUU (SEQID NO: 77), antisense strand siRNA: UCUUCUCGAUCAACCUGCAUU (residues 1428-1448 of SEQ ID NO: 6);

target sequence 140: AAGAAGAAGGGCATCAA (residues 1851-1867 of SEQ ID NO: 5), position in gene sequence: 1955, GC content: 33.3%, sense strand siRNA: GAAGAAGGGCAUCAAUU (SEQ ID NO: 78), antisense strand siRNA: UUGAUGCCCUUCUUCUU (residues 1418-1434 of SEQ ID NO: 6);

target sequence 141: AAGAAGGGCATCAAAAA (residues 1854-1870 of SEQ ID NO: 5), position in gene sequence: 1958, GC content: 28.6%, sense strand siRNA: GAAGGGCAUCAAAAAUU (SEQ ID NO: 79), antisense strand siRNA: UUUUUGAUGCCCUUCUU (residues 1415-1431 of SEQ ID NO: 6);

target sequence 142: AAGGGCATCAAAAATGT (residues 1857-1873 of SEQ ID NO: 5), position in gene sequence: 1961, GC content: 28.6%, sense strand siRNA: GGGCAUCAAAAAUGUUU (SEQ ID NO: 80), antisense strand siRNA: ACAUUUUUGAUGCCCUU (residues 1412-1428 of SEQ ID NO: 6);

target sequence 143: AAAAATGTCATGCTGAACACA (residues 1866-1886 of SEQ ID NO: 5), position in gene sequence: 1974, GC content: 33.3%, sense strand siRNA: AAAUGUCAUGCUGAACACAUU (SEQ ID NO: 81), antisense strand siRNA: UGUGCUCAGCAUGACAUUUUU (residues 1399-1419 of SEQ ID NO: 6);

target sequence 144: AAATGTCATGCTGAACACACT (residues 1868-1888 of SEQ ID NO: 5), position in gene sequence: 1976, GC content: 38.1%, sense strand siRNA: AUGUCAUGCUGAACACACUUU (SEQID NO: 82), antisense strand siRNA: AGUGUGUUCAGCAUGACAUUU (residues 1397-1417 of SEQ ID NO: 6);

target sequence 145: AACACACTCCAGAAGAGGAAA (residues 1881-1901 of SEQ ID NO: 5), position in gene sequence: 1989, GC content: 42.9%, sense strand siRNA: CACACUCCAGAAGAGGAAAUU (SEQ ID NO: 83), antisense strand siRNA: UUUCCUCUUCUGGAGUGUGUU (residues 1384-1404 of SEQ ID NO: 6);

target sequence 146: AAGAGGAAATTGATCATACAG (residues 1893-1913 of SEQ ID NO: 5), position in gene sequence: 2001, GC content: 33.3%, sense strand siRNA: GAGGAAAUUGAUCAUACAGUU (SEQ ID NO: 84), antisense strand siRNA: CUGUAUGAUCAAUUUCCUCUU (residues 1372-1392 of SEQ ID NO: 6);

target sequence 147: AAATTGATCATACAGGAGCCA (residues 1899-1919 of SEQ ID NO: 5), position in gene sequence: 2007, GC content: 38.1%, sense strand siRNA: AUUGAUCAUACAGGAGCCAUU (SEQ ID NO: 85), antisense strand siRNA: UGGCUCCUGUAUGAUCAAUUU (residues 1366-1386 of SEQ ID NO: 6);

target sequence 148: AAAACAAAGTCAGCTGT (residues 1919-1935 of SEQ ID NO: 5), position in gene sequence: 2027, GC content: 28.6%, sense strand siRNA: AACAAAGUCAGCUGUUU (SEQ ID NO: 86), antisense strand siRNA: ACAGCUGACUUUGUUUU (residues 1350-1366 of SEQ ID NO: 6);

target sequence 149: AACAAAGTCAGCTGTCTCAGA (residues 1921-1941 of SEQ ID NO: 5), position in gene sequence: 2033, GC content: 42.9%, sense strand siRNA: CAAAGUCAGCUGUCUCAGAUU (SEQ ID NO: 87), antisense strand siRNA: UCUGAGACAGCUGACUUUGUU (residues 1344-1364 of SEQ ID NO: 6);

target sequence 150: AAAGTCAGCTGTCTCAGACAA (residues 1924-1944 of SEQ ID NO: 5), position in gene sequence: 2036, GC content: 42.9%, sense strand siRNA: AGUCAGCUGUCUCAGACAAUU (SEQ ID NO: 88), antisense strand siRNA: UUGUCUGAGACAGCUGACUUU (residues 1341-1361 of SEQ ID NO: 6);

target sequence 151: AAACAAGACCGATTAAATCAG (residues 1943-1963 of SEQ ID NO: 5), position in gene sequence: 2055, GC content: 33.3%, sense strand siRNA: ACAAGACCGAUUAAAUCAGUU (SEQ ID NO: 89), antisense strand siRNA: CUGAUUUAAUCGGUCUUGUUU (residues 1322-1342 of SEQ ID NO: 6);

target sequence 152: AAGACCGATTAAATCAGACAC (residues 1947-1967 of SEQ ID NO: 5), position in gene sequence: 2059, GC content: 38.1%, sense strand siRNA: GACCGAUUAAAUCAGACACUU (SEQ ID NO: 90), antisense strand siRNA: GUGUCUGAUUUAAUCGGUCUU (residues 1318-1338 of SEQ ID NO: 6);

target sequence 153: AAATCAGACACTTAAAAAAGG (residues 1957-977 of SEQ ID NO: 5), position in gene sequence: 2069, GC content: 28.6%, sense strand siRNA: AUCAGACACUUAAAAAAGGUU (SEQ ID NO: 91), antisense strand siRNA: CCUUUUUUAAGUGUCUGAUUU1308-1328 of SEQ ID NO: 6);

target sequence 154: AAAAAAGGAAAAGTCAA (residues 1970-1986 of SEQ ID NO: 5), position in gene sequence: 2082, GC content: 19%, sense strand siRNA: AAAAGGAAAAGUCAAUU (SEQ ID NO: 92), antisense strand siRNA: UUGACUUUUCCUUUUUU (residues 1299-1315 of SEQ ID NO: 6);

target sequence 155: AAAAGGAAAAGTCAAAA (residues 1972-1988 of SEQ ID NO: 5), position in gene sequence: 2084, GC content: 19%, sense strand siRNA: AAGGAAAAGUCAAAAUU (SEQ ID NO: 93), antisense strand siRNA: UUUUGACUUUUCCUUUU (residues 1297-1313 of SEQ ID NO: 6);

target sequence 156: AAGGAAAAGTCAAAAGT (residues 1974-1990 of SEQ ID NO: 5), position in gene sequence: 2086, GC content: 23.8%, sense strand siRNA: GGAAAAGUCAAAAGUUU (SEQ ID NO: 94), antisense strand siRNA: ACUUUUGACUUUUCCUU (residues 1295-1311 of SEQ ID NO: 6);

target sequence 157: AAAAGTCAAAAGTATTG (residues 1978-1994 of SEQ ID NO: 5), position in gene sequence: 2090, GC content: 19%, sense strand siRNA: AAGUCAAAAGUAUUGGUU (SEQ ID NO: 95), antisense strand siRNA: CAAUACUUUUGACUUUU (residues 1291-1307 of SEQ ID NO: 6);

target sequence 158: AAAAGTATTGATCTACCGATC (residues 1985-2005 of SEQ ID NO: 5), position in gene sequence: 2101, GC content: 33.3%, sense strand siRNA: AAGUAUUGAUCUACCGAUCUU (SEQ ID NO: 96), antisense strand siRNA: GAUCGGUAGAUCAAUACUUUU (residues 1280-1300 of SEQ ID NO: 6);

target sequence 159: AAGTATTGATCTACCGATCCA (residues 1987-2007 of SEQ ID NO: 5), position in gene sequence: 2103, GC content: 38.1%, sense strand siRNA: GUAUUGAUCUACCGAUCCAUU (SEQ ID NO: 97), antisense strand siRNA: UGGAUCGGUAGAUCAAUACUU (residues 1278-1298 of SEQ ID NO: 6);

target sequence 160: AACTAGGCCAAGATCTT (residues 2025-2041 of SEQ ID NO: 5), position in gene sequence: 2141, GC content: 33.3%, sense strand siRNA: CUAGGCCAAGAUCUUUU (SEQ ID NO: 98), antisense strand siRNA: AAGAUCUUGGCCUAGUU (residues 1244-1260 of SEQ ID NO: 6);

target sequence 161: AAGATCTTCTCAACAGC (residues 2034-2050 of SEQ ID NO: 5), position in gene sequence: 2150, GC content: 33.3%, sense strand siRNA: GAUCUUCUCAACAGCUU (SEQ ID NO: 99), antisense strand siRNA: GCUGUUGAGAAGAUCUU (residues 1235-1251 of SEQ ID NO: 6);

target sequence 162: AACAGCTACATTGAAAATGAG (residues 2045-2065 of SEQ ID NO: 5), position in gene sequence: 2165, GC content: 33.3%, sense strand siRNA: CAGCUACAUUGAAAAUGAGUU (SEQ ID NO: 100), antisense strand siRNA: CUCAUUUUCAAUGUAGCUGUU (residues 1220-1240 of SEQ ID NO: 6);

target sequence 163: AAAATGAGGGGAAGATGATCA (residues 2058-2078 of SEQ ID NO: 5), position in gene sequence: 2178, GC content: 38.1%, sense strand siRNA: AAUGAGGGGAAGAUGAUCAUU (SEQ ID NO: 101), antisense strand siRNA: UGAUCAUCUUCCCCUCAUUUU (residues 1207-1227 of SEQ ID NO: 6);

target sequence 164: AATGAGGGGAAGATGATCATG (residues 2060-2080 of SEQ ID NO: 5), position in gene sequence: 2180, GC content: 42.9%, sense strand siRNA: UGAGGGGAAGAUGAUCAUGUU (SEQ ID NO: 102), antisense strand siRNA: CAUGAUCAUCUUCCCCUCAUU (residues 1205-1225 of SEQ ID NO: 6);

target sequence 165: AAGATGATCATGCAAGATAAG (residues 2069-2089 of SEQ ID NO: 5), position in gene sequence: 2189, GC content: 33.3%, sense strand siRNA: GAUGAUCAUGCAAGAUAAGUU (SEQ ID NO: 103), antisense strand siRNA: CUUAUCUUGCAUGAUCAUCUU (residues 1196-1216 of SEQ ID NO: 6);

target sequence 166: AAGATAAGTTAGAGAAAGA (residues 2082-2100 of SEQ ID NO: 5), position in gene sequence: 2202, GC content: 23.8%, sense strand siRNA: GAUAAGUUAGAGAAAGAUU (SEQ ID NO: 104), antisense strand siRNA: UCUUUCUCUAACUUAUCUU (residues 1185-1203 of SEQ ID NO: 6);

target sequence 167: AAGTTAGAGAAAGAAAG (residues 2087-2103 of SEQ ID NO: 5), position in gene sequence: 2207, GC content: 23.8%, sense strand siRNA: GUUAGAGAAAGAAAGUU (SEQ ID NO: 105), antisense strand siRNA: CUUUCUUUCUCUAACUU (residues 1182-1198 of SEQ ID NO: 6);

target sequence 168: AAAGAAAGAAATGATGC (residues 2096-2112 of SEQ ID NO: 5), position in gene sequence: 2216, GC content: 23.8%, sense strand siRNA: AGAAAGAAAUGAUGCUU (SEQ ID NO: 106), antisense strand siRNA: GCAUCAUUUCUUUCUUU (residues 1173-1189 of SEQ ID NO: 6);

target sequence 169: AAGAAATGATGCTAAGAATGC (residues 2102-2121 of SEQ ID NO: 5), position in gene sequence: 2225, GC content: 33.3%, sense strand siRNA: GAAAUGAUGCUAAGAAUGCUU (SEQ ID NO: 107), antisense strand siRNA: GCAUUCUUAGCAUCAUUUCUU (residues 1164-1184 of SEQ ID NO: 6);

target sequence 170: AAATGATGCTAAGAATGCCGT (residues 2104-2124 of SEQ ID NO: 5), position in gene sequence: 2228, GC content: 38.1%, sense strand siRNA: AUGAUGCUAAGAAUGCCGUUU (SEQ ID NO: 108), antisense strand siRNA: ACGGCAUUCUUAGCAUCAUUU (residues 1161-1181 of SEQ ID NO: 6);

target sequence 171: AAGAATGCCGTTGAAGAATAT (residues 2114-2134 of SEQ ID NO: 5), position in gene sequence: 2238, GC content: 33.3%, sense strand siRNA: GAAUGCCGUUGAAGAAUAUUU (SEQ ID NO: 109), antisense strand siRNA: AUAUUCUUCAACGGCAUUCUU (residues 1151-1171 of SEQ ID NO: 6);

target sequence 172: AATGCCGTTGAAGAATATGTA (residues 2117-2137 of SEQ ID NO: 5), position in gene sequence: 2241, GC content: 33.3%, sense strand siRNA: UGCCGUUGAAGAAUAUGUAUU (SEQ ID NO: 110), antisense strand siRNA: UACAUAUUCUUCAACGGCAUU (residues 1148-1168 of SEQ ID NO: 6);

target sequence 173: AAGAATATGTATATGATTTTA (residues 2127-2147 of SEQ ID NO: 5), position in gene sequence: 2251, GC content: 14.3%, sense strand siRNA: GAAUAUGUAUAUGAUUUUAUU (SEQ ID NO: 111), antisense strand siRNA: UAAAAUCAUAUACAUAUUCUU (residues 1138-1158 of SEQ ID NO: 6);

target sequence 174: AATATGTATATGATTTTAGAG (residues 2130-2150 of SEQ ID NO: 5), position in gene sequence: 2254, GC content: 19%, sense strand siRNA: UAUGUAUAUGAUUUUAGAGUU (SEQ ID NO: 112), antisense strand siRNA: CUCUAAAAUCAUAUACAUAUU (residues 1135-1155 of SEQ ID NO: 6);

target sequence 175: AAAAATTCATCACTCCAGAAG (residues 2172-2192 of SEQ ID NO: 5), position in gene sequence: 2300, GC content: 33.3%, sense strand siRNA: AAAUUCAUCACUCCAGAAGUU (SEQ ID NO: 113), antisense strand siRNA: CUUCUGGAGUGAUGAAUUUUU (residues 1093-1113 of SEQ ID NO: 6);

target sequence 176: AAATTCATCACTCCAGAAGAC (residues 2174-2194 of SEQ ID NO: 5), position in gene sequence: 2302, GC content: 38.1%, sense strand siRNA: AUUCAUCACUCCAGAAGACUU (SEQ ID NO: 114), antisense strand siRNA: GUCUUCUGGAGUGAUGAAUUU (residues 1091-1111 of SEQ ID NO: 6);

target sequence 177: AAGACTTGAGTAAACTGTCTG (residues 2190-2210 of SEQ ID NO: 5), position in gene sequence: 2318, GC content: 38.1%, sense strand siRNA: GACUUGAGUAAACUGUCUGUU (SEQ ID NO: 115), antisense strand siRNA: CAGACAGUUUACUCAAGUCUU (residues 1075-1095 of SEQ ID NO: 6);

target sequence 178: AAACTGTCTGCAGTATTGGA (residues 2201-2220 of SEQ ID NO: 5), position in gene sequence: 2329, GC content: 38.1%, sense strand siRNA: ACUGUCUGCAGUAUUGGAUU (SEQ ID NO: 116), antisense strand siRNA: UCCAAUACUGCAGACAGUUU (residues 1065-1084 of SEQ ID NO: 6);

target sequence 179: AAAATTGGCTTTATGAAGACG (residues 2229-2249 of SEQ ID NO: 5), position in gene sequence: 2361, GC content: 33.3%, sense strand siRNA: AAUUGGCUUUAUGAAGACGUU (SEQ ID NO: 117), antisense strand siRNA: CGUCUUCAUAAAGCCAAUUUU (residues 1036-1056 of SEQ ID NO: 6);

target sequence 180: AATTGGCTTTATGAAGACGGA (residues 2231-2251 of SEQ ID NO: 5), position in gene sequence: 2363, GC content: 38.1%, sense strand siRNA: UUGGCUUUAUGAAGACGGAUU (SEQ ID NO: 118), antisense strand siRNA: UCCGUCUUCAUAAAGCCAAUU (residues 1034-1054 of SEQ ID NO: 6);

target sequence 181: AAGACGGAGAGGACCAACCTA (residues 2244-2264 of SEQ ID NO: 5), position in gene sequence: 2376, GC content: 52.4%, sense strand siRNA: GACGGAGAGGACCAACCUAUU (SEQ ID NO: 119), antisense strand siRNA: UAGGUUGGUCCUCUCCGUCUU (residues 1021-1041 of SEQ ID NO: 6);

target sequence 182: AACCTAAACAAGTTTATGTGG (residues 2259-2279 of SEQ ID NO: 5), position in gene sequence: 2391, GC content: 33.3%, sense strand siRNA: CCUAAACAAGUUUAUGUGGUU (SEQ ID NO: 120), antisense strand siRNA: CCACAUAAACUUGUUUAGGUU (residues 1006-1026 of SEQ ID NO: 6);

target sequence 183: AAACAAGTTTATGTGGA (residues 2264-2280 of SEQ ID NO: 5), position in gene sequence: 2396, GC content: 23.8%, sense strand siRNA: ACAAGUUUAUGUGGAUU (SEQ ID NO: 12 1), antisense strand siRNA: UCCACAUAAACUUGUUU (residues 1005-1021 of SEQ ID NO: 6);

target sequence 184: AAGTTTATGTGGATAAG (residues 2268-2284 of SEQ ID NO: 5), position in gene sequence: 2400, GC content: 23.8%, sense strand siRNA: GUUUAUGUGGAUAAGUU (SEQ ID NO: 122), antisense strand siRNA: CUUAUCCACAUAAACUU (residues 1001-1017 of SEQ ID NO: 6);

target sequence 185: AAGCTTCAAGAACTAAAGAAA (residues 2282-2302 of SEQ ID NO: 5), position in gene sequence: 2418, GC content: 28.6%, sense strand siRNA: GCUUCAAGAACUAAAGAAAUU (SEQ ID NO: 123), antisense strand siRNA: UUUCUUUAGUUCUUGAAGCUU (residues 983-1003 of SEQ ID NO: 6);

target sequence 186: AAGAACTAAAGAAATACGGCC (residues 2289-2309 of SEQ ID NO: 5), position in gene sequence: 2425, GC content: 38.1%, sense strand siRNA: GAACUAAAGAAAUACGGCCUU (SEQ ID NO: 124), antisense strand siRNA: GGCCGUAUUUCUUUAGUUCUU (residues 976-996 of SEQ ID NO: 6);

target sequence 187: AACTAAAGAAATACGGCCAGC (residues 2292-2312 of SEQ ID NO: 5), position in gene sequence: 2428, GC content: 42.9%, sense strand siRNA: CUAAAGAAAUACGGCCAGCUU (SEQ ID NO: 125), antisense strand siRNA: GCUGGCCGUAUUUCUUUAGUU (residues 973-993 of SEQ ID NO: 6);

target sequence 188: AAAGAAATACGGCCAGCCTAT (residues 2296-2316 of SEQ ID NO: 5), position in gene sequence: 2432, GC content: 42.9%, sense strand siRNA: AGAAAUACGGCCAGCCUAUUU (SEQ ID NO: 126), antisense strand siRNA: AUAGGCUGGCCGUAUUUCUUU (residues 969-989 of SEQ ID NO: 6);

target sequence 189: AAATACGGCCAGCCTATTCAA (residues 2300-2320 of SEQ ID NO: 5), position in gene sequence: 2436, GC content: 42.9%, sense strand siRNA: AUACGGCCAGCCUAUUCAAUU (SEQ ID NO: 127), antisense strand siRNA: UUGAAUAGGCUGGCCGUAUUU (residues 965-985 of SEQ ID NO: 6);

target sequence 190: AAATGAAGTACATGGAGCATG (residues 2319-2339 of SEQ ID NO: 5), position in gene sequence: 2455, GC content: 38.1%, sense strand siRNA: AUGAAGUACAUGGAGCAUGUU (SEQ ID NO: 128), antisense strand siRNA: CAUGCUCCAUGUACUUCAUUU (residues 946-966 of SEQ ID NO: 6);

target sequence 191: AAGTACATGGAGCATGA (residues 2324-2340 of SEQ ID NO: 5), position in gene sequence: 2460, GC content: 33.3%, sense strand siRNA: GUACAUGGAGCAUGAUU (SEQ ID NO: 129), antisense strand siRNA: UCAUGCUCCAUGUACUU (residues 945-961 of SEQ ID NO: 6);

target sequence 192: AAAAGCCTTAAATGACTTGGG (residues 2350-2370 of SEQ ID NO: 5), position in gene sequence: 2490, GC content: 38.1%, sense strand siRNA: AAGCCUUAAAUGACUUGGGUU (SEQ ID NO: 130), antisense strand siRNA: CCCAAGUCAUUUAAGGCUUUU (residues 915-935 of SEQ ID NO: 6);

target sequence 193: AAGCCTTAAATGACTTGGGAA (residues 2352-2372 of SEQ ID NO: 5), position in gene sequence: 2492, GC content: 38.1%, sense strand siRNA: GCCUUAAAUGACUUGGGAAUU (SEQ ID NO: 131), antisense strand siRNA: UUCCCAAGUCAUUUAAGGCUU (residues 913-933 of SEQ ID NO: 6);

target sequence 194: AAATGACTTGGGAAAAAAGAT (residues 2359-2379 of SEQ ID NO: 5), position in gene sequence: 2499, GC content: 28.6%, sense strand siRNA: AUGACUUGGGAAAAAAGAUUU (SEQ ID NO: 132), antisense strand siRNA: AUCUUUUUUCCCAAGUCAUUU (residues 906-926 of SEQ ID NO: 6);

target sequence 195: AAAAAAGATCCAACTTGTCAT (residues 2371-2391 of SEQ ID NO: 5), position in gene sequence: 2511, GC content: 28.6%, sense strand siRNA: AAAAGAUCCAACUUGUCAUUU (SEQ ID NO: 133), antisense strand siRNA: AUGACAAGUUGGAUCUUUUUU (residues 894-914 of SEQ ID NO: 6);

target sequence 196: AAAAGATCCAACTTGTCATGA (residues 2373-2393 of SEQ ID NO: 5), position in gene sequence: 2513, GC content: 33.3%, sense strand siRNA: AAGAUCCAACUUGUCAUGAUU (SEQ ID NO: 134), antisense strand siRNA: UCAUGACAAGUUGGAUCUUUU (residues 892-912 of SEQ ID NO: 6);

target sequence 197: AAGATCCAACTTGTCATGAAA (residues 2375-2395 of SEQ ID NO: 5), position in gene sequence: 2515, GC content: 33.3%, sense strand siRNA: GAUCCAACUUGUCAUGAAAUU (SEQ ID NO: 135), antisense strand siRNA: UUUCAUGACAAGUUGGAUCUU (residues 890-910 of SEQ ID NO: 6);

target sequence 198: AACTTGTCATGAAAGTGAT (residues 2382-2400 of SEQ ID NO: 5), position in gene sequence: 2522, GC content: 28.6%, sense strand siRNA: CUUGUCAUGAAAGUGAUUU (SEQ ID NO: 136), antisense strand siRNA: AUCACUUUCAUGACAAGUU (residues 885-903 of SEQ ID NO: 6);

target sequence 199: AAAGTGATAGAAGCTTA (residues 2393-2409 of SEQ ID NO: 5), position in gene sequence: 2533, GC content: 23.8%, sense strand siRNA: AGUGAUAGAAGCUUAUU (SEQ ID NO: 137), antisense strand siRNA: UAAGCUUCUAUCACUUU (residues 876-902 of SEQ ID NO: 6);

target sequence 200: AAGCTTATAGAAACAAGGATG (residues 2403-2423 of SEQ ID NO: 5), position in gene sequence: 2547, GC content: 33.3%, sense strand siRNA: GCUUAUAGAAACAAGGAUGUU (SEQ ID NO: 138), antisense strand siRNA: CAUCCUUGUUUCUAUAAGCUU (residues 862-882 of SEQ ID NO: 6);

target sequence 201: AAACAAGGATGAAAGATATGA (residues 2413-2433 of SEQ ID NO: 5), position in gene sequence: 2557, GC content: 28.6%, sense strand siRNA: ACAAGGAUGAAAGAUAUGAUU (SEQ ID NO: 139), antisense strand siRNA: UCAUAUCUUUCAUCCUUGUUU (residues 852-872 of SEQ ID NO: 6);

target sequence 202: AAGGATGAAAGATATGATCAT (residues 2417-2437 of SEQ ID NO: 5), position in gene sequence: 2561, GC content: 28.6%, sense strand siRNA: GGAUGAAAGAUAUGAUCAUUU (SEQ ID NO: 140), antisense strand siRNA: AUGAUCAUAUCUUUCAUCCUU (residues 848-868 of SEQ ID NO: 6);

target sequence 203: AAAGATATGATCATCTGGATC (residues 2424-2444 of SEQ ID NO: 5), position in gene sequence: 2568, GC content: 33.3%, sense strand siRNA: AGAUAUGAUCAUCUGGAUCUU (SEQ ID NO: 141), antisense strand siRNA: GAUCCAGAUGAUCAUAUCUUU (residues 841-861 of SEQ ID NO: 6);

target sequence 204: AAATGGAAAAGGTTGAA (residues 2451-2467 of SEQ ID NO: 5), position in gene sequence: 2595, GC content: 23.8%, sense strand siRNA: AUGGAAAAGGUUGAAUU (SEQ ID NO: 142), antisense strand siRNA: UUCAACCUUUUCCAUUU (residues 818-834 of SEQ ID NO: 6);

target sequence 205: AAAAGGTTGAAAAATGT (residues 2457-2473 of SEQ ID NO: 5), position in gene sequence: 2601, GC content: 19%, sense strand siRNA: AAGGUUGAAAAAUGUUU (SEQ ID NO: 143), antisense strand siRNA: ACAUUUUUCAACCUUUU (residues 812-828 of SEQ ID NO: 6);

target sequence 206: AAGGTTGAAAAATGTAT (residues 2459-2475 of SEQ ID NO: 5), position in gene sequence: 2603, GC content: 19%, sense strand siRNA: GGUUGAAAAAUGUAUUU (SEQ ID NO: 144), antisense strand siRNA: AUACAUUUUUCAACCUU (residues 810-826 of SEQ ID NO: 6);

target sequence 207: AAAAATGTATCAGTGATGCCA (residues 2466-2486 of SEQ ID NO: 5), position in gene sequence: 2614, GC content: 33.3%, sense strand siRNA: AAAUGUAUCAGUGAUGCCAUU (SEQ ID NO: 145), antisense strand siRNA: UGGCAUCACUGAUACAUUUUU (residues 799-819 of SEQ ID NO: 6);

target sequence 208: AAATGTATCAGTGATGCCATG (residues 2468-2488 of SEQ ID NO: 5), position in gene sequence: 2616, GC content: 38.1%, sense strand siRNA: AUGUAUCAGUGAUGCCAUGUU (SEQ ID NO: 146), antisense strand siRNA: CAUGGCAUCACUGAUACAUUU (residues 797-817 of SEQ ID NO: 6);

target sequence 209: AATAGTAAGATGAATGCACAG (residues 2498-2518 of SEQ ID NO: 5), position in gene sequence: 2646, GC content: 33.3%, sense strand siRNA: UAGUAAGAUGAAUGCACAGUU (SEQ ID NO: 147), antisense strand siRNA: CUGUGCAUUCAUCUUACUAUU (residues 767-787 of SEQ ID NO: 6);

target sequence 210: AAGATGAATGCACAGAA (residues 2504-2520 of SEQ ID NO: 5), position in gene sequence: 2652, GC content: 28.6%, sense strand siRNA: GAUGAAUGCACAGAAUU (SEQ ID NO: 148), antisense strand siRNA: UUCUGUGCAUUCAUCUU (residues 765-781 of SEQ ID NO: 6);

target sequence 211: AATGCACAGAACAAACT (residues 2510-2526 of SEQ ID NO: 5), position in gene sequence: 2658, GC content: 28.6%, sense strand siRNA: UGCACAGAACAAACUUU (SEQ ID NO: 149), antisense strand siRNA: AGUUUGUUCUGUGCAUU (residues 759-775 of SEQ ID NO: 6);

target sequence 212: AACAAACTAAGTCTCAC (residues 2519-2535 of SEQ ID NO: 5), position in gene sequence: 2667, GC content: 28.6%, sense strand siRNA: CAAACUAAGUCUCACUU (SEQ ID NO: 150), antisense strand siRNA: GUGAGACUUAGUUUGUU (residues 750-766 of SEQ ID NO: 6);

target sequence 213: AAACTAAGTCTCACTCAAGAT (residues 2522-2542 of SEQ ID NO: 5), position in gene sequence: 2674, GC content: 33.3%, sense strand siRNA: ACUAAGUCUCACUCAAGAUUU (SEQ ID NO: 151), antisense strand siRNA: AUCUUGAGUGAGACUUAGUUU (residues 743-763 of SEQ ID NO: 6);

target sequence 214: AAGTCTCACTCAAGATCCTGT (residues 2527-2547 of SEQ ID NO: 5), position in gene sequence: 2679, GC content: 42.9%, sense strand siRNA: GUCUCACUCAAGAUCCUGUUU (SEQ ID NO: 152), antisense strand siRNA: ACAGGAUCUUGAGUGAGACUU (residues 738-758 of SEQ ID NO: 6);

target sequence 215: AAGATCCTGTGGTAAAAGTTT (residues 2538-2558 of SEQ ID NO: 5), position in gene sequence: 2690, GC content: 33.3%, sense strand siRNA: GAUCCUGUGGUAAAAGUUUUU (SEQ ID NO: 153), antisense strand siRNA: AAACUUUUACCACAGGAUCUU (residues 727-747 of SEQ ID NO: 6);

target sequence 216: AAAAGTTTCAGAAATAGTAGC (residues 2551-2571 of SEQ ID NO: 5), position in gene sequence: 2703, GC content: 28.6%, sense strand siRNA: AAGUUUCAGAAAUAGUAGCUU (SEQ ID NO: 154), antisense strand siRNA: GCUACUAUUUCUGAAACUUUU (residues 714-734 of SEQ ID NO: 6);

target sequence 217: AAGTTTCAGAAATAGTAGCAA (residues 2553-2573 of SEQ ID NO: 5), position in gene sequence: 2705, GC content: 28.6%, sense strand siRNA: GUUUCAGAAAUAGUAGCAAUU (SEQ ID NO: 155), antisense strand siRNA: UUGCUACUAUUUCUGAAACUU (residues 712-732 of SEQ ID NO: 6);

target sequence 218: AAATAGTAGCAAAGTCAAA (residues 2562-2580 of SEQ ID NO: 5), position in gene sequence: 2714, GC content: 23.8%, sense strand siRNA: AUAGUAGCAAAGUCAAAUU (SEQ ID NO: 156), antisense strand siRNA: UUUGACUUUGCUACUAUUU (residues 705-723 of SEQ ID NO: 6);

target sequence 219: AAAGTCAAAGGAACTGG (residues 2572-2588 of SEQ ID NO: 5), position in gene sequence: 2724, GC content: 33.3%, sense strand siRNA: AGUCAAAGGAACUGGUU (SEQ ID NO: 157), antisense strand siRNA: CCAGUUCCUUUGACUUU (residues 697-713 of SEQ ID NO: 6);

target sequence 220: AAAGGAACTGGATAATT (residues 2578-2594 of SEQ ID NO: 5), position in gene sequence: 2730, GC content: 23.8%, sense strand siRNA: AGGAACUGGAUAAUUUU (SEQ ID NO: 158), antisense strand siRNA: AAUUAUCCAGUUCCUUU (residues 691-707 of SEQ ID NO: 6);

target sequence 221: AACTGGATAATTTCTGTAACC (residues 2583-2603 of SEQ ID NO: 5), position in gene sequence: 2739, GC content: 33.3%, sense strand siRNA: CUGGAUAAUUUCUGUAACCUU (SEQ ID NO: 159), antisense strand siRNA: GGUUACAGAAAUUAUCCAGUU (residues 682-702 of SEQ ID NO: 6);

target sequence 222: AATTTCTGTAACCCCATCATT (residues 2591-2611 of SEQ ID NO: 5), position in gene sequence: 2747, GC content: 33.3%, sense strand siRNA: UUUCUGUAACCCCAUCAUUUU (SEQ ID NO: 160), antisense strand siRNA: AAUGAUGGGGUUACAGAAAUU (residues 674-694 of SEQ ID NO: 6);

target sequence 223: AACCCCATCATTTACAAGCCC (residues 2600-2620 of SEQ ID NO: 5), position in gene sequence: 2756, GC content: 47.6%, sense strand siRNA: CCCCAUCAUUUACAAGCCCUU (SEQ ID NO: 161), antisense strand siRNA: GGGCUUGUAAAUGAUGGGGUU (residues 665-685 of SEQ ID NO: 6);

target sequence 224: AAGCCCAAACCAAAAGCAGAA (residues 2615-2635 of SEQ ID NO: 5), position in gene sequence: 2771, GC content: 42.9%, sense strand siRNA: GCCCAAACCAAAAGCAGAAUU (SEQ ID NO: 162), antisense strand siRNA: UUCUGCUUUUGGUUUGGGCUU (residues 650-670 of SEQ ID NO: 6);

target sequence 225: AAACCAAAAGCAGAAGTTCC (residues 2621-2640 of SEQ ID NO: 5), position in gene sequence: 2777, GC content: 38.1%, sense strand siRNA: ACCAAAAGCAGAAGUUCCUU (SEQ ID NO: 163), antisense strand siRNA: GGAACUUCUGCUUUUGGUUU (residues 645-664 of SEQ ID NO: 6);

target sequence 226: AAAAGCAGAAGTTCCTG (residues 2626-2642 of SEQ ID NO: 5), position in gene sequence: 2782, GC content: 33.3%, sense strand siRNA: AAGCAGAAGUUCCUGUU (SEQ ID NO: 164), antisense strand siRNA: CAGGAACUUCUGCUUUU (residues 643-659 of SEQ ID NO: 6);

target sequence 227: AAGCAGAAGTTCCTGAA (residues 2628-2644 of SEQ ID NO: 5), position in gene sequence: 2784, GC content: 33.3%, sense strand siRNA: GCAGAAGUUCCUGAAUU (SEQ ID NO: 165), antisense strand siRNA: UUCAGGAACUUCUGCUU (residues 641-657 of SEQ ID NO: 6);

target sequence 228: AAGTTCCTGAAGACAAA (residues 2634-2650 of SEQ ID NO: 5), position in gene sequence: 2790, GC content: 28.6%, sense strand siRNA: GUUCCUGAAGACAAAUU (SEQ ID NO: 166), antisense strand siRNA: UUUGUCUUCAGGAACUU (residues 635-651 of SEQ ID NO: 6);

target sequence 229: AAGACAAACCAAAAGCTAATA (residues 2643-2663 of SEQ ID NO: 5), position in gene sequence: 2803, GC content: 28.6%, sense strand siRNA: GACAAACCAAAAGCUAAUAUU (SEQ ID NO: 167), antisense strand siRNA: UAUUAGCUUUUGGUUUGUCUU (residues 622-642 of SEQ ID NO: 6);

target sequence 230: AAACCAAAAGCTAATAGTGAA (residues 2648-2668 of SEQ ID NO: 5), position in gene sequence: 2808, GC content: 28.6%, sense strand siRNA: ACCAAAAGCUAAUAGUGAAUU (SEQ ID NO: 168), antisense strand siRNA: UUCACUAUUAGCUUUUGGUUU (residues 617-637 of SEQ ID NO: 6);

target sequence 231: AAAAGCTAATAGTGAACACAA (residues 2653-2673 of SEQ ID NO: 5), position in gene sequence: 2813, GC content: 28.6%, sense strand siRNA: AAGCUAAUAGUGAACACAAUU (SEQ ID NO: 169), antisense strand siRNA: UUGUGUUCACUAUUAGCUUUU (residues 612-632 of SEQ ID NO: 6);

target sequence 232: AAGCTAATAGTGAACACAATG (residues 2655-2675 of SEQ ID NO: 5), position in gene sequence: 2815, GC content: 33.3%, sense strand siRNA: GCUAAUAGUGAACACAAUGUU (SEQ ID NO: 170), antisense strand siRNA: CAUUGUGUUCACUAUUAGCUU (residues 610-630 of SEQ ID NO: 6);

target sequence 233: AATAGTGAACACAATGGCCCA (residues 2660-2680 of SEQ ID NO: 5), position in gene sequence: 2820, GC content: 42.9%, sense strand siRNA: UAGUGAACACAAUGGCCCAUU (SEQ ID NO: 171), antisense strand siRNA: UGGGCCAUUGUGUUCACUAUU (residues 605-625 of SEQ ID NO: 6);

target sequence 234: AACACAATGGCCCAATGGATG (residues 2667-2687 of SEQ ID NO: 5), position in gene sequence: 2827, GC content: 47.6%, sense strand siRNA: CACAAUGGCCCAAUGGAUGUU (SEQ ID NO: 172), antisense strand siRNA: CAUCCAUUGGGCCAUUGUGUU (residues 598-618 of SEQ ID NO: 6);

target sequence 235: AATGGCCCAATGGATGGACAG (residues 2672-2692 of SEQ ID NO: 5), position in gene sequence: 2832, GC content: 52.4%, sense strand siRNA: UGGCCCAAUGGAUGGACAGUU (SEQ ID NO: 173), antisense strand siRNA: CUGUCCAUCCAUUGGGCCAUU (residues 593-613 of SEQ ID NO: 6);

target sequence 236: AATGGATGGACAGAGTGGAAC (residues 2680-2700 of SEQ ID NO: 5), position in gene sequence: 2840, GC content: 47.6%, sense strand siRNA: UGGAUGGACAGAGUGGAACUU (SEQ ID NO: 174), antisense strand siRNA: GUUCCACUCUGUCCAUCCAUU (residues 585-605 of SEQ ID NO: 6);

target sequence 237: AACTGAAACTAAATCAG (residues 2698-2714 of SEQ ID NO: 5), position in gene sequence: 2858, GC content: 23.8%, sense strand siRNA: CUGAAACUAAAUCAGUU (SEQ ID NO: 175), antisense strand siRNA: CUGAUUUAGUUUCAGUU (residues 571-587 of SEQ ID NO: 6);

target sequence 238: AAACTAAATCAGATTCAACAA (residues 2703-2723 of SEQ ID NO: 5), position in gene sequence: 2867, GC content: 23.8%, sense strand siRNA: ACUAAAUCAGAUUCAACAAUU (SEQ ID NO: 176), antisense strand siRNA: UUGUUGAAUCUGAUUUAGUUU (residues 562-582 of SEQ ID NO: 6);

target sequence 239: AAATCAGATTCAACAAAAGAC (residues 2708-2728 of SEQ ID NO: 5), position in gene sequence: 2872, GC content: 28.6%, sense strand siRNA: AUCAGAUUCAACAAAAGACUU (SEQ ID NO: 177), antisense strand siRNA: GUCUUUUGUUGAAUCUGAUUU (residues 557-577 of SEQ ID NO: 6);

target sequence 240: AACAAAAGACAGCTCACAGCA (residues 2719-2739 of SEQ ID NO: 5), position in gene sequence: 2883, GC content: 42.9%, sense strand siRNA: CAAAAGACAGCUCACAGCAUU (SEQ ID NO: 178), antisense strand siRNA: UGCUGUGAGCUGUCUUUUGUU (residues 546-566 of SEQ ID NO: 6);

target sequence 241: AAAAGACAGCTCACAGCATAC (residues 2722-2742 of SEQ ID NO: 5), position in gene sequence: 2886, GC content: 42.9%, sense strand siRNA: AAGACAGCUCACAGCAUACUU (SEQ ID NO: 179), antisense strand siRNA: GUAUGCUGUGAGCUGUCUUUU543-563 of SEQ ID NO: 6);

target sequence 242: AAGACAGCTCACAGCATACTA (residues 2724-2744 of SEQ ID NO: 5), position in gene sequence: 2888, GC content: 42.9%, sense strand siRNA: GACAGCUCACAGCAUACUAUU (SEQ ID NO: 180), antisense strand siRNA: UAGUAUGCUGUGAGCUGUCUU (residues 541-561 of SEQ ID NO: 6);

target sequence 243: AAATCCTCTGGAGAGAT (residues 2744-2760 of SEQ ID NO: 5), position in gene sequence: 2908, GC content: 33.3%, sense strand siRNA: AUCCUCUGGAGAGAUUU (SEQ ID NO: 181), antisense strand siRNA: AUCUCUCCAGAGGAUUU (residues 525-541 of SEQ ID NO: 6);

target sequence 244: AAGTGGACTAAGTCTTAATTT (residues 2763-2783 of SEQ ID NO: 5), position in gene sequence: 2931, GC content: 28.6%, sense strand siRNA: GUGGACUAAGUCUUAAUUUUU (SEQ ID NO: 182), antisense strand siRNA: AAAUUAAGACUUAGUCCACUU (residues 502-522 of SEQ ID NO: 6);

target sequence 245: AAGTCTTAATTTTACCTTCAC (residues 2772-2792 of SEQ ID NO: 5), position in gene sequence: 2940, GC content: 28.6%, sense strand siRNA: GUCUUAAUUUUACCUUCACUU (SEQ ID NO: 183), antisense strand siRNA: GUGAAGGUAAAAUUAAGACUU (residues 493-513 of SEQ ID NO: 6);

target sequence 246: AATTTTACCTTCACATTAATT (residues 2779-2799 of SEQ ID NO: 5), position in gene sequence: 2947, GC content: 19%, sense strand siRNA: UUUUACCUUCACAUUAAUUUU (SEQ ID NO: 184), antisense strand siRNA: AAUUAAUGUGAAGGUAAAAUU (residues 486-506 of SEQ ID NO: 6);

target sequence 247: AATTCAAACCGTGCAAGTAAC (residues 2796-2816 of SEQ ID NO: 5), position in gene sequence: 2964, GC content: 38.1%, sense strand siRNA: UUCAAACCGUGCAAGUAACUU (SEQID NO: 185), antisense strand siRNA: GUUACUUGCACGGUUUGAAUU (residues 469-489 of SEQ ID NO: 6);

target sequence 248: AAACCGTGCAAGTAACCACG (residues 2801-2820 of SEQ ID NO: 5), position in gene sequence: 2969, GC content: 47.6%, sense strand siRNA: ACCGUGCAAGUAACCACGUU (SEQ ID NO: 186), antisense strand siRNA: CGUGGUUACUUGCACGGUUU (residues 465-484 of SEQ ID NO: 6);

target sequence 249: AAGTAACCACGGGGTCC (residues 2810-2826 of SEQ ID NO: 5), position in gene sequence: 2978, GC content: 47.6%, sense strand siRNA: GUAACCACGGGGUCCUU (SEQ ID NO: 187), antisense strand siRNA: GGACCCCGUGGUUACUU (residues 459-475 of SEQ ID NO: 6);

target sequence 250: AACCACGGGGTCCATCT (residues 2814-2830 of SEQ ID NO: 5), position in gene sequence: 2982, GC content: 47.6%, sense strand siRNA: CCACGGGGUCCAUCUUU (SEQ ID NO: 188), antisense strand siRNA: AGAUGGACCCCGUGGUU (residues 455-471 of SEQ ID NO: 6);

target sequence 251: AACAGACGCTCAGTTGTTCTT (residues 2849-2869 of SEQ ID NO: 5), position in gene sequence: 3021, GC content: 42.9%, sense strand siRNA: CAGACGCUCAGUUGUUCUUUU (SEQ ID NO: 189), antisense strand siRNA: AAGAACAACUGAGCGUCUGUU (residues 416-436 of SEQ ID NO: 6);

target sequence 252: AACCACTTTTGTCATTT (residues 2870-2886 of SEQ ID NO: 5), position in gene sequence: 3042, GC content: 23.8%, sense strand siRNA: CCACUUUUGUCAUUUUU (SEQ ID NO: 190), antisense strand siRNA: AAAUGACAAAAGUGGUU (residues 399-415 of SEQ ID NO: 6);

target sequence 253: AAAAGTGTTTTATATTGAGTG (residues 2906-2926 of SEQ ID NO: 5), position in gene sequence: 3082, GC content: 23.8%, sense strand siRNA: AAGUGUUUUAUAUUGAGUGUU (SEQ ID NO: 191), antisense strand siRNA: CACUCAAUAUAAAACACUUUU (residues 359-379 of SEQ ID NO: 6);

target sequence 254: AAGTGTTTTATATTGAGTGCA (residues 2908-2928 of SEQ ID NO: 5), position in gene sequence: 3084, GC content: 28.6%, sense strand siRNA: GUGUUUUAUAUUGAGUGCAUU (SEQ ID NO: 192), antisense strand siRNA: UGCACUCAAUAUAAAACACUU (residues 357-377 of SEQ ID NO: 6);

target sequence 255: AATTAGATTTACAAGACAATC (residues 2971-2991 of SEQ ID NO: 5), position in gene sequence: 3151, GC content: 23.8%, sense strand siRNA: UUAGAUUUACAAGACAAUCUU (SEQ ID NO: 193), antisense strand siRNA: GAUUGUCUUGUAAAUCUAAUU (residues 294-314 of SEQ ID NO: 6);

target sequence 256: AAGACAATCTAAGCTTTC (residues 2983-3000 of SEQ ID NO: 5), position in gene sequence: 3163, GC content: 28.6%, sense strand siRNA: GACAAUCUAAGCUUUCUU (SEQ ID NO: 194), antisense strand siRNA: GAAAGCUUAGAUUGUCUU (residues 285-302 of SEQ ID NO: 6);

target sequence 257: AATCTAAGCTTTCCGGA (residues 2988-3004 of SEQ ID NO: 5), position in gene sequence: 3168, GC content: 33.3%, sense strand siRNA: UCUAAGCUUUCCGGAUU (SEQ ID NO: 195), antisense strand siRNA: UCCGGAAAGCUUAGAUU (residues 281-297 of SEQ ID NO: 6);

target sequence 258: AAGCTTTCCGGATAATT (residues 2993-3009 of SEQ ID NO: 5), position in gene sequence: 3173, GC content: 28.6%, sense strand siRNA: GCUUUCCGGAUAAUUUU (SEQ ID NO: 196), antisense strand siRNA: AAUUAUCCGGAAAGCUU (residues 276-292 of SEQ ID NO: 6);

target sequence 259: AATTTTATATATCAAACATAC (residues 3006-3026 of SEQ ID NO: 5), position in gene sequence: 3190, GC content: 14.3%, sense strand siRNA: UUUUAUAUAUCAAACAUACUU (SEQ ID NO: 197), antisense strand siRNA: GUAUGUUUGAUAUAUAAAAUU (residues 259-279 of SEQ ID NO: 6);

target sequence 260: AAACATACAGGATGGATACAT (residues 3019-3039 of SEQ ID NO: 5), position in gene sequence: 3203, GC content: 33.3%, sense strand siRNA: ACAUACAGGAUGGAUACAUUU (SEQ ID NO: 198), antisense strand siRNA: AUGUAUCCAUCCUGUAUGUUU (residues 246-266 of SEQ ID NO: 6);

target sequence 261: AACAGTCTACCTTATTT (residues 3047-3063 of SEQ ID NO: 5), position in gene sequence: 3231, GC content: 23.8%, sense strand siRNA: CAGUCUACCUUAUUUUU (SEQ ID NO: 199), antisense strand siRNA: AAAUAAGGUAGACUGUU (residues 222-238 of SEQ ID NO: 6);

target sequence 262: AAAGCTTCTACTGGGATAAAC (residues 3064-3084 of SEQ ID NO: 5), position in gene sequence: 3252, GC content: 38.1%, sense strand siRNA: AGCUUCUACUGGGAUAAACUU (SEQID NO: 200), antisense strand siRNA: GUUUAUCCCAGUAGAAGCUUU (residues 201-221 of SEQ ID NO: 6);

target sequence 263: AAACCTCAATTCCTTTATTCA (residues 3081-3101 of SEQ ID NO: 5), position in gene sequence: 3269, GC content: 28.6%, sense strand siRNA: ACCUCAAUUCCUUUAUUCAUU (SEQ ID NO: 201), antisense strand siRNA: UGAAUAAAGGAAUUGAGGUUU (residues 148-204 of SEQ ID NO: 6);

target sequence 264: AATTCCTTTATTCAGGAAAGG (residues 3088-3108 of SEQ ID NO: 5), position in gene sequence: 3276, GC content: 33.3%, sense strand siRNA: UUCCUUUAUUCAGGAAAGGUU (SEQ ID NO: 202), antisense strand siRNA: CCUUUCCUGAAUAAAGGAAUU (residues 177-197 of SEQ ID NO: 6);

target sequence 265: AAAGGATACTTTATTGC (residues 3104-3120 of SEQ ID NO: 5), position in gene sequence: 3292, GC content: 23.8%, sense strand siRNA: AGGAUACUUUAUUGCUU (SEQ ID NO: 203), antisense strand siRNA: GCAAUAAAGUAUCCUUU (residues 165-181 of SEQ ID NO: 6);

target sequence 266: AAGCATAGATTTAATTGCATC (residues 3134-3154 of SEQ ID NO: 5), position in gene sequence: 3326, GC content: 28.6%, sense strand siRNA: GCAUAGAUUUAAUUGCAUCUU (SEQ ID NO: 204), antisense strand siRNA: GAUGCAAUUAAAUCUAUGCUU (residues 131-151 of SEQ ID NO: 6);

target sequence 267: AATTGCATCTTTATTTTGAAA (residues 3146-3166 of SEQ ID NO: 5), position in gene sequence: 3338, GC content: 19%, sense strand siRNA: UUGCAUCUUUAUUUUGAAAUU (SEQ ID NO: 205), antisense strand siRNA: UUUCAAAAUAAAGAUGCAAUU (residues 119-139 of SEQ ID NO: 6);

target sequence 268: AAAAACAAATGAAAATT (residues 3164-3180 of SEQ ID NO: 5), position in gene sequence: 3356, GC content: 9.5%, sense strand siRNA: AAACAAAUGAAAAUUUU (SEQ ID NO: 206), antisense strand siRNA: AAUUUUCAUUUGUUUU (residues 105-121 of SEQ ID NO: 6);

target sequence 269: AAACAAATGAAAATTGA (residues 3166-3182 of SEQ ID NO: 5), position in gene sequence: 3358, GC content: 14.3%, sense strand siRNA: ACAAAUGAAAAUUGAUU (SEQ ID NO: 207), antisense strand siRNA: UCAAUUUUCAUUUGUUU (residues 103-119 of SEQ ID NO: 6);

target sequence 270: AAATGAAAATTGATGGG (residues 3170-3186 of SEQ ID NO: 5), position in gene sequence: 3362, GC content: 23.8%, sense strand siRNA: AUGAAAAUUGAUGGGUU (SEQ ID NO: 208), antisense strand siRNA: CCCAUCAAUUUUCAUUU (residues 99-115 of SEQ ID NO: 6);

target sequence 271: AAAATTGATGGGGTTTA (residues 3175-3191 of SEQ ID NO: 5), position in gene sequence: 3367, GC content: 23.8%, sense strand siRNA: AAUUGAUGGGGUUUAUU (SEQ ID NO: 209), antisense strand siRNA: UAAACCCCAUCAAUUUU (residues 94-110 of SEQ ID NO: 6);

target sequence 272: AATTGATGGGGTTTAAA (residues 3177-3193 of SEQ ID NO: 5), position in gene sequence: 3369, GC content: 23.8%, sense strand siRNA: UUGAUGGGGUUUAAAUU (SEQ ID NO: 210), antisense strand siRNA: UUUAAACCCCAUCAAUU (residues 92-108 of SEQ ID NO: 6);

target sequence 273: AAAGCTACAGAGGCACTGACC (residues 3191-3211 of SEQ ID NO: 5), position in gene sequence: 3387, GC content: 52.4%, sense strand siRNA: AGCUACAGAGGCACUGACCUU (SEQ ID NO: 211), antisense strand siRNA: GGUCAGUGCCUCUGUAGCUUU (residues 74-94 of SEQ ID NO: 6);

target sequence 274: AATTTAAATATTAAAAC (residues 3236-3252 of SEQ ID NO: 5), position in gene sequence: 3432, GC content: 4.8%, sense strand siRNA: UUUAAAUAUUAAAACUU (SEQ ID NO: 212), antisense strand siRNA: GUUUUAAUAUUUAAAUU (residues 33-49 of SEQ ID NO: 6);

target sequence 275: AAATATTAAAACAAATAAGAG (residues 3241-3261 of SEQ ID NO: 5), position in gene sequence: 3441, GC content: 14.3%, sense strand siRNA: AUAUUAAAACAAAUAAGAGUU (SEQ ID NO: 213), antisense strand siRNA: CUCUUAUUUGUUUUAAUAUUU (residues 24-44 of SEQ ID NO: 6) of SEQ ID NO: 6);

target sequence 276: AAAACAAATAAGAGCTTTCCC (residues 3248-3268 of SEQ ID NO: 5), position in gene sequence: 3448, GC content: 33.3%, sense strand siRNA: AACAAAUAAGAGCUUUCCCUU (SEQ ID NO: 214), antisense strand siRNA: GGGAAAGCUCUUAUUUGCUUU (residues 17-37 of SEQ ID NO: 6) of SEQ ID NO: 6);

target sequence 277: AACAAATAAGAGCTTTCCCAA (residues 3250-3270 of SEQ ID NO: 5), position in gene sequence: 3450, GC content: 33.3%, sense strand siRNA: CAAAUAAGAGCUUUCCCAAUU (SEQ ID NO: 215), antisense strand siRNA: UUGGGAAAGCUCUUAUUUGUU (residues 15-35 of SEQ ID NO: 6) of SEQ ID NO: 6);

target sequence 278: AAATAAGAGCTTTCCCAAAAA (residues 3253-3273 of SEQ ID NO: 5), position in gene sequence: 3453, GC content: 28.6%, sense strand siRNA: AUAAGAGCUUUCCCAAAAAUU (SEQ ID NO: 216), antisense strand siRNA: UUUUUGGGAAAGCUCUUAUUU (residues 12-32 of SEQ ID NO: 6); and

target sequence 279: AAGAGCTTTCCCAAAAAAAAA (residues 3257-3277 of SEQ ID NO: 5), position in gene sequence: 3457, GC content: 28.6%, sense strand siRNA: GAGCUUUCCCAAAAAAAAAUU (SEQ ID NO: 217), antisense strand siRNA: UUUUUUUUUGGGAAAGCUCUU (residues 8-28 of SEQ ID NO: 6).

These target sequences, as well as any other potential Hsp110 RNAi target sequences that may be generated by other siRNA-generating algorithms/software and using other genes encoding homologous human Hsp 110 family members, without limitation, as mentioned above, are referred to herein as “Hsp 110 RNAi target sequences.” Sense and antisense Hsp110 siRNAs generated as described above, or with any other suitable algorithm/software, are referred to herein as “Hsp110 siRNAs,” which correspond to specific Hsp110 RNAi target sequences. Species-specific RNAi target sequences and siRNAs correspond to particular genes and gene products, such as Hsp110 genes and gene products, found in a given species, such as in humans.

The efficacy of each siRNA to reduce the cellular concentrations of Hsp110 can be tested in mammalian cells, e.g., human cell cultures, in vitro by western blotting for Hsp110 levels using commercially available anti-Hsp110 antiserum (StressGen) 48 hours or after another appropriate time, which can be determined depending on the cell type examined, after treatment with the desired siRNA. The antiserum cross-reacts with human and rodent Hsp110 and thus any mammalian cell line could be used. Given the partial sequence similarity between Hsp110 and Hsp70, it would be important to assay also the levels of Hsp70 for each siRNA/cell examined and ensure that the levels of this potential off-target protein are unaffected.

In one embodiment, a therapeutic regimen is provided in which Hsp110-targeted therapies are combined with ApoB-targeted therapies. The method comprises administering to a patient a therapeutic agent (for example and without limitation, siRNA(s) and/or antisense reagent(s)) targeting Hsp110 in combination with a therapeutic agent targeting ApoB. The therapeutic agents may be administered to a patient independently of the other in more than one dose and/or together in a single dose. In one embodiment, the therapeutic agents are administered to the patient together in a single dose. In another embodiment, the therapeutic agents are administered independently in alternating single doses or multiple-dose regimens. In one embodiment, the therapeutic agents are siRNA(s) and/or antisense reagent(s). As described in Crooke, R M et al., (2005), J. Lipid Res. 46:872-884, ApoB antisense reagents, including, without limitation, 5′-GTCCCTGAAGATGTCAATGC-3′ (ISIS 147764, SEQ ID NO: 286) and 5′-ATGTCAATGCCACATGTCCA-3′ (ISIS 147483, SEQ ID NO: 287), may effectively lower LDL cholesterol in a patient (see, e.g., Lemonidis, K. M. et al., “A Human Apolipoprotein B Antisense Inhibitor Reduces Aortic Sinus Plaque Volume in LDL Receptor Deficient, Human Apolipoprotein B-100 Transgenic Mice”, Arterioscler. Thromb. Vasc. Biol. 2006;26;e43-e52, “ISIS 301012, a second generation antisense oligonucleotide complementary to human apolipoprotein B-100 (apoB-100), is currently in Phase 2 trials. Phase I study results demonstrated significant and prolonged reductions in total cholesterol, LDL-C and serum apoB-100 in healthy volunteers following one month parental administration. In preclinical pharmacology studies, ISIS 301012 specifically reduced human, but not murine, hepatic apoB mRNA, protein and serum apoB-100 in human apoB-100 transgenic mice”, ISIS 301012 is described in U.S. Patent Application Publication Nos. 20040214325 and 20050009088). Likewise the ApoB RNAi reagents described in Soutschek et al., Therapeutic silencing of an endogenous gene by systemic administration of modified siRNAs, Nature 2004 Nov 11;432(7014):173-8, and evaluated in non-human primates in Zimmerman, T. S., et al. (2006), DOI:10.1038/nature04688, are useful agents for silencing, or otherwise down-regulating ApoB expression and/or protein levels in combination with the Hsp110-targeting therapies described herein. The Hsp110-targeting therapies described herein also may be combined with one or more other plasma cholesterol-lowering agents, such as, without limitation, treatment of a patient with an effective amount of a drug selected from the “statin” class of therapeutics, including, for example and without limitation, atorvastatin, fluvastatin, lovastatin, pravastatin, rosuvastatin, and simvastatin.

As used herein “yeast” is a single-celled member of the fungal families, ascomycetes, basidiomycetes and imperfect fungi that tend to be unicellular for the greater part of their life cycle. Examples of yeast genera include, without limitation, Saccharomyces, Pichia and Scizosaccharomyces. Species of suitable yeasts include, without limitation, Saccharomyces cerevisiae (S. cerevisiae) and Pichia pastoris (P. pastoris). In one embodiment, the yeast is P. pastoris, which has certain well-documented advantages, such as a native inducible promoter, AOX1, that is commercially available in a cloning system (for example and without limitation, the EasySelect™ Pichia Espression Kit, commercially available from Invitrogen Corporation of Carlsbad, Calif.). The AOX1 promoter systems, and related vectors are highly-inducible and typically are grown in Pichia strains that are designated mut^{s} (methanol utilization slow, see, for example, “EasySelect™ Pichia Espression Kit: A Manual of Methods for Expression of Recombinant Proteins using pPICZ and pPICZα in Pichia Pastoris,” Version H, Invitrogen Corporation, 1 November 2005). In one embodiment, an Hsp110-equivalent gene of a mut^{s} P. pastoris strain is mutated, as with the sse1Δ S. cerevisiae mutants described below. Sse1p rescue can be performed, as described below by inducing an Sse1p gene under control of an inducible promoter, such as the AOX1 promoter.

By “expression” it is meant the overall flow of information from a gene (without limitation, a functional genetic unit for producing a gene product, typically encoded on DNA or RNA, for some viruses, and comprising a transcriptional promoter, and other cis-acting elements, such as response elements and/or enhancers, an expressed sequence that typically encodes a protein (open-reading frame or ORF) or functional/structural RNA, and a polyadenylation sequence), to produce a gene product (typically a protein, optionally post-translationally modified or a functional/structural RNA). By “expression of genes under transcriptional control of,” or alternately “subject to control by,” a designated sequence, it is meant gene expression from a gene containing the designated sequence operably linked (functionally attached, typically in cis) to the gene. The designated sequence may be all or part of the transcriptional elements (without limitation, promoters, enhancers and response elements), and may wholly or partially regulate and/or affect transcription of a gene. A “gene for expression of” a stated gene product is a gene capable of expressing that stated gene product when placed in a suitable environment—that is, for example, when transformed, transfected of transduced into a cell, and subjected to suitable conditions for expression. In the case of a constitutive promoter “suitable conditions” means that the gene typically need only be introduced into a host cell. In the case of an inducible promoter, “suitable conditions” means when an amount of the respective inducer is administered to the expression system (e.g., cell) effective to cause expression of the gene. All nucleotide sequences described herein are provided in a 5′-to-3′ direction and all amino acid sequences described herein are provided in an N-terminal-to-C-terminal direction.

An “ApoB29 gene” is a gene encoding approximately the N-terminal 29% amino acids of ApoB 100, in one non-limiting embodiment, amino acids 1-1374 of SEQ ID NO: 2. In another non-limiting embodiment of ApoB29, as shown herein, the first 26 amino acids of amino acids 1-1374 of SEQ ID NO: 2, specifically, the “signal sequence” are replaced with the signal sequence (“pre”) and “pro” region of the yeast mating factor alpha 1 locus (“prepro,” amino acids 1-100, Kurjan J, et al., Cell. 1982 Oct;30(3):933-43) to enhance expression and targeting to the secretory pathway. This yields a construct that expresses an ApoB protein in significantly higher levels than has been seen in the past, for example in ApoB48 constructs, especially, for example and without limitation, when the yeast is propagated on glucose prior to shifting the sugar to galactose to induce the gene. This is in contrast to typical Gal induction scenarios where raffinose is commonly used to propagate cells prior to the Gal-shift. Further, expression of the ApoB29 gene is enhanced when the cells are grown first in selective media, and, then, within about 24 hours of gene induction in any assay to be performed, on non-selective media. In one embodiment, as shown in the Examples below, the shift to non-selective media is made at the same time as induction of the GAL 1,10 promoter (i.e. replacement of glucose with galactose at final concentrations of 2%). It is understood that the exact timing of removal of selective pressure and induction of the ApoB29 gene can vary, and can be altered so long as effective amounts of ApoB29 gene product can be produced.

The ApoB29 gene also may encode a polypeptide sequence, in frame with the ApoB sequences which includes an epitope or other binding partner. The purpose of this sequence is to provide a tag by which the expressed ApoB29 protein can be detected and/or purified by affinity. Without limitation, the tag may comprise a hemagglutinin epitope or another epitope, such as a poly His sequence, green fluorescent protein (GFP) or a receptor ligand.

In the examples, below, an isolated nucleic acid is provided which comprises an ApoB29 gene. A selectable marker also is provided, as well as other sequences suitable for use in vectors for use in eukaryotic and prokaryotic gene expression, including, without limitation, yeast (e.g., S. cerevisiae and P. Pastoris), E. Coli, insect and mammalian cell cloning systems. These sequences, or genetic elements, include, without limitation: promoters (inducible or non-inducible), linkers, enhancers, replication origins, operators, selectable markers, indicators (e.g., luciferase or lacZ genes and variations thereof), signal sequences, recombination sequences, etc., and selection of appropriate genetic elements depends upon the nature of the cloning system, the nucleic acid propagation system (plasmid), and the expression system, and often is dictated by which plasmids or other nucleic acids are available to the public. For example and without limitation, expression of the ApoB29 gene in yeast cells would require yeast promoters, while expression in mammalian or other eukaryotic cells, such as, without limitation: HeLa (cervix), MCF-7 (breast), HCT-116 (colon), HepG2 or Hep-3B (liver), NIH 3T3 (mouse), Rat-1, Rat-2, Chinese Hamster Ovary (CHO) cells, insect or 293 cells, would require promoters that would operate appropriately in mammalian or eukaryotic cells, such as, without limitation, CMV or SV-40 (constitutive) promoter systems or tetracycline (inducible) promoter systems.

As is shown herein, cells, such as yeast or mammalian cells can be transformed (transfected) either transiently, but preferably permanently transformed with an appropriate ApoB29 gene, and the effect of compounds on the expression of the ApoB29 gene may be determined, for example and without limitation by methods described herein. As described above, the construction of the ApoB29 gene, as well as the constituents of the nucleic acid carrying the ApoB29 gene, such as selectable markers, indicator genes etc., depends on the cloning, propagation and expression systems employed. The ApoB29 gene-containing cells are grown to a suitable cell density, and, if applicable, the ApoB29 gene is induced. The expression levels of ApoB29 gene product (mRNA or protein) can then be ascertained in the presence of a compound to be tested. The compound may be administered at any time, before, during or after initiation of expression from the ApoB29 gene. This method may be multiplexed, for example and without limitation, in a multi-well plate (for example and without limitation a 96-well dish), wherein two or more cells (cell populations) containing the ApoB29 gene are grown in two or more discrete locations (e.g., different wells) and are contacted, independently with two or more different compositions (that is cell population A is grown in well A, cell population B is grown in well B, and so on. Compound or composition A is administered to cell population A, compound or composition B is administered to cell population B and so on). Two or more discrete cell populations may be contacted with the same compound in the same concentration or in different concentrations.

In another embodiment of the compound screening method, a cell is provided which comprises a gene for expression an Hsp110 protein (an Hsp110 gene), such as, without limitation, Sse1p or a human Hsp110 protein as described herein. In this method the cell is contacted with a compound and expression of the Hsp110 protein is evaluated and a decrease in Hsp110 is indicative of a compound that would affect ApoB levels and therefore cholesterol levels.

In another embodiment, as described in further detail below, expression of an ApoB29 gene may be evaluated in cells with identifiable genetic mutations in order to ascertain the effect of that mutation of the expression of ApoB. Strains in the yeast knock-out collection, which is commercially available and is provided in a 96-well format (Yeast Knockout Strains and Yeast Magic Marker Strains, commercially available from Open Biosystems of Huntsville, Ala.), will be transformed with the ApoB29-HA expression vector and individual colonies will be selected for the presence of the vector (by growth on selective media) and will be screened for the steady-state level of ApoB-HA by colony blotting or immunoblotting cell extracts using anti-HA antiserum. Individual clones in which the levels of ApoB29-HA are elevated are likely to have a defect in the degradation of ApoB29-HA. This will be confirmed by cycloheximide chase assays (described elsewhere in the application) of the individual clone and the degradation rate will be compared to that in wt. cells. The fact that the specific gene that is knocked-out is responsible for the ApoB29-HA degradation defect can be confirmed by introducing a vector for the expression of the missing gene. If the degradation defect is rescued, then the gene contributes to ApoB29- HA degradation in yeast. If the gene has a human homologue, the effect of the corresponding gene can be studied by over-expression and RNAi, as described elsewhere herein.

In one non-limiting embodiment, a method of determining if a compound or composition affects ApoB expression and/or degradation is tested in yeast. As mentioned above, the method, in a general sense, comprises contacting a cell comprising an ApoB29 gene with the compound and determining if the compound affects expression of ApoB29. ApoB29 may be preferred to other, longer ApoB derivatives, such as, without limitation ApoB48 and ApoB100 due to its smaller size, which facilitates cellular transfection and transformation as well as correct folding within a cell and lessens non-specific effects on cell growth and viability. It was a further surprising discovery that ApoB29 underwent appropriate degradation, and thus could serve as a suitable model for Hsp110-regulated ApoB stability assays. ApoB29 has been shown to be secreted and its intracellular degradation regulated appropriately (Segrest J P, et al. J Lipid Res. 2001 Sep;42(9):1346-67). In this embodiment, the yeast cell is any appropriate yeast cell as described above, such as, without limitation, S. cerevisiae and P. pastoris. The ApoB29 gene may comprise any suitable genetic elements, such as the GAL 1,10 or AOX1 promoters.

In another surprising development, it was discovered that a portion of the firefly luciferase protein and/or a fluorescein-labeled version thereof binds to the peptide binding site of Hsp110 proteins, specifically Sse1p (See below). Thus, any compound or composition that interferes with this binding is expected to interfere with the function of Hsp110 in stabilizing ApoB. From this discovery, an assay can readily be envisioned and developed for screening compounds and compositions for their ability to reduce Hsp110 peptide-binding, which in turn would lower production and/or function of ApoB, and thus, serum cholesterol. A method of testing for whether a compound or composition affects Hsp110 peptide-binding, and consequently ApoB degradation, may be performed in any feasible manner, as a single assay, in series or in parallel. Parallel screening may be performed in a multi-well dish, such as, without limitation a 96-well dish. Samples can be individually screened by antibody binding and any suitable antibody binding detection method. Fluorescent anisotropy (fluorescent polarization), essentially as shown in FIG. 10, is fully amenable to high-throughput analysis. Reactions can be performed in a suitable multi-well plate, for example and without limitation a 96-well plate or a 384-well plate. The choice of a suitable multi-well plate or reaction chamber is a matter of experimental design choice and depends on the nature of the assay, the number of assays to be run and the equipment available to perform the assay and detect binding of the reaction constituents. Fluorescence, color changes and/or luminescence can be detected using any one of a number of plate readers commercially available, such as, without limitation, TopCount® Microplate Scintillation and Luminescence Counters (Packard Instrument Company). Reactions can be wholly or partially automated using any one of a number of automated or semi-automated robotic fluid-handling devices available commercially.

Carrier beads (particles) comprising individually addressable fluorescent dyes or quantum dots, may be employed as a suitable array. In one non-limiting example, U.S. Pat. No. 6,649,414, incorporated herein by reference for its technical disclosure, describes one such array system, which is commercially available from Luminex Corporation of Austin, Tex. under the trade name “xMAP.” In one embodiment, a first population of beads having a first fluorescent signature can be bound to a first fluorescent analyte that fluoresces (emits) at a wavelength different from any fluorescent signature of beads used in the assay. A population of beads having a second fluorescent address can be bound to a second fluorescent analyte that fluoresces at a wavelength different from any fluorescent signature of beads used in the assay. The emission spectra of each bead/analyte can be evaluated by flow cytometry, thus identifying whether and to what extent the first, second (and so on) analytes are bound to their respective bead population.

In its most generic sense, an assay for determining interference with the protein-binding domain of an Hsp110 protein will involve three constituents. The first constituent is either an Hsp110 protein or a portion thereof containing a protein binding domain (e.g. the C terminal domain, CTD). The protein binding domain of Sse1p is known, as is the protein binding domain of other Hsp110 proteins. Even if the protein binding domain of other Hsp 110 proteins is not known, the full protein can be used in this assay. In one embodiment the Hsp110 protein is isolated Sselp (GenBank Acession No. NP_{—}015219, SEQ ID NO: 3) or a portion thereof containing its C-terminal peptide binding domain (e.g., amino acid residues 375-694)). In one embodiment, the Hsp110 protein or protein binding region thereof is bound to a surface, for example the surface of an array. In another embodiment, the array is an apparatus, such as a multi-well dish, containing two or more reaction chambers. As used herein, the term “array” refers either to a set of binding reagents immobilized onto one or more substrates so that each binding reagent is at a known location or a multi-chambered apparatus containing two or more discrete, identifiable reaction chambers, such as, without limitation a 96-well dish, in which reactions comprising identified constituents are performed. In an exemplary embodiment, a set of binding reagents is immobilized onto a surface in a spatially addressable manner so that each individual binding reagent is located at a different and identifiable location on the substrate. Substrates include, without limitation, multi-well plates and beads. In one embodiment, the beads contain a marker, such as a quantum dot or fluorescent tag, so that they are individually identifiable. In one embodiment, in the context of the present disclosure, an array may be a multi-well plate containing two or more well surfaces having an Hsp110 protein or a protein binding region thereof affixed thereto.

A second constituent of the assay is a polypeptide, typically a labeled polypeptide, comprising the core amino acid sequence LICGFRVVLMYRF (SEQ ID NO: 1). The polypeptide may be the polypeptide LICGFRVVLMYRF (SEQ ID NO: 1), a larger polypeptide (without limitation, less than about 100, 50, 25 or 15 amino acids or about 13 amino acids in length) containing the core amino acid sequence LICGFRVVLMYRF (SEQ ID NO: 1), a polypeptide comprising or consisting of an amino acid sequence in which one or two of the N-terminal and/or C-terminal amino acids of LICGFRVVLMYRF (SEQ ID NO: 1) are deleted, or a portion or fragment of firefly luciferase comprising the core amino acid sequence LICGFRVVLMYRF (SEQ ID NO: 1).

The second constituent may be labeled in a manner suitable for a given assay for detection of binding of this second constituent to a first constituent. In one embodiment, the second constituent is labeled with a fluorescent reporter which permits detection of binding to the first constituent by a desired method, such as, without limitation, fluorescence anisotropy. Other labels, such as antigenic tags, binding partners (e.g., biotin) or enzymes may be used to label the second constituent, in which case, the proper binding reagent, binding partner or enzyme substrate, respectively, may be used to detect binding of the second constituent to the first.

The third constituent of the assay is the compound or composition to be tested for interference with binding of the first constituent to the second. This may be tested in a number of ways. In one non-limiting example, the first and second constituents are permitted to bind in a reaction mixture (for example and without limitation, water with appropriate amounts of buffer and salt), and afterward, the compound or composition to be tested is added to the reaction mixture and dissociation of the second constituent from the first constituent is quantified. As discussed above, dissociation can be determined by any suitable method, including fluorescence anisotropy or release of the labeled constituent from a surface-affixed constituent. Release of a bound, labeled constituent may be quantified by detection of free label in the reaction mix or by detection of label remaining bound to the substrate after contact with the compound or composition to be tested. In one embodiment, the first or second constituent contains a quenchable fluorescent label, which is quenched when bound to the second constituent and which fluoresces when released. In another embodiment, the compound or composition may be labeled in any suitable manner and its binding to the first constituent may be measured by any appropriate method.

In another embodiment, the first constituent is contacted with the compound or composition to be tested at the same time as, or before the second constituent is contacted with the first constituent. Thus, rather than testing for the ability of the compound or composition to cause dissociation of the binding of the first constituent to the second constituent, the ability of the compound or constituent to block initial binding is the focus. It may be generalized, however, that the ability of a compound to dissociate binding of one constituent to another is more desirable, but it does not necessarily mean that adding a compound or composition with or before the second constituent is added would yield an ineffective drug. The reason for this is many-fold. First, cholesterol-lowering drugs are typically administered long-term, meaning that even though a compound may not be able to drive an Hsp110 from ApoB, it would be able to prevent that association. Given that ApoB production is rapid and that the protein is continuously turned-over in the cell, and given the transient nature of the ERAD (or ERAD-like) “decision tree,” the difference between dissociation and blocking of the binding would likely be insignificant. Nevertheless, a compound or composition that has the ability to disrupt extant bindings might have a higher specific activity, possibly requiring administration of lower doses to a patient.

Although the binding assay is described above in terms of the ability of the second constituent to bind to the first, with the second being labeled and the first, where appropriate, being bound to a substrate, in many cases, the opposite also would be as effective, with the second constituent being bound to the substrate and the first reagent being labeled. The various permutations of this assay would be apparent to those of skill it the art, given this disclosure. Thus, “an assay for determining whether a compound or composition interferes with binding of the first constituent with the second constituent” means any suitable permutation or variation of the described assays.

In silico modeling methods also are available to identify compounds that could interfere with the activity of Hsp110 either with respect to its overall function or its binding to ApoB100, ApoB48, ApoB29 or the luciferase polypeptide or fluorescently-modified polypeptide comprising the amino acid sequence LICGFRVVLMYRF (SEQ ID NO: 1). Various useful in silico modeling methods are known and are available commercially.

The term “binding reagent” and like terms, refers to any compound, composition or molecule capable of specifically or substantially specifically (that is with limited cross-reactivity) binding another compound or molecule, which, in the case of immune-recognition contains an epitope. Typically, the binding reagents are antibodies, preferably monoclonal antibodies, or derivatives or analogs thereof, including without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)fragments), diabodies, tribodies or tetrabodies, which typically are covalently linked or otherwise stabilized (i.e., leucine zipper or helix stabilized) scFv fragments. “Binding reagents” also include aptamers, as are described in the art. Binding partners, such as, without limitation, biotin/avidin and receptor/substrate combinations also are considered to be within the class of “binding reagents,” though antibodies and their respective antigens also are considered to be binding partners. Further, two or more binding partners may be included in a single composition (e.g., a polypeptide chain). In one embodiment, this is a string of epitopes, such as the hemagglutinin triplet described below. In such a configuration, the epitopes contained in one compound (e.g., a polypeptide chain) do not have to be identical, as is the case with the HA triplet described herein.

The following examples are intended to illustrate specific embodiments of the present invention and are not intended to limit the scope of the inventions described herein.

### EXAMPLES

The Hsp110 Molecular Chaperone Controls ApoB Biogenesis

Using a variation on a published protocol in which the degradation of ApoB48 can be recapitulated in vitro (see, Gusarova et al., 2001, and Methods below), it was found that ApoB48 was degraded more robustly in yeast cytosol prepared from a strain lacking Sse1p, which is one isoform of the yeast Hsp110 molecular chaperone. These results suggest that Sselp aids in the stabilization of ApoB during its biogenesis, and thus that abrogating its activity destabilizes ApoB.

Methods:

Yeast Plasmid Construction:

pSLW1 was constructed using pJJB20 (see below) a plasmid with a pBM258 (Zhang, H et al. 1994 J. Biol. Chem 269 H Zhang, et al. J. Biol. Chem., Nov 1994; 269: 27799-27802) backbone, containing the yeast mating factor alpha 1 locus, amino acids 1-100 (Kuijan J, et al., Cell. 1982 Oct;30(3):933-43) inserted using the BamHI and SalI restriction sites. The plasmid also contains the GAL1,10 promoter (Johnston M, et al. Mol Cell Biol. 1984 4(8):1440-8) A triple HA (influenza hemagglutinin) tag was then PCR amplified, and the triple HA PCR product was inserted into pJJB20 by restriction digest with XbaI and SalI followed by ligation with T4 ligase to form pSLW1. The triple HA sequence includes three iterations of the single HA sequence, YPYDVPDYA (residues 1-9 of SEQ ID NO: 288), with spacer amino acids and has the sequence: YPYDVPDYAGYPYDVPDYAGSYPYDVPDYA (SEQ ID NO: 288). The nucleotide sequence encoding the triple HA polypeptide sequence terminates with two stop codons (TAA-TGA) and, for cloning, has a ClaI restriction site 5′ to the HA sequence.

Next, the gene encoding ApoB29 was amplified from the SP6-ApoB48 plasmid by PCR with the following primers: forward -5′-ATT GCC AGC ATT GCT AAA GAA GAA GGG GTA TCA CTA CTC AAG AGG AAA ATG TCA GCC TGG TCG TC-3′ (SEQ ID NO: 289) and reverse -5′-GGG ATA GCC CGC ATA CTC AGG AAC ATC GTA TGG GTA ATC GAT ACT GTA GGA GGC GGA CCA GTT GCT -3′ (SEQ ID NO: 290). The SP6-ApoB48 plasmid was constructed as follows. An SP6 driven ApoB expression vector was first described in Chuck SL, et al. (Cell. 1992 Jan 10;68(1):9-21). Into that vector, the gene encoding ApoB48 was amplified by PCR and inserted into the EcoRl sites. The resulting vector (pSPB48) permits the SP6-(in vitro) driven expression of ApoB48 and contains the ampicillin resistance gene for selection in E. coli.

The pJJB20 derivative (pSLW1), prepared as described above and containing the GAL1,10 promoter, the yeast mating factor alpha 1 locus, amino acids 1-100, and the triple HA epitope, was digested with ClaI and XbaI and both it and the ApoB29 PCR product were transformed into the common laboratory yeast strain w303 and plated on minimal media lacking uracil for selection of gap repaired pSLW1 plasmid with the ApoB29 insert. pSLW1-B29 clones were confirmed by sequence analysis.

pJJB20 was obtained from R. Fuller (University of Michigan), probably described as “pMF” in Komano H, et al. J Biol Chem. 1998 Nov 27;273(48):31648-51, and was first prepared in Zhang H, et al. 1994. pJJB20 contains the GAL1,10 sequence from pBM258 inserted into EcoR1-BamH1 sites in yCP50, and the prepro alpha factor pre-pro sequence (1-100 amino acids) was inserted into BamH1-SalI. This plasmid contains the URA3 and Ampicillin resistance markers for selection in yeast and E. coli respectively.

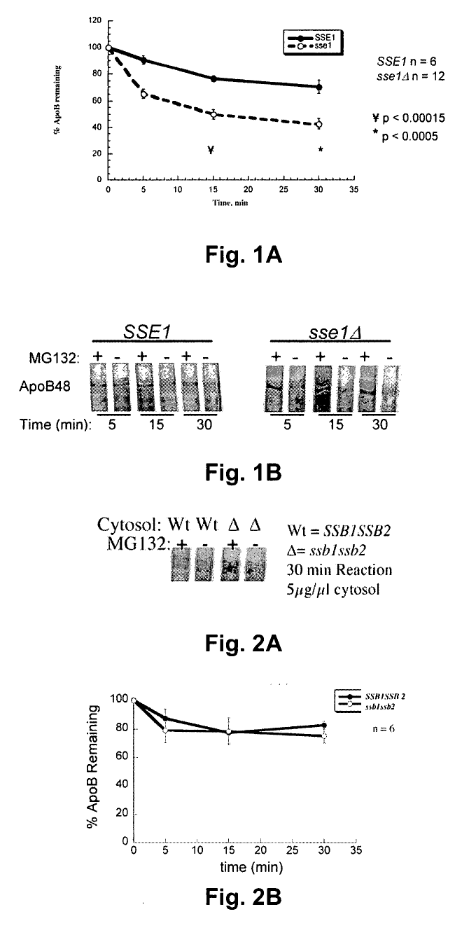

pBM258 is described in Redding K, et al. J Cell Biol. (1991)113(3):527-38. The GAL1,10 fragment is a 685-bp EcoRI-BamHI fragment containing the GALlO. GALl intergenic region (See FIG. 1 of Johnston M, et al. Mol Cell Biol. 1984 4(8): 1440-8. yCP50 can be obtained from The American Type Culture Collection (www.atcc.org, ATCC No. 37419).

In vitro Analysis of ApoB Degradation: ³⁵S methionine labeled ApoB48 was synthesized in the presence of canine pancreatic microsomes (DPM; Promega, Madison, Wis.) using the SP6-TNT Kit (Promega) and plasmid pSPB48 (see above) as previously described (Gusarova, et al., 2001). The reaction mixture was then layered onto 1 mL SH buffer (0.25M sucrose, 5 mM Hepes, pH 7.4) and centrifuged at 100,000g for 30 min at 4° C. The microsomal pellets were resuspended in 12 μl of 2×PH buffer (40 mM HEPES, pH 7.4, 220 mM KCl, 10 mM MgCl₂) and pooled. Cytosol, prepared as previously described (McCracken and Brodsky, 1996), was diluted to a specified concentration and pre-incubated with 0.25 mM MG132 (Peptides International) or an equivalent volume of DMSO for 15 min on ice. Following the pre-incubation, cytosol and ApoB48 were mixed 1:1 with these reagents at final concentrations of, 2 mM ATP, 10 mM creatine phosphate, and 100μg/mL creatine phosphokinase. Aliquots of the reaction were incubated at 37° C. for 5, 15 and 30 min or the indicated times and the reaction was quenched by adding an equal volume of 2×Urea Buffer (125 mM Tris, pH 6.8, 4% SDS, 6M Urea, 1 mM EDTA, 10 mM DTT, 250 mM β-mercaptoethanol, 20% Glycerol) and the mixture was heated at 96° C. for 4 min. The proteins were resolved on 6% SDS-polyacrylamide gels. The gels were fixed in a solution of 25% isopropanol and 10% acetic acid for 30 min, dried and exposed to a phosphorimager plate for 16h. Phosphorimager data was analyzed using Image Gauge software (Fuji Film Science Lab).

Analysis of ApoB Degradation in Yeast: Yeast transformed with pSLW1 -B29 were grown overnight at 26° C. in synthetic complete medium lacking uracil but containing glucose at a final concentration of 2% lacking uracil to logarithimic phase (OD₆₀₀=0.4-1.0). This ensured that the cells grew robustly while maintaining plasmid selection (the plasmid is URA-marked) and that the expression of ApoB29 was fully repressed. (Growth on raffinose, which is usually performed prior to galactose-inducible expression, does not fully repress synthesis from the GAL1,10 promoter and this was found to result in plasmid pSLW1-B29 loss/re-arrangement) The cells were harvested by centrifugation, washed once in water to remove the repressing sugar (i.e. glucose) and were resuspended to 0.5 OD/ml in complete media (YP) containing galactose at a final concentration of 2% at 30° C. for 5h to induce ApoB29 expression from pSLW1-B29. Although in theory plasmid selection is no longer maintained under these conditions, the use of complete medium (that is, one containing uracil and all amino acids/nutrients in abundance) was required for high-levels of ApoB29-HA expression. Protein synthesis was stopped by adding cycloheximide to a final concentration of 50 mg/ml. A total of 2 ODs of cells were removed at the indicated time-points, washed, resuspended in 1 ml water with protease inhibitors (0.25 mM MG132, 1 mM PMSF, 1 mg/mL leupeptin, 0.5 mg/ml pepstatin A) and frozen in liquid nitrogen. After samples were thawed, the total protein was precipitated (Zhang et al., 2001) and resolved on 10% SDS-Polyacrylamide gels, transferred to nitrocellulose, and probed with mouse monoclonal anti-HA antibody (Roche) to detect ApoB29 and mouse monoclonal anti-L3 antibody (J. Warner, Albert Einstein College of Medicine) that served as a loading control. Protein signals were uncovered and analyzed using enhanced chemiluminescence technology and Kodak imaging software.

Results

FIG. 1 shows ApoB degradation in vitro using wild type (SSE1) and Hsp110 mutant (sse1Δ) cytosol. Degradation is also assessed in the presence or absence of MG 132, a specific inhibitor of the proteasome, to ensure that only the proteasomal degradation of ApoB is being monitored. These results indicate enhanced degradation of ApoB48 when Hsp110 is absent.

Sse1p associated with other chaperones that bind to the ribosome. To determine whether this effect (i.e. enhanced ApoB48 degradation) was evident when any ribosome-associated molecular chaperone was disabled, the degradation of ApoB was also examined using cytosols from strains containing or lacking the Ssb molecular chaperones (Sse and Ssb chaperones have recently been shown to form a complex). However, as shown in FIGS. 2A and 2B, there was no effect on ApoB degradation regardless of whether the Ssb chaperones were present (cytosol from a strain lacking both Ssb isoforms was used), suggesting that the Sse1p effect was specific.

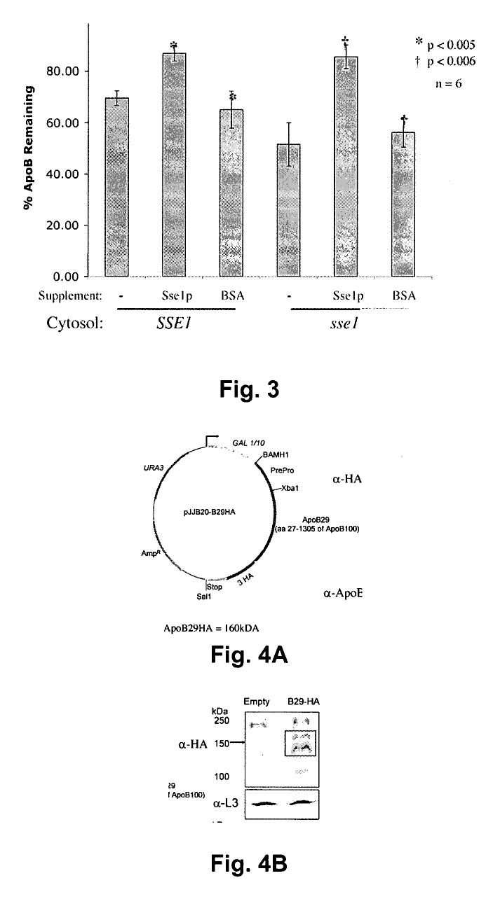

To determine whether the observed Sselp-mediated stabilization of ApoB48 was specifically due to the lack of the SSE1 protein, and not via a general or non-specific defect in the mutant cytosol, either purified Sselp or an irrelevant protein (BSA) was added into the in vitro assay in the presence of the wild type (SSE1) or delete (sse1) cytosols. Active, recombinant Sse1p, which was added into the assay as indicated, was purified as previously published (Goeckeler et al., 2002). Shown in FIG. 3 are data indicating that purified Sse1p stabilizes ApoB48 in both the mutant and wild type cytosols.

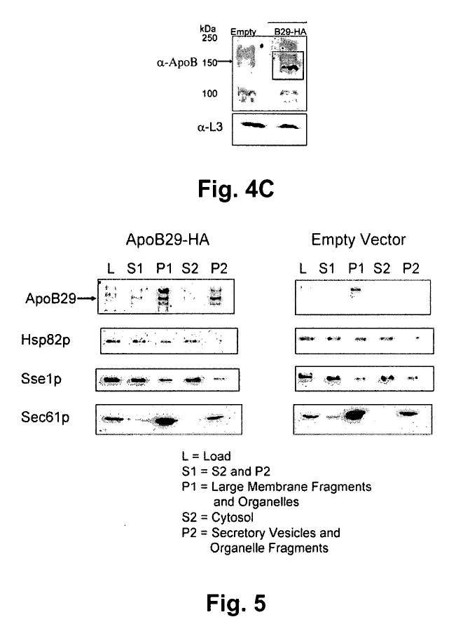

To verify these data, and potentially establish a means to screen for genes encoded in the yeast genome that might affect ApoB degradation, a yeast-expression system was established for ApoB29, an isoform that matures proficiently through the mammalian secretory pathway and whose regulated degradation mimics that of longer ApoB isoforms (See, Journal of Lipid Research 42:1346-1367, 2001). This required the construction of a new plasmid pSLW1-B29 (also known as pJJB20-B29HA) for the inducible expression of an ApoB isoform in yeast (FIG. 4A). Details on the construction of pSLW1-B29 are provided in the Methods, above. The protein, ApoB29 was expressed upon galactose-induction and contains a triple HA epitope. The expressed protein is thus observed by western blotting yeast extracts either using anti-HA (FIG. 4B) or anti-ApoB (FIG. 4C) antisera. Anti-L3 (a ribosomal protein) serves as a loading control, as shown in both of FIGS. 4B and 4C.

ApoB29, as expressed in yeast, might not insert into, and thus might not associate with organelles, but instead might remain in the cytoplasm. Preliminary evidence suggesting that this is not the case is shown in FIG. 5, in which the residence of ApoB mirrors that of a bona fide ER membrane protein, Sec61p, after cellular fractionation and western blotting the fractions using anti-HA (ApoB), anti-Hsp82p and Sse1p chaperones (largely cytoplasmic but also to a lesser degree membrane associated), and anti-Sec6 l p antiserum. This assay was also performed using yeast lacking the expression vector (“Empty Vector”).

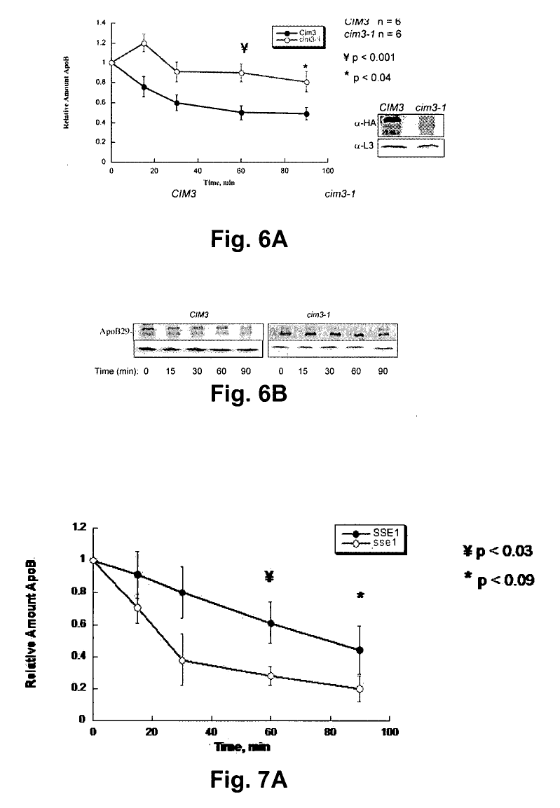

The endoplasmic reticulum-associated ApoB degradation in vitro and in mammalian cells is proteasome-dependent. To determine whether this was also the case in living yeast, ApoB29 turn-over in cells was examined with a mutated form of the proteasome “cap” (or “19S Particle”), which is required for delivery of proteasome-targeted substrates into the catalytic “core” of the proteasome. As shown in FIGS. 6A and 6B, ApoB29 degradation was slowed in the cim3-1 mutant compared to the isogenic wild type yeast (CIM3), suggesting strongly that degradation is proteasome-dependent in vivo.

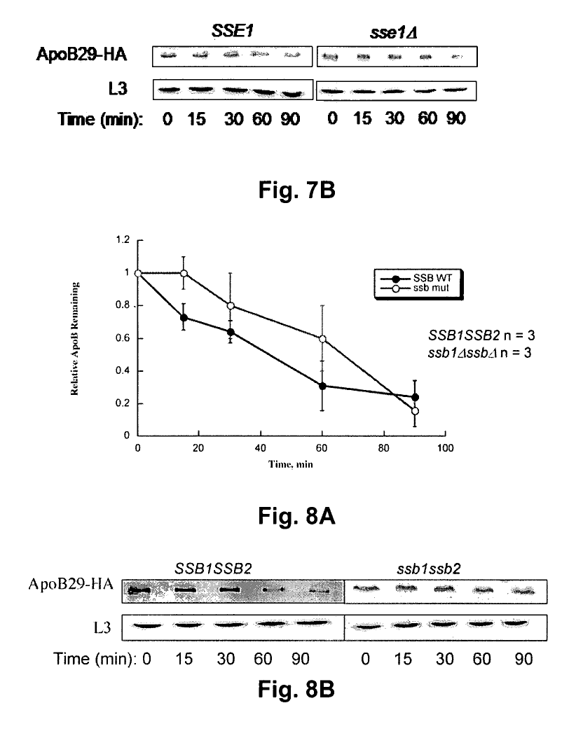

The following indicates that Sse1p also helps stabilizes ApoB in yeast, as observed in vitro. ApoB29 degradation was assessed in wild type cells (SSE1) and in yeast deleted for the SSE1 gene (sse1) and the results shown in FIGS. 7A and 7B indicate that ApoB29 is degraded more rapidly when Sse1p is absent. These data are consistent with the in vitro results, suggest again that Sselp stabilizes ApoB, and indicate that the yeast expression system can be used to assess the contributions of yeast genes on ApoB biogenesis, as described above. L3 serves as a loading control in this experiment. Degradation was assessed using a cycloheximide chase regimen that is described in the Methods above.

Because it was found that disabling the Ssb chaperones had no effect on ApoB degradation in vitro (see FIGS. 2A and 2B), these data were confirmed using the yeast expression system. Therefore, ApoB29 degradation was measured in wild type cells (SSB wt.) and in yeast lacking the Ssb chaperones (ssb1Δssb2Δ). FIGS. 8A and 8B show that there was no statistically significant difference in the extent of degradation (if anything, degradation was slightly slower when the SSBs were absent), suggesting again that the Sse1p effect is specific.

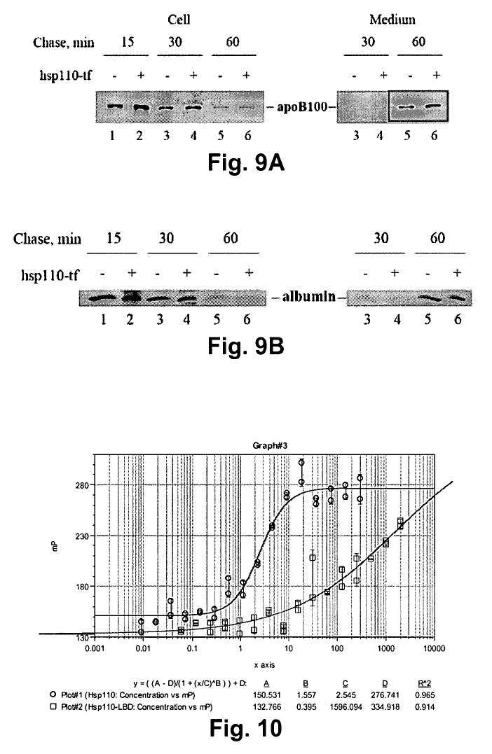

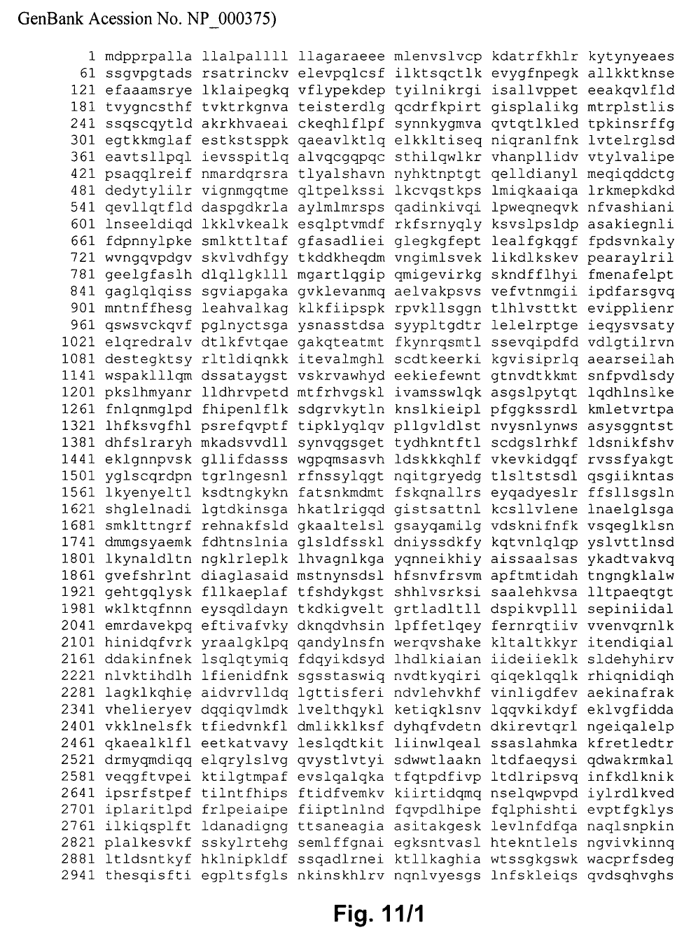

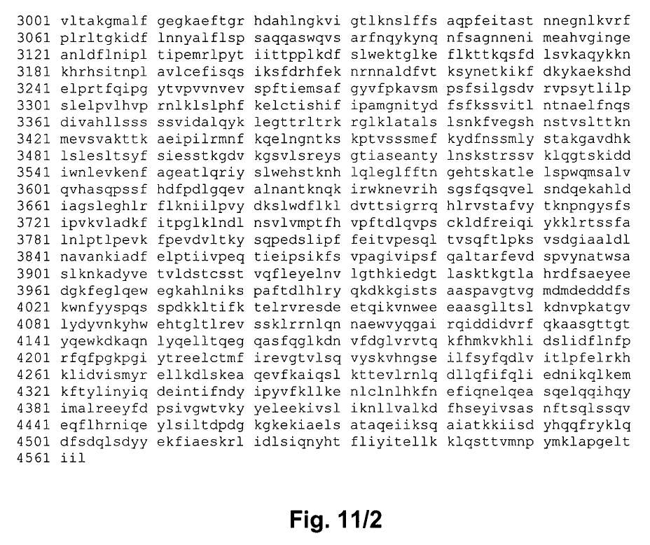

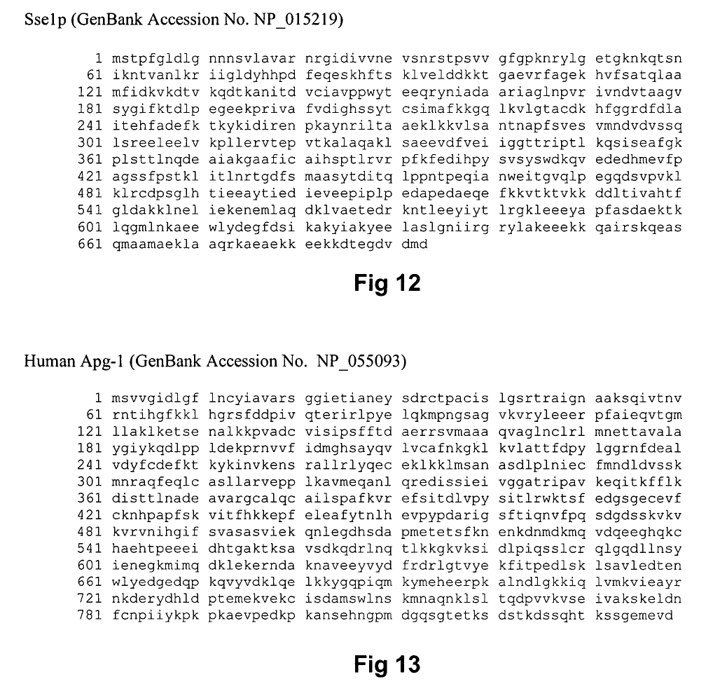

The following demonstrates that over-expression of human Hsp110 in mammalian cells affects ApoB biogenesis. An Hsp110 expression vector was obtained from Dr. John Subjeck (Roswell Park Cancer Center, N.Y.), and either that vector or a vector control was introduced into McArdle cells—a rat hepatoma cell line that also expresses full-length ApoB (that is, ApoB100). By immunoblot analysis, it was found that Hsp110 was expressed at an approximately 50% higher level when the vector contained the Hsp110-encoded insert (not shown), suggesting that the effect, if any, would be subtle. Nevertheless, it was found that the over-expression of Hsp 110 similarly led to a ˜2-fold increase in the amount of ApoB 100 secreted from these cells (FIG. 9A—box, medium, compare transfected, “tf” containing “+” or lacking “−” the Hsp110 insert). In the cell extracts (“Cell”), there was a concomitant increase in the amount of ApoB100, especially notable at the 30 min time point when Hsp110 was over-expressed (FIG. 9A). These data are consistent with Hsp110 playing a stabilizing role for ApoB100 in mammalian cells because increasing the amount of Hsp110 leads to greater ApoB secretion. These data are also consistent with the yeast data, and support the efficacy of using yeast as a means to screen for other genes that impact ApoB secretion and for small molecule modulators of ApoB biogenesis (see above). As a control, there was no effect on albumin secretion from these cells, regardless of whether Hsp110 was over-expressed (FIG. 9B). Furthermore, the over-expression of Hsp110 in the McArdle cells in the presence of a microsomal triglyceride transfer protein (MTP) inhibitor (BMS-200150; Jamil, H., D. A. Gordon, D. C. Eustice, C. M. Brooks, J. K. Dickson, Jr., Y. Chen, B. Ricci, C.-H. Chu, T. W. Harrity, C. P. Ciosek, Jr., S. A. Biller, R. E. Gregg, and J. R. Wetterau. 1996. An inhibitor of the microsomal triglyceride transfer protein inhibits apoB secretion from HepG2 cells. Proc Natl Acad Sci USA. 93, 11991-95) increased the percent of apoB recovered in the lysate and medium fractions from 14% to 28% (data not shown). These results suggest that the effect of Hsp110 on ApoB biogenesis is independent of the action of MTP. This result may also have clinical implications given that MTP inhibitors are known to reduce serum cholesterol, and therefore reducing Hsp110 production or activity (see below) in the presence of an MTP inhibitor may result in synergistic effects.

Together, these data suggest strongly that inhibiting the Sse1p homologue in mammalian cells, Hsp110, would lead to enhanced destruction of ApoB and thus reduce circulating cholesterol levels. Indeed, we have recently found that Hsp110 is expressed in the liver by western blotting hepatic cell lysates (not shown). Toward this goal, as shown in FIG. 10, a peptide was identified, that, when fluorescently labeled, binds to Sse1p (circles); the Sse1p peptide binding domain alone also binds this peptide (squares), albeit with an ˜100-fold lower affinity. In this assay, full length Sse1p and the peptide binding domain of Sse1p were bound to a 6-carboxyfluorescein-aminohexanoic acid-N-terminal-linked-polypeptide having the sequence N-LICGFRVVLMYRF-C (SEQ ID NO: 1), which was derived from firefly luciferase. Binding was assessed by fluorescence anisotropy. In this experiment, the KD for peptide binding to full-length Sse1p is —2-3 nM. Unlabeled peptide competes for binding (data not shown). These data suggest that either the peptide or a small molecule peptide mimic (that could be identified by virtue of its ability to block this peptide to bind Hsp110) might inhibit Hsp110 function, an effect that would lead to enhanced ApoB degradation and thus reduce cholesterol levels.

Having described this invention above, it will be understood to those of ordinary skill in the art that the same can be performed within a wide and equivalent range of conditions, formulations and other parameters without affecting the scope of the invention or any embodiment thereof.

We claim:

1. A method of identifying compounds that affect ApoB degradation, comprising contacting one or more cell populations in one or more discrete physical locations, in which the cells of the cell populations comprise an ApoB gene comprising an ApoB sequence, with one or more compounds and determining if the compound or compounds affect expression of the ApoB gene.

2. The method of claim 1, wherein the ApoB gene expresses an ApoB 100 or an ApoB48 protein.

3. The method of claim 1, wherein the ApoB gene is an ApoB29 gene.

4. The method of claim 3, wherein the ApoB29 gene encodes a protein comprises approximately the N-terminal 1374 amino acids of ApoB100.

5. The method of claim 3, wherein the ApoB29 gene is inducible.

6. The method of claim 5, wherein the gene is induced prior to, during or after contacting the cell with the compound.

7. The method of claim 6, wherein the cell is a yeast cell and the ApoB29 gene is galactose-inducible.

8. The method of claim 7, wherein expression of the ApoB29 gene is controlled by a GAL 1,10 promoter.

9. The method of claim 6, wherein the yeast is grown without selective pressure prior to or during induction of the inducible ApoB29 gene.

10. The method of claim 6, wherein the ApoB29 gene is encoded on DNA comprising a selectable marker.

11. The method of claim 10, wherein the cells are yeast cells, the selectable marker is a URA3 gene and the yeast cells are grown in the absence of uracil prior to or during induction of the inducible ApoB29 gene.

12. The method of claim 3, wherein the ApoB29 gene expresses a protein comprising an ApoB sequence consisting of amino acids 27-1374 of ApoB100 (SEQ ID NO: 2).

13. The method of claim 1, wherein the cell is a yeast cell.

14. The method of claim 13, wherein the ApoB29 gene encodes a yeast mating factor alpha 1 prepro sequence.

15. The method of claim 13, wherein the prepro sequence replaces the N-terminal 26 amino acids of ApoB29.

16. The method of claim 13, wherein the yeast is one of S. cerevisiae and P. pastoris.

17. The method of claim 13, wherein the yeast is grown in medium comprising glucose.

18. The method of claim 13, wherein the yeast is grown in media that substantially does not contain raffinose.

19. The method of claim 13, wherein the gene comprises an AOX1 promoter and the yeast is a mut^{s} (methanol utilization slow) Pichia pastoris.

20. The method of claim 13, wherein the yeast cell is sse1Δ.

21. The method of claim I, wherein the cell is a mammalian cell.

22. The method of claim 1, comprising contacting two or more compounds independently to two or more of the cell populations.

23. The method of claim 22, wherein the two or more cell populations are located in an array.

24. The method of claim 23, wherein the array is a multi-well dish.

25. A method of treating high serum cholesterol in a patient in which cholesterol levels are raised, comprising, down-regulating expression of an Hsp110 protein, thereby decreasing ApoB protein levels in the patient.

26. The method of claim 25, comprising administering to the patient an siRNA that corresponds to an RNAi target in an Hsp110 and which down-regulates expression of the Hsp110.

27. The method of claim 26, further comprising treating the patient with a second cholesterol-lowering therapy.

28. The method of claim 27, wherein one or both of an antisense agent and an siRNA directed to ApoB are administered to the patient.

29. The method of claim 28, wherein an antisense agent having the sequence, 5′-GTCCCTGAAGATGTCAATGC-3′ (SEQ ID NO: 286) or 5′-ATGTCAATGCCACATGTCCA-3′ (SEQ ID NO: 287) is administered to the patient.

30. The method of claim 27, wherein a statin is administered to the patient.

31. The method of claim 25, wherein the patient is a human patient.

32. The method of claim 25, comprising administering to a patient an antisense reagent for down-regulating Hsp 110 expression.

33. A cell comprising an ApoB29 gene comprising a sequence encoding an ApoB29 protein.

34. The cell of claim 33, in which the cell is a yeast cell.

35. The cell of claim 33, in which the cell is a mammalian cell.

36. The cell of claim 33, wherein the ApoB29 gene encodes a protein comprises approximately the N-terminal 1374 amino acids of ApoB100 (SEQ ID NO: 2).

37. The cell of claim 33, wherein the ApoB29 gene encodes a yeast mating factor alpha 1 prepro sequence.

38. The cell of claim 37, wherein the prepro sequence replaces the N-terminal 26 amino acids of ApoB29.

39. The cell of claim 33, wherein the ApoB29 gene expresses a protein comprising an ApoB sequence consisting of amino acids 27-1374 of ApoB100 (SEQ ID NO: 2).

40. The cell of claim 33, wherein the ApoB29 gene is inducible.

41. The cell of claim 40, wherein expression of the ApoB29 gene is controlled by a GAL 1,10 promoter.

42. The cell of claim 33, wherein the ApoB29 gene is carried on a nucleic acid containing a selectable marker.

43. The cell of claim 42, wherein the selectable marker is a URA3 gene.

44. A method of determining if a compound or composition binds an Hsp110 protein, comprising determining if the compound or composition dissociates or interferes with association of a complex of the Hsp 110 protein or a portion thereof containing a protein-binding region of the Hsp110, and an Hsp110-binding polypeptide comprising the amino acid sequence LICGFRVVLMYRF (SEQ ID NO: 1).

45. The method of claim 44, wherein the Hsp110 protein is Sse1p.

46. The method of claim 44, wherein the Hsp110 protein or a portion thereof is a portion of Sse1p containing its peptide binding region.

47. The method of claim 46, wherein the portion of Sselp consists of about residues 375-694 of SEQ ID NO: 3.

48. The method of claim 44, wherein the polypeptide is labeled.

49. The method of claim 48, wherein the polypeptide is fluorescently-labeled.

50. The method of claim 49, wherein the polypeptide is fluorescently labeled with a fluorescein compound.

51. The method of claim 50, wherein the fluorescein compound is 6-carboxyfluorescein-aminohexanoic acid.

52. The method of claim 44, wherein dissociation or interference with association is determined by fluorescent anisotropy.

53. The method of claim 44, wherein the polypeptide is a portion of firefly luciferase comprising the sequence LICGFRVVLMYRF (SEQ ID NO: 1).

54. The method of claim 44, wherein the polypeptide is less than about 100 amino acids in length.

55. The method of claim 44, wherein the polypeptide is less than about 25 amino acids in length.

56. The method of claim 44, wherein the polypeptide is about 13 amino acids in length.

57. The method of claim 44, wherein the polypeptide consists of the sequence LICGFRVVLMYRF (SEQ ID NO: 1).

58. The method of claim 57, wherein the polypeptide is fluorescently labeled.

59. The method of claim 58, wherein the polypeptide is fluorescently labeled with a fluorescein compound.

60. The method of claim 59, wherein the fluorescein compound is 6-carboxyfluorescein-aminohexanoic acid.

61. An isolated nucleic acid comprising a selectable marker useful in yeast, an ApoB29 gene sequence encoding a protein comprising, from its N-terminal to its C-terminal, a yeast prepro sequence, an ApoB29 sequence consisting of approximately amino acids 27-1374 of ApoB100 (SEQ ID NO: 2) and one or more binding partners.

62. The isolated nucleic acid of claim 61, wherein the yeast prepro sequence is a yeast mating factor alpha 1 prepro sequence.

63. The isolated nucleic acid of claim 61, wherein the ApoB29 sequence consists of amino acids 27-1374 of ApoB100 (SEQ ID NO: 2).

64. The isolated nucleic acid of claim 61, wherein one or more of the binding partners is an antigen.

65. The isolated nucleic acid of claim 64, wherein one or more of the binding partners is a sequence containing an influenza hemagglutinin epitope.

66. The isolated nucleic acid of claim 65, wherein the influenza hemagglutinin epitope of the one or more of the binding partners, independently is contained within the sequence YPYDVPDYA (residues 1-9 of SEQ ID NO: 288).

67. The isolated nucleic acid of claim 66 wherein the influenza hemagglutinin epitope of the one or more of the binding partners is contained within the sequence YPYDVPDYAGYPYDVPDYAGSYPYDVPDYA (SEQ ID NO: 288).

68. The isolated nucleic acid of claim 61, wherein the ApoB29 gene sequence comprises the sequence YPYDVPDYAGYPYDVPDYAGSYPYDVPDYA (SEQ ID NO: 288).

69. A method of identifying genes affecting ApoB expression, comprising determining the effect of a mutation in a gene in a cell on expression of an ApoB29 gene comprising a sequence encoding an ApoB29 protein.

70. The method of claim 69, in which the cell is a yeast cell.

71. The method of claim 69, in which the cell is a mammalian cell.

72. The method of claim 69, wherein the ApoB29 gene encodes a protein comprises approximately the N-terminal 1374 amino acids of ApoB100 (SEQ ID NO: 2).

73. The method of claim 69, wherein the ApoB29 gene encodes a yeast mating factor alpha 1 prepro sequence.

74. The method of claim 73, wherein the prepro sequence replaces the N-terminal 26 amino acids of ApoB29.

75. The method of claim 69, wherein the ApoB29 gene expresses a protein comprising an ApoB sequence consisting of amino acids 27-1374 of ApoB100 (SEQ ID NO: 2).

76. The method of claim 69, wherein the ApoB29 gene is inducible.

77. The method of claim 76, wherein the ApoB29 gene is galactose-inducible.

78. The method of claim 77, wherein expression of the ApoB29 gene is controlled by a GAL 1,10 promoter.

79. The method of claim 69, wherein the ApoB29 gene is carried on a nucleic acid containing a selectable marker.

80. The method of claim 79, wherein the selectable marker is a URA3 gene.

81. A method of identifying compounds that affect ApoB degradation, comprising contacting one or more cell populations in one or more discrete physical locations, in which the cells of the cell populations comprise an Hsp110 gene comprising an Hsp110 sequence, with one or more compounds and determining if the compound or compounds affect steady-state levels of the Hsp110 protein.

82. The method of claim 81, wherein the Hsp110 gene encodes and Sselp protein.

83. The method of claim 81, wherein the Hsp110 gene encodes a human Hsp110 protein.

84. The method of claim 81, the cell further comprising and ApoB29 gene.

85. A method of identifying a compound that interferes with Hsp110 ATPase function comprising using in silico modeling to identify a compound that interferes with Hsp110 ATPase function or binding to one of ApoB100, ApoB48, ApoB29 or a polypeptide or fluorescently-labeled polypeptide comprising the amino acid sequence LICGFRVVLMYRF (SEQ ID NO: 1).


SEQUENCE LISTING (290 sequences)

SEQ ID NO: 1 - protein, 13 aa, Photinus pyralis
LICGFRVVLMYRF

SEQ ID NO: 2 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 3 - protein, 693 aa, Saccharomyces cerevisiae
MSTPFGLDLGNNNSVLAVARNRGIDIVVNEVSNRSTPSVVGFGPKNRYLGETGKNKQTSNIKNTVANLKRIIGLDYHHPDFEQESKHFTSKLVELDDKKTGAEVRFAGEKHVFSATQLAAMFIDKVKDTVKQDTKANITDVCIAVPPWYTEEQRYNIADAARIAGLNPVRIVNDVTAAGVSYGIFKTDLPEGEEKPRIVAFVDIGHSSYTCSIMAFKKGQLKVLGTACDKHFGGRDFDLAITEHFADEFKTKYKIDIRENPKAYNRILTAAEKLKKVLSANTNAPFSVESVMNDVDVSSQLSREELEELVKPLLERVTEPVTKALAQAKLSAEEVDFVEIIGGTTRIPTLKQSISEAFGKPLSTTLNQDEAIAKGAAFICAIHSPTLRVRPFKFEDIHPYSVSYSWDKQVEDEDHMEVFPAGSSFPSTKLITLNRTGDFSMAASYTDITQLPPNTPEQIANWEITGVQLPEGQDSVPVKLKLRCDPSGLHTIEEAYTIEDIEVEEPIPLPEDAPEDAEQEFKKVTKTVKKDDLTIVAHTFGLDAKKLNELIEKENEMLAQDKLVAETEDRKNTLEEYIYTLRGKLEEEYAPFASDAEKTKLQGMLNKAEEWLYDEGFDSIKAKYIAKYEELASLGNIIRGRYLAKEEEKKQAIRSKQEASQMAAMAEKLAAQRKAEAEKKEEKKDTEGDVDMD

SEQ ID NO: 4 - protein, 839 aa, Homo sapiens
MSVVGIDLGFLNCYIAVARSGGIETIANEYSDRCTPACISLGSRTRAIGNAAKSQIVTNVRNTIHGFKKLHGRSFDDPIVQTERIRLPYELQKMPNGSAGVKVRYLEEERPFAIEQVTGMLLAKLKETSENALKKPVADCVISIPSFFTDAERRSVMAAAQVAGLNCLRLMNETTAVALAYGIYKQDLPPLDEKPRNVVFIDMGHSAYQVLVCAFNKGKLKVLATTFDPYLGGRNFDEALVDYFCDEFKTKYKINVKENSRALLRLYQECEKLKKLMSANASDLPLNIECFMNDLDVSSKMNRAQFEQLCASLLARVEPPLKAVMEQANLQREDISSIEIVGGATRIPAVKEQITKFFLKDISTTLNADEAVARGCALQCAILSPAFKVREFSITDLVPYSITLRWKTSFEDGSGECEVFCKNHPAPFSKVITFHKKEPFELEAFYTNLHEVPYPDARIGSFTIQNVFPQSDGDSSKVKVKVRVNIHGIFSVASASVIEKQNLEGDHSDAPMETETSFKNENKDNMDKMQVDQEEGHQKCHAEHTPEEEIDHTGAKTKSAVSDKQDRLNQTLKKGKVKSIDLPIQSSLCRQLGQDLLNSYIENEGKMIMQDKLEKERNDAKNAVEEYVYDFRDRLGTVYEKFITPEDLSKLSAVLEDTENWLYEDGEDQPKQVYVDKLQELKKYGQPIQMKYMEHEERPKALNDLGKKIQLVMKVIEAYRNKDERYDHLDPTEMEKVEKCISDAMSWLNSKMNAQNKLSLTQDPVVKVSEIVAKSKELDNFCNPIIYKPKPKAEVPEDKPKANSEHNGPMDGQSGTETKSDSTKDSSQHTKSSGEMEVD

SEQ ID NO: 5 - DNA, 3284 bp, Homo sapiens
gccggttggaggcggccgaaccgcagtagggaaagacccaggctgcgggacgcggtgcaggctgcggcgctgacggcctctgctccttccgcgggtttccgactccctgccctagattttctgcttagcgacttggggtcccctctcgtttgcttctggtaggagtcgcaatcccagcagcaatagcccagaagaggacacggttcccgtaccgaagggttcagtaccagcagcccgaccatcacgcggcgggatgtctgtggttggcattgacctcggctttctcaactgctacattgctgtcgcgagaagtggcggcatcgagaccatcgccaatgagtacagcgacaggtgtaccccggcctgtatatcattgggatcaagaactcgagccattggaaatgcagcaaagagccagatagtcacgaacgtaagaaatacaattcatggcttcaaaaagcttcatgggcgatcatttgatgatcccattgtgcaaactgaaaggatcaggcttccctatgaactgcagaaaatgcctaatggaagtgcaggagttaaggtgcggtacttagaggaagagagaccttttgcaattgagcaagttactggaatgctgttagccaagcttaaagagacttcagaaaatgctttgaagaaaccagtggctgactgtgtgatttcaattcctagcttttttactgatgccgagagaagatctgtgatggctgcagcccaggttgcaggcttaaattgtttaaggttgatgaatgaaactactgcagttgcactggcgtatggaatttataaacaggatcttcccccattagatgagaaaccaagaaatgtagtatttattgatatgggacattctgcctatcaggtcttggtttgtgcttttaacaaaggaaaacttaaagtcttggctactacctttgatccatatttgggtggcaggaactttgatgaggctttagtagactacttctgtgatgagttcaagaccaaatataagataaatgtgaaagaaaactctcgggccttgttgcgtttatatcaggaatgtgaaaaactaaagaagctaatgagtgcaaatgcatcagatcttccattgaacattgagtgtttcatgaatgaccttgatgtttctagtaaaatgaacagggctcaatttgaacaactgtgtgcttcccttttggccagggttgaaccacctttaaaagcagtaatggaacaagctaacttacaacgtgaagacattagtagtatagaaattgtaggaggagcaacacgaattcctgcagtgaaagaacaaatcactaaattctttcttaaagacataagtaccacattaaatgctgatgaagctgttgcaagaggatgtgcgttacagtgtgcgattctctcaccagcatttaaagtgcgtgaattttccataacagaccttgttccctattcaatcacattaaggtggaagacctcttttgaagatggaagtggggaatgtgaagttttctgtaagaaccatcctgccccattctcaaaagtcattactttccacaagaaggaaccatttgaactagaagcattttatactaatttacatgaagtgccttatcctgatgcaagaattgggagcttcactattcagaatgtttttccacagtctgatggtgatagttccaaagtgaaggttaaagttcgtgttaacatccatggaatcttcagtgtggctagcgcatcagtaattgagaagcaaaatttggaaggcgatcacagtgatgctccaatggagacagaaacttcatttaaaaatgaaaacaaagataatatggataaaatgcaggttgatcaagaagaagggcatcaaaaatgtcatgctgaacacactccagaagaggaaattgatcatacaggagccaaaacaaagtcagctgtctcagacaaacaagaccgattaaatcagacacttaaaaaaggaaaagtcaaaagtattgatctaccgatccagagtagcctatgtagacaactaggccaagatcttctcaacagctacattgaaaatgaggggaagatgatcatgcaagataagttagagaaagaaagaaatgatgctaagaatgccgttgaagaatatgtatatgattttagagacaggctgggcactgtctatgaaaaattcatcactccagaagacttgagtaaactgtctgcagtattggaagacacagaaaattggctttatgaagacggagaggaccaacctaaacaagtttatgtggataagcttcaagaactaaagaaatacggccagcctattcaaatgaagtacatggagcatgaagagagaccaaaagccttaaatgacttgggaaaaaagatccaacttgtcatgaaagtgatagaagcttatagaaacaaggatgaaagatatgatcatctggatcctactgaaatggaaaaggttgaaaaatgtatcagtgatgccatgagttggctgaatagtaagatgaatgcacagaacaaactaagtctcactcaagatcctgtggtaaaagtttcagaaatagtagcaaagtcaaaggaactggataatttctgtaaccccatcatttacaagcccaaaccaaaagcagaagttcctgaagacaaaccaaaagctaatagtgaacacaatggcccaatggatggacagagtggaactgaaactaaatcagattcaacaaaagacagctcacagcatactaaatcctctggagagatggaagtggactaagtcttaattttaccttcacattaattcaaaccgtgcaagtaaccacggggtccatcttttacatctggtacacacaacagacgctcagttgttcttaaccacttttgtcatttgttttttggagtagttttgaaaagtgttttatattgagtgcacttctgttcatttccattgctgcttatatgcagtgttagccgaattagatttacaagacaatctaagctttccggataattttatatatcaaacatacaggatggatacatagttggcaacagtctaccttatttaaagcttctactgggataaacctcaattcctttattcaggaaaggatactttattgcattattgttgcagaagcatagatttaattgcatctttattttgaaaaacaaatgaaaattgatggggtttaaagctacagaggcactgacctttttctagttattgtattgctacaatttaaatattaaaacaaataagagctttcccaaaaaaaaaaaaaaaa

SEQ ID NO: 6 - RNA, 3284 nt, Homo sapiens
uuuuuuuuuuuuuuuugggaaagcucuuauuuguuuuaauauuuaaauuguagcaauacaauaacuagaaaaaggucagugccucuguagcuuuaaaccccaucaauuuucauuuguuuuucaaaauaaagaugcaauuaaaucuaugcuucugcaacaauaaugcaauaaaguauccuuuccugaauaaaggaauugagguuuaucccaguagaagcuuuaaauaagguagacuguugccaacuauguauccauccuguauguuugauauauaaaauuauccggaaagcuuagauugucuuguaaaucuaauucggcuaacacugcauauaagcagcaauggaaaugaacagaagugcacucaauauaaaacacuuuucaaaacuacuccaaaaaacaaaugacaaaagugguuaagaacaacugagcgucuguuguguguaccagauguaaaagauggaccccgugguuacuugcacgguuugaauuaaugugaagguaaaauuaagacuuaguccacuuccaucucuccagaggauuuaguaugcugugagcugucuuuuguugaaucugauuuaguuucaguuccacucuguccauccauugggccauuguguucacuauuagcuuuugguuugucuucaggaacuucugcuuuugguuugggcuuguaaaugaugggguuacagaaauuauccaguuccuuugacuuugcuacuauuucugaaacuuuuaccacaggaucuugagugagacuuaguuuguucugugcauucaucuuacuauucagccaacucauggcaucacugauacauuuuucaaccuuuuccauuucaguaggauccagaugaucauaucuuucauccuuguuucuauaagcuucuaucacuuucaugacaaguuggaucuuuuuucccaagucauuuaaggcuuuuggucucucuucaugcuccauguacuucauuugaauaggcuggccguauuucuuuaguucuugaagcuuauccacauaaacuuguuuagguugguccucuccgucuucauaaagccaauuuucugugucuuccaauacugcagacaguuuacucaagucuucuggagugaugaauuuuucauagacagugcccagccugucucuaaaaucauauacauauucuucaacggcauucuuagcaucauuucuuucuuucucuaacuuaucuugcaugaucaucuuccccucauuuucaauguagcuguugagaagaucuuggccuaguugucuacauaggcuacucuggaucgguagaucaauacuuuugacuuuuccuuuuuuaagugucugauuuaaucggucuuguuugucugagacagcugacuuuguuuuggcuccuguaugaucaauuuccucuucuggaguguguucagcaugacauuuuugaugcccuucuucuugaucaaccugcauuuuauccauauuaucuuuguuuucauuuuuaaaugaaguuucugucuccauuggagcaucacugugaucgccuuccaaauuuugcuucucaauuacugaugcgcuagccacacugaagauuccauggauguuaacacgaacuuuaaccuucacuuuggaacuaucaccaucagacuguggaaaaacauucugaauagugaagcucccaauucuugcaucaggauaaggcacuucauguaaauuaguauaaaaugcuucuaguucaaaugguuccuucuuguggaaaguaaugacuuuugagaauggggcaggaugguucuuacagaaaacuucacauuccccacuuccaucuucaaaagaggucuuccaccuuaaugugauugaauagggaacaaggucuguuauggaaaauucacgcacuuuaaaugcuggugagagaaucgcacacuguaacgcacauccucuugcaacagcuucaucagcauuuaaugugguacuuaugucuuuaagaaagaauuuagugauuuguucuuucacugcaggaauucguguugcuccuccuacaauuucuauacuacuaaugucuucacguuguaaguuagcuuguuccauuacugcuuuuaaaggugguucaacccuggccaaaagggaagcacacaguuguucaaauugagcccuguucauuuuacuagaaacaucaaggucauucaugaaacacucaauguucaauggaagaucugaugcauuugcacucauuagcuucuuuaguuuuucacauuccugauauaaacgcaacaaggcccgagaguuuucuuucacauuuaucuuauauuuggucuugaacucaucacagaaguagucuacuaaagccucaucaaaguuccugccacccaaauauggaucaaagguaguagccaagacuuuaaguuuuccuuuguuaaaagcacaaaccaagaccugauaggcagaaugucccauaucaauaaauacuacauuucuugguuucucaucuaaugggggaagauccuguuuauaaauuccauacgccagugcaacugcaguaguuucauucaucaaccuuaaacaauuuaagccugcaaccugggcugcagccaucacagaucuucucucggcaucaguaaaaaagcuaggaauugaaaucacacagucagccacugguuucuucaaagcauuuucugaagucucuuuaagcuuggcuaacagcauuccaguaacuugcucaauugcaaaaggucucucuuccucuaaguaccgcaccuuaacuccugcacuuccauuaggcauuuucugcaguucauagggaagccugauccuuucaguuugcacaaugggaucaucaaaugaucgcccaugaagcuuuuugaagccaugaauuguauuucuuacguucgugacuaucuggcucuuugcugcauuuccaauggcucgaguucuugaucccaaugauauacaggccgggguacaccugucgcuguacucauuggcgauggucucgaugccgccacuucucgcgacagcaauguagcaguugagaaagccgaggucaaugccaaccacagacaucccgccgcgugauggucgggcugcugguacugaacccuucgguacgggaaccguguccucuucugggcuauugcugcugggauugcgacuccuaccagaagcaaacgagaggggaccccaagucgcuaagcagaaaaucuagggcagggagucggaaacccgcggaaggagcagaggccgucagcgccgcagccugcaccgcgucccgcagccugggucuuucccuacugcgguucggccgccuccaaccggc

SEQ ID NO: 7 - RNA, 21 nt, Homo sapiens
ccgcaguagggaaagacccuu

SEQ ID NO: 8 - RNA, 21 nt, Homo sapiens
agacccaggcugcgggacguu

SEQ ID NO: 9 - RNA, 18 nt, Homo sapiens
ucccagcagcaauagcuu

SEQ ID NO: 10 - RNA, 21 nt, Homo sapiens
uagcccagaagaggacacguu

SEQ ID NO: 11 - RNA, 21 nt, Homo sapiens
gaggacacgguucccguacuu

SEQ ID NO: 12 - RNA, 21 nt, Homo sapiens
ggguucaguaccagcagccuu

SEQ ID NO: 13 - RNA, 18 nt, Homo sapiens
cugcuacauugcugucuu

SEQ ID NO: 14 - RNA, 21 nt, Homo sapiens
guggcggcaucgagaccauuu

SEQ ID NO: 15 - RNA, 21 nt, Homo sapiens
ugaguacagcgacagguguuu

SEQ ID NO: 16 - RNA, 21 nt, Homo sapiens
gaacucgagccauuggaaauu

SEQ ID NO: 17 - RNA, 21 nt, Homo sapiens
cucgagccauuggaaaugcuu

SEQ ID NO: 18 - RNA, 21 nt, Homo sapiens
augcagcaaagagccagauuu

SEQ ID NO: 19 - RNA, 18 nt, Homo sapiens
agagccagauagucacuu

SEQ ID NO: 20 - RNA, 21 nt, Homo sapiens
cguaagaaauacaauucauuu

SEQ ID NO: 21 - RNA, 21 nt, Homo sapiens
gaaauacaauucauggcuuuu

SEQ ID NO: 22 - RNA, 21 nt, Homo sapiens
auacaauucauggcuucaauu

SEQ ID NO: 23 - RNA, 21 nt, Homo sapiens
uucauggcuucaaaaagcuuu

SEQ ID NO: 24 - RNA, 21 nt, Homo sapiens
aaagcuucaugggcgaucauu

SEQ ID NO: 25 - RNA, 21 nt, Homo sapiens
agcuucaugggcgaucauuuu

SEQ ID NO: 26 - RNA, 21 nt, Homo sapiens
acugaaaggaucaggcuucuu

SEQ ID NO: 27 - RNA, 21 nt, Homo sapiens
aggaucaggcuucccuauguu

SEQ ID NO: 28 - RNA, 19 nt, Homo sapiens
cugcagaaaaugccuaauu

SEQ ID NO: 29 - RNA, 18 nt, Homo sapiens
aaugccuaauggaaguuu

SEQ ID NO: 30 - RNA, 18 nt, Homo sapiens
ugccuaauggaagugcuu

SEQ ID NO: 31 - RNA, 18 nt, Homo sapiens
uggaagugcaggaguuuu

SEQ ID NO: 32 - RNA, 21 nt, Homo sapiens
gugcaggaguuaaggugcguu

SEQ ID NO: 33 - RNA, 21 nt, Homo sapiens
ggugcgguacuuagaggaauu

SEQ ID NO: 34 - RNA, 21 nt, Homo sapiens
gagagaccuuuugcaauuguu

SEQ ID NO: 35 - RNA, 18 nt, Homo sapiens
uugagcaaguuacugguu

SEQ ID NO: 36 - RNA, 21 nt, Homo sapiens
ugcuguuagccaagcuuaauu

SEQ ID NO: 37 - RNA, 21 nt, Homo sapiens
gcuuaaagagacuucagaauu

SEQ ID NO: 38 - RNA, 21 nt, Homo sapiens
agagacuucagaaaaugcuuu

SEQ ID NO: 39 - RNA, 19 nt, Homo sapiens
aaugcuuugaagaaaccuu

SEQ ID NO: 40 - RNA, 18 nt, Homo sapiens
ugcuuugaagaaaccauu

SEQ ID NO: 41 - RNA, 18 nt, Homo sapiens
gaaaccaguggcugacuu

SEQ ID NO: 42 - RNA, 18 nt, Homo sapiens
accaguggcugacuguuu

SEQ ID NO: 43 - RNA, 21 nt, Homo sapiens
uuccuagcuuuuuuacugauu

SEQ ID NO: 44 - RNA, 18 nt, Homo sapiens
gaucugugauggcugcuu

SEQ ID NO: 45 - RNA, 21 nt, Homo sapiens
auuguuuaagguugaugaauu

SEQ ID NO: 46 - RNA, 21 nt, Homo sapiens
gguugaugaaugaaacuacuu

SEQ ID NO: 47 - RNA, 18 nt, Homo sapiens
ugaaacuacugcaguuuu

SEQ ID NO: 48 - RNA, 18 nt, Homo sapiens
acuacugcaguugcacuu

SEQ ID NO: 49 - RNA, 21 nt, Homo sapiens
uuuauaaacaggaucuuccuu

SEQ ID NO: 50 - RNA, 21 nt, Homo sapiens
acaggaucuucccccauuauu

SEQ ID NO: 51 - RNA, 18 nt, Homo sapiens
accaagaaauguaguauu

SEQ ID NO: 52 - RNA, 18 nt, Homo sapiens
gaaauguaguauuuauuu

SEQ ID NO: 53 - RNA, 21 nt, Homo sapiens
auguaguauuuauugauauuu

SEQ ID NO: 54 - RNA, 18 nt, Homo sapiens
caaaggaaaacuuaaauu

SEQ ID NO: 55 - RNA, 21 nt, Homo sapiens
aggaaaacuuaaagucuuguu

SEQ ID NO: 56 - RNA, 21 nt, Homo sapiens
aacuuaaagucuuggcuacuu

SEQ ID NO: 57 - RNA, 21 nt, Homo sapiens
cuuaaagucuuggcuacuauu

SEQ ID NO: 58 - RNA, 21 nt, Homo sapiens
agucuuggcuacuaccuuuuu

SEQ ID NO: 59 - RNA, 18 nt, Homo sapiens
cuuugaugaggcuuuauu

SEQ ID NO: 60 - RNA, 21 nt, Homo sapiens
gaccaaauauaagauaaauuu

SEQ ID NO: 61 - RNA, 17 nt, Homo sapiens
auauaagauaaauguuu

SEQ ID NO: 62 - RNA, 17 nt, Homo sapiens
gauaaaugugaaagauu

SEQ ID NO: 63 - RNA, 17 nt, Homo sapiens
augugaaagaaaacuuu

SEQ ID NO: 64 - RNA, 21 nt, Homo sapiens
agaaaacucucgggccuuguu

SEQ ID NO: 65 - RNA, 21 nt, Homo sapiens
aacucucgggccuuguugcuu

SEQ ID NO: 66 - RNA, 21 nt, Homo sapiens
cucucgggccuuguugcguuu

SEQ ID NO: 67 - RNA, 21 nt, Homo sapiens
ugugaaaaacuaaagaagcuu

SEQ ID NO: 68 - RNA, 17 nt, Homo sapiens
aaacuaaagaagcuauu

SEQ ID NO: 69 - RNA, 17 nt, Homo sapiens
acuaaagaagcuaauuu

SEQ ID NO: 70 - RNA, 17 nt, Homo sapiens
agaagcuaaugaguguu

SEQ ID NO: 71 - RNA, 17 nt, Homo sapiens
gcuaaugagugcaaauu

SEQ ID NO: 72 - RNA, 21 nt, Homo sapiens
ugagugcaaaugcaucagauu

SEQ ID NO: 73 - RNA, 21 nt, Homo sapiens
augcaucagaucuuccauuuu

SEQ ID NO: 74 - RNA, 21 nt, Homo sapiens
cauugaguguuucaugaauuu

SEQ ID NO: 75 - RNA, 17 nt, Homo sapiens
ugaccuugauguuucuu

SEQ ID NO: 76 - RNA, 21 nt, Homo sapiens
aaugaacagggcucaauuuuu

SEQ ID NO: 77 - RNA, 21 nt, Homo sapiens
ugaacagggcucaauuugauu

SEQ ID NO: 78 - RNA, 21 nt, Homo sapiens
cagggcucaauuugaacaauu

SEQ ID NO: 79 - RNA, 21 nt, Homo sapiens
uuugaacaacugugugcuuuu

SEQ ID NO: 80 - RNA, 21 nt, Homo sapiens
caacugugugcuucccuuuuu

SEQ ID NO: 81 - RNA, 21 nt, Homo sapiens
cugugugcuucccuuuugguu

SEQ ID NO: 82 - RNA, 21 nt, Homo sapiens
ccaccuuuaaaagcaguaauu

SEQ ID NO: 83 - RNA, 21 nt, Homo sapiens
aagcaguaauggaacaagcuu

SEQ ID NO: 84 - RNA, 21 nt, Homo sapiens
gcaguaauggaacaagcuauu

SEQ ID NO: 85 - RNA, 21 nt, Homo sapiens
uggaacaagcuaacuuacauu

SEQ ID NO: 86 - RNA, 21 nt, Homo sapiens
caagcuaacuuacaacguguu

SEQ ID NO: 87 - RNA, 21 nt, Homo sapiens
gcuaacuuacaacgugaaguu

SEQ ID NO: 88 - RNA, 21 nt, Homo sapiens
cuuacaacgugaagacauuuu

SEQ ID NO: 89 - RNA, 17 nt, Homo sapiens
cgugaagacauuaguuu

SEQ ID NO: 90 - RNA, 17 nt, Homo sapiens
gacauuaguaguauauu

SEQ ID NO: 91 - RNA, 21 nt, Homo sapiens
auuguaggaggagcaacacuu

SEQ ID NO: 92 - RNA, 21 nt, Homo sapiens
cacgaauuccugcagugaauu

SEQ ID NO: 93 - RNA, 21 nt, Homo sapiens
uuccugcagugaaagaacauu

SEQ ID NO: 94 - RNA, 17 nt, Homo sapiens
agaacaaaucacuaauu

SEQ ID NO: 95 - RNA, 17 nt, Homo sapiens
caaaucacuaaauucuu

SEQ ID NO: 96 - RNA, 17 nt, Homo sapiens
aucacuaaauucuuuuu

SEQ ID NO: 97 - RNA, 17 nt, Homo sapiens
auucuuucuuaaagauu

SEQ ID NO: 98 - RNA, 21 nt, Homo sapiens
agacauaaguaccacauuauu

SEQ ID NO: 99 - RNA, 21 nt, Homo sapiens
guaccacauuaaaugcugauu

SEQ ID NO: 100 - RNA, 21 nt, Homo sapiens
augcugaugaagcuguugcuu

SEQ ID NO: 101 - RNA, 19 nt, Homo sapiens
gcuguugcaagaggauguu

SEQ ID NO: 102 - RNA, 17 nt, Homo sapiens
gaggaugugcguuacuu

SEQ ID NO: 103 - RNA, 21 nt, Homo sapiens
agugcgugaauuuuccauauu

SEQ ID NO: 104 - RNA, 17 nt, Homo sapiens
uuuuccauaacagacuu

SEQ ID NO: 105 - RNA, 17 nt, Homo sapiens
cagaccuuguucccuuu

SEQ ID NO: 106 - RNA, 21 nt, Homo sapiens
ucacauuaagguggaagacuu

SEQ ID NO: 107 - RNA, 21 nt, Homo sapiens
gguggaagaccucuuuugauu

SEQ ID NO: 108 - RNA, 21 nt, Homo sapiens
gaccucuuuugaagauggauu

SEQ ID NO: 109 - RNA, 17 nt, Homo sapiens
gauggaaguggggaauu

SEQ ID NO: 110 - RNA, 17 nt, Homo sapiens
guggggaaugugaaguu

SEQ ID NO: 111 - RNA, 21 nt, Homo sapiens
guuuucuguaagaaccaucuu

SEQ ID NO: 112 - RNA, 21 nt, Homo sapiens
gaaccauccugccccauucuu

SEQ ID NO: 113 - RNA, 21 nt, Homo sapiens
ccauccugccccauucucauu

SEQ ID NO: 114 - RNA, 21 nt, Homo sapiens
aagucauuacuuuccacaauu

SEQ ID NO: 115 - RNA, 19 nt, Homo sapiens
gucauuacuuuccacaauu

SEQ ID NO: 116 - RNA, 17 nt, Homo sapiens
gaaggaaccauuugauu

SEQ ID NO: 117 - RNA, 21 nt, Homo sapiens
ggaaccauuugaacuagaauu

SEQ ID NO: 118 - RNA, 21 nt, Homo sapiens
ccauuugaacuagaagcauuu

SEQ ID NO: 119 - RNA, 21 nt, Homo sapiens
cuagaagcauuuuauacuauu

SEQ ID NO: 120 - RNA, 21 nt, Homo sapiens
gcauuuuauacuaauuuacuu

SEQ ID NO: 121 - RNA, 21 nt, Homo sapiens
uuuacaugaagugccuuauuu

SEQ ID NO: 122 - RNA, 17 nt, Homo sapiens
gugccuuauccugauuu

SEQ ID NO: 123 - RNA, 21 nt, Homo sapiens
gaauugggagcuucacuauuu

SEQ ID NO: 124 - RNA, 21 nt, Homo sapiens
uugggagcuucacuauucauu

SEQ ID NO: 125 - RNA, 21 nt, Homo sapiens
uguuuuuccacagucugauuu

SEQ ID NO: 126 - RNA, 21 nt, Homo sapiens
agugaagguuaaaguucguuu

SEQ ID NO: 127 - RNA, 21 nt, Homo sapiens
gguuaaaguucguguuaacuu

SEQ ID NO: 128 - RNA, 21 nt, Homo sapiens
aguucguguuaacauccauuu

SEQ ID NO: 129 - RNA, 21 nt, Homo sapiens
cauccauggaaucuucaguuu

SEQ ID NO: 130 - RNA, 21 nt, Homo sapiens
ucuucaguguggcuagcgcuu

SEQ ID NO: 131 - RNA, 21 nt, Homo sapiens
uugagaagcaaaauuuggauu

SEQ ID NO: 132 - RNA, 21 nt, Homo sapiens
gcaaaauuuggaaggcgauuu

SEQ ID NO: 133 - RNA, 21 nt, Homo sapiens
aauuuggaaggcgaucacauu

SEQ ID NO: 134 - RNA, 21 nt, Homo sapiens
uuuggaaggcgaucacaguuu

SEQ ID NO: 135 - RNA, 21 nt, Homo sapiens
ggcgaucacagugaugcucuu

SEQ ID NO: 136 - RNA, 17 nt, Homo sapiens
uggagacagaaacuuuu

SEQ ID NO: 137 - RNA, 17 nt, Homo sapiens
acuucauuuaaaaauuu

SEQ ID NO: 138 - RNA, 21 nt, Homo sapiens
aaaugaaaacaaagauaauuu

SEQ ID NO: 139 - RNA, 21 nt, Homo sapiens
augaaaacaaagauaauauuu

SEQ ID NO: 140 - RNA, 21 nt, Homo sapiens
aacaaagauaauauggauauu

SEQ ID NO: 141 - RNA, 21 nt, Homo sapiens
caaagauaauauggauaaauu

SEQ ID NO: 142 - RNA, 21 nt, Homo sapiens
agauaauauggauaaaauguu

SEQ ID NO: 143 - RNA, 21 nt, Homo sapiens
uauggauaaaaugcagguuuu

SEQ ID NO: 144 - RNA, 21 nt, Homo sapiens
aaugcagguugaucaagaauu

SEQ ID NO: 145 - RNA, 21 nt, Homo sapiens
ugcagguugaucaagaagauu

SEQ ID NO: 146 - RNA, 17 nt, Homo sapiens
gaagaagggcaucaauu

SEQ ID NO: 147 - RNA, 17 nt, Homo sapiens
gaagggcaucaaaaauu

SEQ ID NO: 148 - RNA, 17 nt, Homo sapiens
gggcaucaaaaauguuu

SEQ ID NO: 149 - RNA, 21 nt, Homo sapiens
aaaugucaugcugaacacauu

SEQ ID NO: 150 - RNA, 21 nt, Homo sapiens
augucaugcugaacacacuuu

SEQ ID NO: 151 - RNA, 21 nt, Homo sapiens
cacacuccagaagaggaaauu

SEQ ID NO: 152 - RNA, 21 nt, Homo sapiens
gaggaaauugaucauacaguu

SEQ ID NO: 153 - RNA, 21 nt, Homo sapiens
auugaucauacaggagccauu

SEQ ID NO: 154 - RNA, 17 nt, Homo sapiens
aacaaagucagcuguuu

SEQ ID NO: 155 - RNA, 21 nt, Homo sapiens
caaagucagcugucucagauu

SEQ ID NO: 156 - RNA, 21 nt, Homo sapiens
agucagcugucucagacaauu

SEQ ID NO: 157 - RNA, 21 nt, Homo sapiens
acaagaccgauuaaaucaguu

SEQ ID NO: 158 - RNA, 21 nt, Homo sapiens
gaccgauuaaaucagacacuu

SEQ ID NO: 159 - RNA, 21 nt, Homo sapiens
aucagacacuuaaaaaagguu

SEQ ID NO: 160 - RNA, 17 nt, Homo sapiens
aaaaggaaaagucaauu

SEQ ID NO: 161 - RNA, 17 nt, Homo sapiens
aaggaaaagucaaaauu

SEQ ID NO: 162 - RNA, 17 nt, Homo sapiens
ggaaaagucaaaaguuu

SEQ ID NO: 163 - RNA, 17 nt, Homo sapiens
aagucaaaaguauuguu

SEQ ID NO: 164 - RNA, 21 nt, Homo sapiens
aaguauugaucuaccgaucuu

SEQ ID NO: 165 - RNA, 21 nt, Homo sapiens
guauugaucuaccgauccauu

SEQ ID NO: 166 - RNA, 17 nt, Homo sapiens
cuaggccaagaucuuuu

SEQ ID NO: 167 - RNA, 17 nt, Homo sapiens
gaucuucucaacagcuu

SEQ ID NO: 168 - RNA, 21 nt, Homo sapiens
cagcuacauugaaaaugaguu

SEQ ID NO: 169 - RNA, 21 nt, Homo sapiens
aaugaggggaagaugaucauu

SEQ ID NO: 170 - RNA, 21 nt, Homo sapiens
ugaggggaagaugaucauguu

SEQ ID NO: 171 - RNA, 21 nt, Homo sapiens
gaugaucaugcaagauaaguu

SEQ ID NO: 172 - RNA, 19 nt, Homo sapiens
gauaaguuagagaaagauu

SEQ ID NO: 173 - RNA, 17 nt, Homo sapiens
guuagagaaagaaaguu

SEQ ID NO: 174 - RNA, 17 nt, Homo sapiens
agaaagaaaugaugcuu

SEQ ID NO: 175 - RNA, 21 nt, Homo sapiens
gaaaugaugcuaagaaugcuu

SEQ ID NO: 176 - RNA, 21 nt, Homo sapiens
augaugcuaagaaugccguuu

SEQ ID NO: 177 - RNA, 21 nt, Homo sapiens
gaaugccguugaagaauauuu

SEQ ID NO: 178 - RNA, 21 nt, Homo sapiens
ugccguugaagaauauguauu

SEQ ID NO: 179 - RNA, 21 nt, Homo sapiens
gaauauguauaugauuuuauu

SEQ ID NO: 180 - RNA, 21 nt, Homo sapiens
uauguauaugauuuuagaguu

SEQ ID NO: 181 - RNA, 21 nt, Homo sapiens
aaauucaucacuccagaaguu

SEQ ID NO: 182 - RNA, 21 nt, Homo sapiens
auucaucacuccagaagacuu

SEQ ID NO: 183 - RNA, 21 nt, Homo sapiens
gacuugaguaaacugucuguu

SEQ ID NO: 184 - RNA, 20 nt, Homo sapiens
acugucugcaguauuggauu

SEQ ID NO: 185 - RNA, 21 nt, Homo sapiens
aauuggcuuuaugaagacguu

SEQ ID NO: 186 - RNA, 21 nt, Homo sapiens
uuggcuuuaugaagacggauu

SEQ ID NO: 187 - RNA, 21 nt, Homo sapiens
gacggagaggaccaaccuauu

SEQ ID NO: 188 - RNA, 21 nt, Homo sapiens
ccuaaacaaguuuaugugguu

SEQ ID NO: 189 - RNA, 17 nt, Homo sapiens
acaaguuuauguggauu

SEQ ID NO: 190 - RNA, 17 nt, Homo sapiens
guuuauguggauaaguu

SEQ ID NO: 191 - RNA, 21 nt, Homo sapiens
gcuucaagaacuaaagaaauu

SEQ ID NO: 192 - RNA, 21 nt, Homo sapiens
gaacuaaagaaauacggccuu

SEQ ID NO: 193 - RNA, 21 nt, Homo sapiens
cuaaagaaauacggccagcuu

SEQ ID NO: 194 - RNA, 21 nt, Homo sapiens
agaaauacggccagccuauuu

SEQ ID NO: 195 - RNA, 21 nt, Homo sapiens
auacggccagccuauucaauu

SEQ ID NO: 196 - RNA, 21 nt, Homo sapiens
augaaguacauggagcauguu

SEQ ID NO: 197 - RNA, 17 nt, Homo sapiens
guacauggagcaugauu

SEQ ID NO: 198 - RNA, 21 nt, Homo sapiens
aagccuuaaaugacuuggguu

SEQ ID NO: 199 - RNA, 21 nt, Homo sapiens
gccuuaaaugacuugggaauu

SEQ ID NO: 200 - RNA, 21 nt, Homo sapiens
augacuugggaaaaaagauuu

SEQ ID NO: 201 - RNA, 21 nt, Homo sapiens
aaaagauccaacuugucauuu

SEQ ID NO: 202 - RNA, 21 nt, Homo sapiens
aagauccaacuugucaugauu

SEQ ID NO: 203 - RNA, 21 nt, Homo sapiens
gauccaacuugucaugaaauu

SEQ ID NO: 204 - RNA, 19 nt, Homo sapiens
cuugucaugaaagugauuu

SEQ ID NO: 205 - RNA, 17 nt, Homo sapiens
agugauagaagcuuauu

SEQ ID NO: 206 - RNA, 21 nt, Homo sapiens
gcuuauagaaacaaggauguu

SEQ ID NO: 207 - RNA, 21 nt, Homo sapiens
acaaggaugaaagauaugauu

SEQ ID NO: 208 - RNA, 21 nt, Homo sapiens
ggaugaaagauaugaucauuu

SEQ ID NO: 209 - RNA, 21 nt, Homo sapiens
agauaugaucaucuggaucuu

SEQ ID NO: 210 - RNA, 17 nt, Homo sapiens
auggaaaagguugaauu

SEQ ID NO: 211 - RNA, 17 nt, Homo sapiens
aagguugaaaaauguuu

SEQ ID NO: 212 - RNA, 17 nt, Homo sapiens
gguugaaaaauguauuu

SEQ ID NO: 213 - RNA, 21 nt, Homo sapiens
aaauguaucagugaugccauu

SEQ ID NO: 214 - RNA, 21 nt, Homo sapiens
auguaucagugaugccauguu

SEQ ID NO: 215 - RNA, 21 nt, Homo sapiens
uaguaagaugaaugcacaguu

SEQ ID NO: 216 - RNA, 17 nt, Homo sapiens
gaugaaugcacagaauu

SEQ ID NO: 217 - RNA, 17 nt, Homo sapiens
ugcacagaacaaacuuu

SEQ ID NO: 218 - RNA, 17 nt, Homo sapiens
caaacuaagucucacuu

SEQ ID NO: 219 - RNA, 21 nt, Homo sapiens
acuaagucucacucaagauuu

SEQ ID NO: 220 - RNA, 21 nt, Homo sapiens
gucucacucaagauccuguuu

SEQ ID NO: 221 - RNA, 21 nt, Homo sapiens
gauccugugguaaaaguuuuu

SEQ ID NO: 222 - RNA, 21 nt, Homo sapiens
aaguuucagaaauaguagcuu

SEQ ID NO: 223 - RNA, 21 nt, Homo sapiens
guuucagaaauaguagcaauu

SEQ ID NO: 224 - RNA, 19 nt, Homo sapiens
auaguagcaaagucaaauu

SEQ ID NO: 225 - RNA, 17 nt, Homo sapiens
agucaaaggaacugguu

SEQ ID NO: 226 - RNA, 17 nt, Homo sapiens
aggaacuggauaauuuu

SEQ ID NO: 227 - RNA, 21 nt, Homo sapiens
cuggauaauuucuguaaccuu

SEQ ID NO: 228 - RNA, 21 nt, Homo sapiens
uuucuguaaccccaucauuuu

SEQ ID NO: 229 - RNA, 21 nt, Homo sapiens
ccccaucauuuacaagcccuu

SEQ ID NO: 230 - RNA, 21 nt, Homo sapiens
gcccaaaccaaaagcagaauu

SEQ ID NO: 231 - RNA, 20 nt, Homo sapiens
accaaaagcagaaguuccuu

SEQ ID NO: 232 - RNA, 17 nt, Homo sapiens
aagcagaaguuccuguu

SEQ ID NO: 233 - RNA, 17 nt, Homo sapiens
gcagaaguuccugaauu

SEQ ID NO: 234 - RNA, 17 nt, Homo sapiens
guuccugaagacaaauu

SEQ ID NO: 235 - RNA, 21 nt, Homo sapiens
gacaaaccaaaagcuaauauu

SEQ ID NO: 236 - RNA, 21 nt, Homo sapiens
accaaaagcuaauagugaauu

SEQ ID NO: 237 - RNA, 21 nt, Homo sapiens
aagcuaauagugaacacaauu

SEQ ID NO: 238 - RNA, 21 nt, Homo sapiens
gcuaauagugaacacaauguu

SEQ ID NO: 239 - RNA, 21 nt, Homo sapiens
uagugaacacaauggcccauu

SEQ ID NO: 240 - RNA, 21 nt, Homo sapiens
cacaauggcccaauggauguu

SEQ ID NO: 241 - RNA, 21 nt, Homo sapiens
uggcccaauggauggacaguu

SEQ ID NO: 242 - RNA, 21 nt, Homo sapiens
uggauggacagaguggaacuu

SEQ ID NO: 243 - RNA, 17 nt, Homo sapiens
cugaaacuaaaucaguu

SEQ ID NO: 244 - RNA, 21 nt, Homo sapiens
acuaaaucagauucaacaauu

SEQ ID NO: 245 - RNA, 21 nt, Homo sapiens
aucagauucaacaaaagacuu

SEQ ID NO: 246 - RNA, 21 nt, Homo sapiens
caaaagacagcucacagcauu

SEQ ID NO: 247 - RNA, 21 nt, Homo sapiens
aagacagcucacagcauacuu

SEQ ID NO: 248 - RNA, 21 nt, Homo sapiens
gacagcucacagcauacuauu

SEQ ID NO: 249 - RNA, 17 nt, Homo sapiens
auccucuggagagauuu

SEQ ID NO: 250 - RNA, 21 nt, Homo sapiens
guggacuaagucuuaauuuuu

SEQ ID NO: 251 - RNA, 21 nt, Homo sapiens
gucuuaauuuuaccuucacuu

SEQ ID NO: 252 - RNA, 21 nt, Homo sapiens
uuuuaccuucacauuaauuuu

SEQ ID NO: 253 - RNA, 21 nt, Homo sapiens
uucaaaccgugcaaguaacuu

SEQ ID NO: 254 - RNA, 20 nt, Homo sapiens
accgugcaaguaaccacguu

SEQ ID NO: 255 - RNA, 17 nt, Homo sapiens
guaaccacgggguccuu

SEQ ID NO: 256 - RNA, 17 nt, Homo sapiens
ccacgggguccaucuuu

SEQ ID NO: 257 - RNA, 21 nt, Homo sapiens
cagacgcucaguuguucuuuu

SEQ ID NO: 258 - RNA, 17 nt, Homo sapiens
ccacuuuugucauuuuu

SEQ ID NO: 259 - RNA, 21 nt, Homo sapiens
aaguguuuuauauugaguguu

SEQ ID NO: 260 - RNA, 21 nt, Homo sapiens
guguuuuauauugagugcauu

SEQ ID NO: 261 - RNA, 21 nt, Homo sapiens
uuagauuuacaagacaaucuu

SEQ ID NO: 262 - RNA, 18 nt, Homo sapiens
gacaaucuaagcuuucuu

SEQ ID NO: 263 - RNA, 17 nt, Homo sapiens
ucuaagcuuuccggauu

SEQ ID NO: 264 - RNA, 17 nt, Homo sapiens
gcuuuccggauaauuuu

SEQ ID NO: 265 - RNA, 21 nt, Homo sapiens
uuuuauauaucaaacauacuu

SEQ ID NO: 266 - RNA, 21 nt, Homo sapiens
acauacaggauggauacauuu

SEQ ID NO: 267 - RNA, 17 nt, Homo sapiens
cagucuaccuuauuuuu

SEQ ID NO: 268 - RNA, 21 nt, Homo sapiens
agcuucuacugggauaaacuu

SEQ ID NO: 269 - RNA, 21 nt, Homo sapiens
accucaauuccuuuauucauu

SEQ ID NO: 270 - RNA, 21 nt, Homo sapiens
uuccuuuauucaggaaagguu

SEQ ID NO: 271 - RNA, 17 nt, Homo sapiens
aggauacuuuauugcuu

SEQ ID NO: 272 - RNA, 21 nt, Homo sapiens
gcauagauuuaauugcaucuu

SEQ ID NO: 273 - RNA, 21 nt, Homo sapiens
uugcaucuuuauuuugaaauu

SEQ ID NO: 274 - RNA, 17 nt, Homo sapiens
aaacaaaugaaaauuuu

SEQ ID NO: 275 - RNA, 17 nt, Homo sapiens
acaaaugaaaauugauu

SEQ ID NO: 276 - RNA, 17 nt, Homo sapiens
augaaaauugauggguu

SEQ ID NO: 277 - RNA, 17 nt, Homo sapiens
aauugaugggguuuauu

SEQ ID NO: 278 - RNA, 17 nt, Homo sapiens
uugaugggguuuaaauu

SEQ ID NO: 279 - RNA, 21 nt, Homo sapiens
agcuacagaggcacugaccuu

SEQ ID NO: 280 - RNA, 17 nt, Homo sapiens
uuuaaauauuaaaacuu

SEQ ID NO: 281 - RNA, 21 nt, Homo sapiens
auauuaaaacaaauaagaguu

SEQ ID NO: 282 - RNA, 21 nt, Homo sapiens
aacaaauaagagcuuucccuu

SEQ ID NO: 283 - RNA, 21 nt, Homo sapiens
caaauaagagcuuucccaauu

SEQ ID NO: 284 - RNA, 21 nt, Homo sapiens
auaagagcuuucccaaaaauu

SEQ ID NO: 285 - RNA, 21 nt, Homo sapiens
gagcuuucccaaaaaaaaauu

SEQ ID NO: 286 - DNA, 20 bp, Homo sapiens
gtccctgaagatgtcaatgc

SEQ ID NO: 287 - DNA, 20 bp, Homo sapiens
atgtcaatgccacatgtcca

SEQ ID NO: 288 - protein, 30 aa, Artificial Sequence
  Triple Hemagglutinin motif
YPYDVPDYAGYPYDVPDYAGSYPYDVPDYA

SEQ ID NO: 289 - DNA, 65 bp, Artificial Sequence
  PCR Primer for ApoB29
attgccagcattgctaaagaagaaggggtatcactactcaagaggaaaatgtcagcctggtcgtc

SEQ ID NO: 290 - DNA, 66 bp, Artificial Sequence
  Reverse PCR Primer ApoB29
gggatagcccgcatactcaggaacatcgtatgggtaatcgatactgtaggaggcggaccagttgct